(19) United States
(12) Patent Application Publication
(10) Pub. No.: US 2015/0315279 A1
(43) Pub. Date: Nov. 5, 2015

(54) ANTIBODIES THAT BIND MEMBRANE-BOUND IL1RAP

(71) Applicant: CELLERANT THERAPEUTICS, INC., San Carlos, CA (US)

(72) Inventors: Ying-Ping JIANG, Lafayette, CA (US); J Yun TSO, Menlo Park, CA (US); Holger KARSUNKY, Redwood City, CA (US)

(21) Appl. No.: 14/652,925

(22) PCT Filed: Dec. 20, 2013
(86) PCT No.: PCT/US2013/077323

§ 371 Date: Jun. 17, 2015

Related U.S. Application Data
(60) Provisional application No. 61/745,337, filed on Dec. 21, 2012.
(60) Provisional application No. 61/779,249, filed on Mar. 13, 2013.

(51) Int. Cl. C07K 16/28

(52) U.S. Cl. CPC C07K 16/2866; C07K 2317/565; C07K 2317/34; C07K 2317/24; C07K 2317/92

(57) ABSTRACT

Methods and compositions for binding to the membrane-bound form of IL1 RAP. In some embodiments, the invention provides an isolated antibody that binds the membrane bound fom1 of human IL1 RAP. In some cases, the isolated antibody that binds the membrane-bound form of human IL1 RAP does not bind, or does not substantially bind, the soluble form of human IL1 RAP. For example, in some cases, the isolated antibody that binds the membrane-bound form of human IL1RAP does not bind, or does not substantially bind the soluble form of human IL1RAP that is found in normal human serum. In some cases, the soluble form of human IL1 RAP comprises the sequence GNRCGQ.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file “Sequence Listing for 92950-896200 (00332PC)”, created Dec. 20, 2013, 70,397 bytes, machine format IBM-PC, MS-Windows operating system, is hereby incorporated by reference in its entirety for all purposes.

### BACKGROUND OF THE INVENTION

Interleukin-1 (IL1) is a central regulator of both acute and chronic inflammatory responses mediated by the immune system. Interleukin-1 receptor accessory protein (IL1RAP) is a co-receptor for IL1 that functions in the IL1 receptor signal transduction complex and in some contexts is necessary to link cell membrane events to downstream signalling pathways.

IL1RAP is also expressed on cancer cells and cancer stem cells (CSCs), which are cells that can give rise to additional cancer cells.

One of the major limitations of chemotherapy is the general inability of anticancer drugs to discriminate between normal and cancer cells. Almost all members of the major categories of antineoplastic agents have considerable toxicity for normal cells.

Compositions that specifically target cancer cells can avoid this problem. However, existing cancer treatments do not target CSCs. For this reason, existing chemotherapeutic strategies, even when specifically delivered to cancer cells, do not effectively eliminate the cancer. Risk of recurrence remains because the surviving CSCs can give rise to new cancer cells.

Provided herein are anti-IL1RAP antibodies that bind to IL1RAP expressing cells. These antibodies provide novel diagnostic and therapeutic strategies for targeting IL1RAP-associated disorders.

### BRIEF SUMMARY OF THE INVENTION

In some embodiments, the invention provides an isolated antibody that binds the membrane bound form of human IL1RAP. In some cases, the isolated antibody that binds the membrane-bound form of human IL1RAP does not bind, or does not substantially bind, the soluble form of human IL1RAP. For example, in some cases, the the isolated antibody that binds the membrane-bound form of human IL1RAP does not bind, or does not substantially bind, the soluble form of human IL1RAP that is found in normal human serum. In some cases, the soluble form of human IL1RAP comprises the sequence GNRCGQ (SEQ ID NO: 1).

In some aspects, the antibody binds to a membrane-bound form of IL1RAP with at least 2, 5, 10, 25, 50, 100, or 1000-fold greater affinity than a soluble form of IL1RAP.

In some aspects, the antibody is a humanized antibody.

In some aspects, the antibody binds to membrane-bound IL1RAP with an Kd of less than about 1, 2, 3, 4, 5, 6, 7, 10, 15, 20, 25, 30, 40, 50 or 250 nM.

Also provided is an isolated antibody that binds an extracellular membrane-anchor-proximal region of human IL1RAP.

Also provided is an isolated antibody comprising the CDRs of an antibody produced by immunizing an animal against an antigen comprising an extracellular membrane-anchor-proximal region of human IL1RAP.

In some aspects, the antibodies bind an amino acid primary sequence that is within 10 to 15, 15-20, 25-30, or 35-40 amino acids of amino acid 361 of human ILR1RAP, wherein human IL1RAP comprises the amino acid sequence set forth in Genebank accession Q9NPH3 (SEQ ID NO: 2).

In some aspects, the extracellular membrane-anchor-proximal region comprises an amino acid primary sequence VPAPRYTVELAC (SEQ ID NO: 3).

In some aspects the extracellular membrane-anchor proximal region comprises an amino acid primary sequence

(SEQ ID NO: 4)

HARSAKGEVAKAAKVKQKVPAPRYTVELACGFGATC

In some aspects, the antibody binds the amino acid primary sequence

(SEQ ID NO: 3)

VPAPRYTVELAC.

In some aspects, the antibody binds the amino acid primary sequence

(SEQ ID NO: 4)

HARSAKGEVAKAAKVKQKVPAPRYTVELACGFGATC.

In some aspects, the isolated antibody that binds an extracellular membrane-anchor-proximal region of human IL1RAP binds to a membrane-bound form of IL1RAP but does not substantially bind to a soluble form of IL1RAP. For example, in some cases, the antibody does not substantially bind the soluble form of human IL1RAP that is found in normal human serum. As another example, in some cases, the antibody does not substantially bind the soluble form of human IL1RAP comprising the sequence

(SEQ ID NO: 1)

GNRCGQ.

In other aspects, the antibody binds to a membrane-bound form of IL1RAP but does not bind to a soluble form of IL1RAP. For example, the soluble form of IL1RAP found in normal human serum. As another example, the soluble form of IL1RAP comprising the sequence GNRCGQ (SEQ ID NO: 1).

In some aspects, the antibody binds to a membrane-bound form of IL1RAP with at least 2, 5, 10, 25, 50, 100, or 1000-fold greater affinity than the antibody binds to a soluble form of IL1RAP.

In some aspects, the antibody is a humanized antibody.

In some aspects, the antibody binds to membrane-bound IL1RAP with an Kd of less than about 1, 2, 3, 4, 5, 6, 7, 10, 15, 20, 25, 30, 40, 50 or 250 nM.

In some aspects of any of the foregoing aspects embodiments or cases, the antibody inhibits cancer stem cell growth, metabolic activity, viability, or division.

In some aspects of any of the foregoing aspects embodiments or cases, the antibody comprises:

one, two, or three heavy chain complementarity determining regions of any one of antibodies 1F5, 12F1, 42E1, 4G9, or 4B6; or

one, two, or three light chain complementarity determining regions of any one of antibodies 1F5, 12F1, 42E1, 4G9, or 4B6.

In some aspects, the antibody light chain variable region comprises an amino acid sequence that is at least 85%, 90%, 95%, or 100% identical to SEQ ID NO: 5, and/or the antibody heavy chain variable region comprises an amino acid sequence that is at least 85%, 90%, 95%, or 100% identical to SEQ ID NO: 6.

In some aspects, the antibody comprises the heavy chain and light chain complementarity determining regions of antibody clone 1F5.

In some aspects, the antibody light chain variable region sequence is SEQ ID NO: 5 and the heavy chain variable region sequence is SEQ ID NO: 6.

In some aspects, one, two, three, four, five, or six antibody complementarity determining regions of the antibody are selected from the group consisting of SEQ ID NO: 7, SEQ ID NO: 8, SEQ ID NO: 9, SEQ ID NO: 10, SEQ ID NO: 11, and SEQ ID NO: 12.

In some aspects, the antibody comprises a heavy chain variable region comprising SEQ ID NO: 7, SEQ ID NO: 8, and SEQ ID NO: 9, and/or a light chain variable region comprising SEQ ID NO: 10, SEQ ID NO: 11, and SEQ ID NO: 12.

In some aspects, the antibody comprises a complementarity determining region heavy chain 1 (CDRH1) at least 85%, 90%, 95%, or 100% identical to SEQ ID NO: 7; a complementarity determining region heavy chain 2 (CDRH2) at least 85%, 90%, 95%, or 100% identical to SEQ ID NO: 8; a complementarity determining region heavy chain 3 (CDRH3) at least 85%, 90%, 95%, or 100% identical to SEQ ID NO: 9; a complementarity determining region light chain 1 (CDRL1) at least 85%, 90%, 95%, or 100% identical to SEQ ID NO: 10; a complementarity determining region light chain 2 (CDRL2) at least 85%, 90%, 95%, or 100% identical to SEQ ID NO: 11; and a complementarity determining region light chain 3 (CDRL3) at least 85%, 90%, 95%, or 100% identical to SEQ ID NO: 12.

In some cases, the antibody comprises a CDRH1 differing by 1, 2, 3, or 4 amino acid substitutions from SEQ ID NO: 7; a CDRH2 differing by 1, 2, 3, or 4 amino acid substitutions from SEQ ID NO: 8; a CDRH3 differing by 1, 2, 3, or 4 amino acid substitutions from SEQ ID NO: 9; a CDRL1 differing by 1, 2, 3, or 4 amino acid substitutions from SEQ ID NO: 10; a CDRL2 differing by 1, 2, 3, or 4 amino acid substitutions from SEQ ID NO: 11; and a CDRL3 differing by 1, 2, 3, or 4 amino acid substitutions from SEQ ID NO: 12. In some cases, the substitutions are conservative substitutions.

In some aspects, the antibody light chain variable region comprises an amino acid sequence that is at least 85%, 90%, 95%, or 100% identical to the antibody light chain variable region of antibody 12F1, and/or the heavy chain variable region comprises an amino acid sequence that is at least 85%, 90%, 95%, or 100% identical to the antibody heavy chain variable region of antibody 12F1.

In some aspects, the antibody comprises heavy chain and light chain complementarity determining regions of antibody clone 12F1.

In some aspects, the antibody comprises one, two, three, four, five, or six antibody complementarity determining regions of antibody clone 12F1.

In some aspects, the antibody comprises a heavy chain variable region comprising CDRH1, CDRH2, and CDRH3 of antibody clone 12F1, and/or a light chain variable region comprising CDRL1, CDRL2, and CDRL3 of antibody clone 12 F1.

In some aspects, the antibody comprises a CDRH1 at least 85%, 90%, 95%, or 100% identical to a CDRH1 of antibody clone 12F1; a CDRH2 at least 85%, 90%, 95%, or 100% identical to a CDRH2 of antibody clone 12F1; a CDRH3 at least 85%, 90%, 95%, or 100% identical to a CDRH3 of antibody clone 12F1; a CDRL1 at least 85%, 90%, 95%, or 100% identical to a CDRL1 of antibody clone 12F1; a CDRL2 at least 85%, 90%, 95%, or 100% identical to a CDRL2 of antibody clone 12F1; and a CDRL3 at least 85%, 90%, 95%, or 100% identical to a CDRL3 of antibody clone 12F1.

In some cases, the antibody comprises a CDRH1 differing by 1, 2, 3, or 4 amino acid substitutions from a CDRH1 of antibody clone 12F1; a CDRH2 differing by 1, 2, 3, or 4 amino acid substitutions from a CDRH2 of antibody clone 12F1; a CDRH3 differing by 1, 2, 3, or 4 amino acid substitutions from a CDRH3 of antibody clone 12F1; a CDRL1 differing by 1, 2, 3, or 4 amino acid substitutions from a CDRL1 of antibody clone 12F1; a CDRL2 differing by 1, 2, 3, or 4 amino acid substitutions from a CDRL2 of antibody clone 12F1; and a CDRL3 differing by 1, 2, 3, or 4 amino acid substitutions from a CDRL3 of antibody clone 12F1. In some cases, the substitutions are conservative substitutions.

In some aspects, the antibody light chain variable region comprises an amino acid sequence that is at least 85%, 90%, 95%, or 100% identical to the antibody light chain variable region of antibody 42E1, and/or the heavy chain variable region comprises an amino acid sequence that is at least 85%, 90%, 95%, or 100% identical to the antibody heavy chain variable region of antibody 42E1.

In some aspects, the antibody comprises heavy chain and light chain complementarity determining regions of antibody clone 42E1.

In some aspects, the antibody comprises one, two, three, four, five, or six antibody complementarity determining regions of antibody clone 42E1.

In some aspects, the antibody comprises a heavy chain variable region comprising CDRH1, CDRH2, and CDRH3 of antibody clone 42E1, and/or a light chain variable region comprising CDRL1, CDRL2, and CDRL3 of antibody clone 42E1.

In some aspects, the antibody comprises a CDRH1 at least 85%, 90%, 95%, or 100% identical to a CDRH1 of antibody clone 42E1; a CDRH2 at least 85%, 90%, 95%, or 100% identical to a CDRH2 of antibody clone 42E1; a CDRH3 at least 85%, 90%, 95%, or 100% identical to a CDRH3 of antibody clone 42E1; a CDRL1 at least 85%, 90%, 95%, or 100% identical to a CDRL1 of antibody clone 42E1; a CDRL2 at least 85%, 90%, 95%, or 100% identical to a CDRL2 of antibody clone 42E1; and a CDRL3 at least 85%, 90%, 95%, or 100% identical to a CDRL3 of antibody clone 42E1.

In some cases, the antibody comprises a CDRH1 differing by 1, 2, 3, or 4 amino acid substitutions from a CDRH1 of antibody clone 42E1; a CDRH2 differing by 1, 2, 3, or 4 amino acid substitutions from a CDRH2 of antibody clone 42E1; a CDRH3 differing by 1, 2, 3, or 4 amino acid substitutions from a CDRH3 of antibody clone 42E1; a CDRL1 differing by 1, 2, 3, or 4 amino acid substitutions from a CDRL1 of antibody clone 42E1; a CDRL2 differing by 1, 2, 3, or 4 amino acid substitutions from a CDRL2 of antibody clone 42E1; and a CDRL3 differing by 1, 2, 3, or 4 amino acid substitutions from a CDRL3 of antibody clone 42E1. In some cases, the substitutions are conservative substitutions.

In some aspects, the antibody light chain variable region comprises an amino acid sequence that is at least 85%, 90%, 95%, or 100% identical to SEQ ID NO: 13, and/or the antibody heavy chain variable region comprises an amino acid sequence that is at least 85%, 90%, 95%, or 100% identical to SEQ ID NO: 14.

In some aspects, the antibody comprises heavy chain and light chain complementarity determining regions of antibody clone 4G9.

In some aspects, the antibody light chain variable region sequence is SEQ ID NO: 13 and the heavy chain variable region sequence is SEQ ID NO: 14.

In some aspects, one, two, three, four, five, or six antibody complementarity determining regions of the antibody are selected from the group consisting of SEQ ID NO: 15, SEQ ID NO: 16, SEQ ID NO: 17, SEQ ID NO: 18, SEQ ID NO: 19, and SEQ ID NO: 20.

In some aspects, the antibody comprises a heavy chain variable region comprising SEQ ID NO: 15, SEQ ID NO: 16, and SEQ ID NO: 17, and/or a light chain variable region comprising SEQ ID NO: 18, SEQ ID NO: 19, and SEQ ID NO: 20.

In some aspects, the antibody comprises a CDRH1 at least 85%, 90%, 95%, or 100% identical to SEQ ID NO: 15; a CDRH2 at least 85%, 90%, 95%, or 100% identical to SEQ ID NO: 16; a CDRH3 at least 85%, 90%, 95%, or 100% identical to SEQ ID NO: 17; a CDRL1 at least 85%, 90%, 95%, or 100% identical to SEQ ID NO: 18; a CDRL2 at least 85%, 90%, 95%, or 100% identical to SEQ ID NO: 19; and a CDRL3 at least 85%, 90%, 95%, or 100% identical to SEQ ID NO: 20.

In some cases, the antibody comprises a CDRH1 differing by 1, 2, 3, or 4 amino acid substitutions from SEQ ID NO: 15; a CDRH2 differing by 1, 2, 3, or 4 amino acid substitutions from SEQ ID NO: 16; a CDRH3 differing by 1, 2, 3, or 4 amino acid substitutions from SEQ ID NO: 17; a CDRL1 differing by 1, 2, 3, or 4 amino acid substitutions from SEQ ID NO: 18; a CDRL2 differing by 1, 2, 3, or 4 amino acid substitutions from SEQ ID NO: 19; and a CDRL3 differing by 1, 2, 3, or 4 amino acid substitutions from SEQ ID NO: 20. In some cases, the substitutions are conservative substitutions.

In some aspects, the antibody light chain variable region comprises an amino acid sequence that is at least 85%, 90%, 95%, or 100% identical to SEQ ID NO: 21 or the antibody heavy chain variable region comprises an amino acid sequence that is at least 85%, 90%, 95%, or 100% identical to SEQ ID NO: 22.

In some aspects, the antibody comprises heavy chain and light chain complementarity determining regions of antibody clone 4B6.

In some aspects, the antibody light chain variable region sequence is SEQ ID NO: 21 and the heavy chain variable region sequence is SEQ ID NO: 22.

In some aspects, one, two, three, four, five, or six antibody complementarity determining regions of the antibody are selected from the group consisting of SEQ ID NO: 23, SEQ ID NO: 24, SEQ ID NO: 25, SEQ ID NO: 26, SEQ ID NO: 27, and SEQ ID NO: 28.

In some aspects, the antibody comprises a heavy chain variable region comprising SEQ ID NO: 23. SEQ ID NO: 24, and SEQ ID NO: 25, and/or a light chain variable region comprising SEQ ID NO: 26, SEQ ID NO: 27, and SEQ ID NO: 28.

In some aspects, the antibody comprises a heavy chain variable region comprising:
a CDRH1 sequence of SEQ ID NO: 23, a CDRH2 sequence of SEQ ID NO: 24, and a CDRH3 sequence of SEQ ID NO: 25; or
a CDRH11 sequence of SEQ ID NO: 51, a CDRH2 sequence of SEQ ID NO: 52, and a CDRH3 sequence of SEQ ID NO: 53, and/or
   the antibody comprises a light chain variable region comprising
a CDRL1 sequence of SEQ ID NO: 26, a CDRL2 sequence of SEQ ID NO: 27, and a CDRL3 sequence of SEQ ID NO: 28; or
a CDRL1 sequence of SEQ ID NO: 54, a CDRL2 sequence of SEQ ID NO: 55, and a CDRL3 sequence of SEQ ID NO: 56.

In some aspects, the antibody comprises a CDRH1 at least 85%, 90%, 95%, or 100% identical to SEQ ID NO: 23; a CDRH2 at least 85%, 90%, 95%, or 100% identical to SEQ ID NO: 24; a CDRH3 at least 85%, 90%, 95%, or 100% identical to SEQ ID NO: 25; a CDRL1 at least 85%, 90%, 95%, or 100% identical to SEQ ID NO: 26; a CDRL2 at least 85%, 90%, 95%, or 100% identical to SEQ ID NO: 27; and a CDRL3 at least 85%, 90%, 95%, or 100% identical to SEQ ID NO: 28.

In some cases, the antibody comprises a CDRH1 differing by 1, 2, 3, or 4 amino acid substitutions from SEQ ID NO: 23; a CDRH2 differing by 1, 2, 3, or 4 amino acid substitutions from SEQ ID NO: 24; a CDRH3 differing by 1, 2, 3, or 4 amino acid substitutions from SEQ ID NO: 25; a CDRL1 differing by 1, 2, 3, or 4 amino acid substitutions from SEQ ID NO: 26; a CDRL2 differing by 1, 2, 3, or 4 amino acid substitutions from SEQ ID NO: 27; and a CDRL3 differing by 1, 2, 3, or 4 amino acid substitutions from SEQ ID NO: 28. In some cases, the substitutions are conservative substitutions.

In some aspects, the antibody light chain variable region comprises an amino acid sequence that is at least 85%, 90%, 95%, or 100% identical to at least 90 consecutive amino acids of SEQ ID NO: 48, or the antibody heavy chain variable region comprises an amino acid sequence that is at least 85%, 90%, 95%, or 100% identical to at least 90 consecutive amino acids of SEQ ID NO: 50.

In some aspects, the antibody light chain variable region comprises an amino acid sequence that is at least 85%, 90%, 95%, or 100% identical to at least 90 consecutive amino acids of SEQ ID NO: 44, or the antibody heavy chain variable region comprises an amino acid sequence that is at least 85%, 90%, 95%, or 100% identical to at least 90 consecutive amino acids of SEQ ID NO: 46.

In some aspects, the antibody light chain variable region comprises an amino acid sequence that is at least 85%, 90%, 95%, or 100% identical to at least 90 consecutive amino acids of SEQ ID NO: 48, or the antibody heavy chain variable region comprises an amino acid sequence that is at least 85%, 90%, 95%, or 100% identical to at least 90 consecutive amino acids of SEQ ID NO: 46.

In some aspects, the antibody light chain variable region comprises an amino acid sequence that is at least 85%, 90%, 95%, or 100% identical to at least 90 consecutive amino acids of SEQ ID NO: 44, or the antibody heavy chain variable region comprises an amino acid sequence that is at least 85%, 90%, 95%, or 100% identical to at least 90 consecutive amino acids of SEQ ID NO: 50.

In some aspects, the light chain variable region sequence is 100% identical to at least 90 (e.g., at least about 90, 95, 100, 105, or 110) consecutive amino acids of SEQ ID NO: 48, and the heavy chain variable region sequence is 100% identical to at least 90 (e.g., at least about 90, 95, 100, 105, or 110) consecutive amino acids of SEQ ID NO: 50.

In some aspects, the light chain variable region sequence is 100% identical to at least 90 (e.g., at least about 90, 95, 100, 105, or 110) consecutive amino acids of SEQ ID NO: 44, and the heavy chain variable region sequence is 100% identical to at least 90 (e.g., at least about 90, 95, 100, 105, or 110) consecutive amino acids of SEQ ID NO: 46.

In some aspects, the light chain variable region sequence is 100% identical to at least 90 (e.g., at least about 90, 95, 100, 105, or 110) consecutive amino acids of SEQ ID NO: 48, and the heavy chain variable region sequence is 100% identical to at least 90 (e.g., at least about 90, 95, 100, 105, or 110) consecutive amino acids of SEQ ID NO: 46.

In some aspects, the light chain variable region sequence is 100% identical to at least 90 (e.g., at least about 90, 95, 100, 105, or 110) consecutive amino acids of SEQ ID NO: 44, and the heavy chain variable region sequence is 100% identical to at least 90 (e.g., at least about 90, 95, 100, 105, or 110) consecutive amino acids of SEQ ID NO: 50.

In some aspects, one, two, three, four, five, or six antibody complementarity determining regions of the antibody are selected from the group consisting of SEQ ID NO: 51, SEQ ID NO: 52, SEQ ID NO: 53, SEQ ID NO: 54, SEQ ID NO: 55, and SEQ ID NO: 56.

In some aspects, one, two, three, four, five, or six antibody complementarity determining regions of the antibody are selected from the group consisting of SEQ ID NO: 51, SEQ ID NO: 52, SEQ ID NO: 53, SEQ ID NO: 54, SEQ ID NO: 55, SEQ ID NO: 56, SEQ ID NO: 23, SEQ ID NO: 24, SEQ ID NO: 25, SEQ ID NO: 26, SEQ ID NO: 27, and SEQ ID NO: 28.

In some aspects, the antibody comprises a CDRH1 at least 85%, 90%, 95%, or 100% identical to SEQ ID NO: 51; a CDRH2 at least 85%, 90%, 95%, or 100% identical to SEQ ID NO: 52; a CDRH3 at least 85%, 90%, 95%, or 100% identical to SEQ ID NO: 53; a CDRL1 at least 85%, 90%, 95%, or 100% identical to SEQ ID NO: 54; a CDRL2 at least 85%, 90%, 95%, or 100% identical to SEQ ID NO: 55; and a CDRL3 at least 85%, 90%, 95%, or 100% identical to SEQ ID NO: 56.

In some aspects, the antibody comprises a CDRH1 at least 85%, 90%, 95%, or 100% identical to SEQ ID NO: 51; a CDRH2 at least 85%, 90%, 95%, or 100% identical to SEQ ID NO: 52; a CDRH3 at least 85%, 90%, 95%, or 100% identical to SEQ ID NO: 53; a CDRL1 at least 85%, 90%, 95%, or 100% identical to SEQ ID NO: 26; a CDRL2 at least 85%, 90%, 95%, or 100% identical to SEQ ID NO: 27; and a CDRL3 at least 85%, 90%, 95%, or 100% identical to SEQ ID NO: 28.

In some aspects, the antibody comprises a CDRH1 at least 85%, 90%, 95%, or 100% identical to SEQ ID NO: 23; a CDRH2 at least 85%, 90%, 95%, or 100% identical to SEQ ID NO: 24; a CDRH3 at least 85%, 90%, 95%, or 100% identical to SEQ ID NO: 25; a CDRL1 at least 85%, 90%, 95%, or 100% identical to SEQ ID NO: 54; a CDRL2 at least 85%, 90%, 95%, or 100% identical to SEQ ID NO: 55; and a CDRL3 at least 85%, 90%, 95%, or 100% identical to SEQ ID NO: 56.

In some cases, the antibody comprises a CDRH1 differing by 1, 2, 3, or 4 amino acid substitutions from SEQ ID NO: 51; a CDRH2 differing by 1, 2, 3, or 4 amino acid substitutions from SEQ ID NO: 52; a CDRH3 differing by 1, 2, 3, or 4 amino acid substitutions from SEQ ID NO: 53; a CDRL1 differing by 1, 2, 3, or 4 amino acid substitutions from SEQ ID NO: 54; a CDRL2 differing by 1, 2, 3, or 4 amino acid substitutions from SEQ ID NO: 55; and a CDRL3 differing by 1, 2, 3, or 4 amino acid substitutions from SEQ ID NO: 56. In some cases, the substitutions are conservative substitutions.

In some cases, the antibody comprises a CDRH1 differing by 1, 2, 3, or 4 amino acid substitutions from SEQ ID NO: 51; a CDRH2 differing by 1, 2, 3, or 4 amino acid substitutions from SEQ ID NO: 52; a CDRH3 differing by 1, 2, 3, or 4 amino acid substitutions from SEQ ID NO: 53; a CDRL1 differing by 1, 2, 3, or 4 amino acid substitutions from SEQ ID NO: 26; a CDRL2 differing by 1, 2, 3, or 4 amino acid substitutions from SEQ ID NO: 27; and a CDRL3 differing by 1, 2, 3, or 4 amino acid substitutions from SEQ ID NO: 28. In some cases, the substitutions are conservative substitutions.

In some cases, the antibody comprises a CDRH1 differing by 1, 2, 3, or 4 amino acid substitutions from SEQ ID NO: 23; a CDRH2 differing by 1, 2, 3, or 4 amino acid substitutions from SEQ ID NO: 24; a CDRH3 differing by 1, 2, 3, or 4 amino acid substitutions from SEQ ID NO: 25; a CDRL1 differing by 1, 2, 3, or 4 amino acid substitutions from SEQ ID NO: 54; a CDRL2 differing by 1, 2, 3, or 4 amino acid substitutions from SEQ ID NO: 55; and a CDRL3 differing by 1, 2, 3, or 4 amino acid substitutions from SEQ ID NO: 56. In some cases, the substitutions are conservative substitutions.

In some aspects, one, two, three, four, five, or six antibody complementarity determining regions of the antibody are selected from the group consisting of SEQ ID NO: 29, SEQ ID NO: 30, SEQ ID NO: 31, SEQ ID NO: 32, SEQ ID NO: 33, and SEQ ID NO: 12.

In some aspects, the antibody comprises a heavy chain variable region comprising SEQ ID NO: 29, SEQ ID NO: 30, and SEQ ID NO: 31, and/or a light chain variable region comprising SEQ ID NO: 32, SEQ ID NO: 33, and SEQ ID NO: 12.

In some aspects, the antibody comprises a CDRH1 at least 85%, 90%, 95%, or 100% identical to SEQ ID NO: 29; a CDRH2 at least 85%, 90%, 95%, or 100% identical to SEQ ID NO: 30; a CDRH3 at least 85%, 90%, 95%, or 100% identical to SEQ ID NO: 31; a CDRL1 at least 85%, 90%, 95%, or 100% identical to SEQ ID NO: 32; a CDRL2 at least 85%, 90%, 95%, or 100% identical to SEQ ID NO: 33; and a CDRL3 at least 85%, 90%, 95%, or 100% identical to SEQ ID NO: 12.

In some cases, the antibody comprises a CDRH1 differing by 1, 2, 3, or 4 amino acid substitutions from SEQ ID NO: 29; a CDRH2 differing by 1, 2, 3, or 4 amino acid substitutions from SEQ ID NO: 30; a CDRH3 differing by 1, 2, 3, or 4 amino acid substitutions from SEQ ID NO: 31; a CDRL1 differing by 1, 2, 3, or 4 amino acid substitutions from SEQ ID NO: 32; a CDRL2 differing by 1, 2, 3, or 4 amino acid substitutions from SEQ ID NO: 33; and a CDRL3 differing by 1, 2, 3, or 4 amino acid substitutions from SEQ ID NO: 12. In some cases, the substitutions are conservative substitutions.

In some aspects of any of the foregoing embodiments, aspects, or cases, the antibody comprises CDRs of an antibody that binds to a membrane-bound form of IL1RAP but does not bind, or does not substantially bind, to a soluble form of IL1RAP.

In some aspects of any of the foregoing embodiments, aspects, or cases, the antibody is an Fab, F(ab′)2, an Fab′, or a single chain Fv. In some cases, the antibody is conjugated to a detectable label selected from the group consisting of a fluorophore, a radionuclide, a phosphorescent agent, a chemiluminescent agent, an enzyme, and an MRI contrast agent. In some cases the antibody is conjugated to a chemotherapeutic or a cytotoxic agent selected from the group consisting of an alkylating agent, an antimetabolites, an antitumor antibiotic, hydroxyurea, a platinum-based chemotherapeutic agent, a taxane, bortezomib, lenalidomide, and thalidomide.

In some aspects of any of the foregoing embodiments, aspects, or cases, the antibody binds to CD45+ cells, CD45+/CD38+ cells, CD34+ cells, or CD34− cells.

In some aspects of any of the foregoing embodiments, aspects, or cases, the antibody binds to cancer cells, myelodysplastic syndrome (MDS) cells, acute myeloid leukemia (AML) or chronic myelogenous leukemia (CML) cells, anaplastic large-cell lymphoma (ALCL) cells, Hodgkins Lymphoma cells, or Non Hodgkins Lymphoma cells such as EOL-1 cells or Karpas 299 cells.

In some aspects of any of the foregoing embodiments, aspects, or cases, the antibody specifically binds the receptor form of IL1RAP and at least 80% of cells in a culture of EOL-1 cells.

In some aspects of any of the foregoing embodiments, aspects, or cases, the antibody binds AML, CML, ALCL, Hodgkins Lymphoma, Non Hodgkins Lymphoma, or MDS stem cells.

In some aspects of any of the foregoing embodiments, aspects, or cases, the invention provides a formulation comprising a pharmaceutically acceptable excipient, and an anti-receptor IL1RAP antibody. In some cases, the anti-receptor IL1RAP antibody binds the membrane bound form of human IL1RAP, but does not bind or does not substantially bind, the soluble form of human IL1RAP. In some cases, the soluble form of human IL1RAP is the soluble form that is present in normal human serum. In some cases, the soluble form of human IL1RAP comprises SEQ ID NO: 1 GNRCGQ.

In some embodiments, the invention provides a method of treating a subject suffering from a disease or condition comprising administering an effective amount of an anti-receptor IL1RAP antibody, or a formulation comprising a pharmaceutically acceptable excipient, and an anti-receptor IL1RAP antibody. In some cases, the anti-receptor IL1RAP antibody binds the membrane bound form of human IL1RAP, but does not bind or does not substantially bind, the soluble form of human IL1RAP. In some cases, the soluble form of human IL1RAP is the soluble form that is present in normal human serum. In some cases, the soluble form of human IL1RAP comprises GNRCGQ (SEQ ID NO: 1).

In some aspects, the invention provides methods and compositions for treating cancer. In some cases, the cancer is AML, CML, ALCL, Hodgkins Lymphoma, or Non Hodgkins Lymphoma.

In some aspects, the invention provides methods and compositions for treating joint, bone, or muscle disease; a hereditary systemic auto-inflammatory disease; a systemic inflammatory disease; or a common inflammatory disease.

### BRIEF DESCRIPTION OF THE DRAWINGS

FIG. 1 depicts receptor (i.e., membrane-bound) and secreted forms of IL1RAP protein. The peptide (AC-Pep-1), which is only in receptor IL1RAP, used for immunizing animals to generate receptor-binding antibodies is indicated.

FIG. 2 depicts results from primary screening for antibodies that bind IL1RAP expressing EOL-1 cells by fluorescence activated cell sorting (FACS). 14 antibodies were chosen for further analysis.

FIG. 3 depicts results from ELISA experiments to confirm binding to a membrane-anchor proximal region. 5 antibodies were chosen as AC-Pep-1 binding antibodies, confirmed by ELISA.

FIG. 4 depicts results from a specificity analysis of identified antibodies that bind the receptor form of IL1RAP. JVM-3 cells, which do not express IL1RAP, do not show any antibody interaction with the cells.

FIG. 5 depicts results from a specificity analysis of identified antibodies that bind the receptor form of IL1RAP. FACS of EOL-1 cells, which do express IL1RAP, show that the antibody binds the cells.

FIG. 6 antibody 1F5 binds the receptor form of IL1RAP, but does not bind the secreted form of IL1RAP that is present in normal human serum. Antibody 12G6 does recognize both the secreted form of IL1RAP from normal human serum and the receptor form of IL1RAP.

FIG. 7 depicts anti-receptor IL1RAP antibody 1F5 binding to an ELISA plate coated with the extracellular domain of the receptor form of IL1RAP.

FIG. 8 depicts anti-receptor IL1RAP antibody 1F5 binding to a well of an ELISA plate coated with peptide or a keyhole limpet hemocyanin-peptide conjugate (KLH-peptide). The antibody shows no detectable binding to keyhole limpet hemocyanin (KLH).

FIG. 9 depicts anti-receptor IL1RAP antibody 1F5 binding to CD45+/CD38+/CD34+ and CD45+/CD38+/CD34− Bone Marrow-derived Mast Cells (BMMC) and Peripheral Blood Mononuclear Cells (PBMC).

FIG. 10 depicts the nucleotide sequence of 1F5 light chain variable region eDNA (SEQ ID NO: 35) along with the deduced amino acid sequence (SEQ ID NO: 36). The signal peptide is in italics. The first amino acid residue of the mature protein is doubly underlined. CDRL1, 2 and 3, as defined by Kabat et al., Sequences of Proteins of Immunological Interest (National Institutes of Health, Bethesda, Md., 1987 and 1991), are singly underlined.

FIG. 11 depicts the nucleotide sequence of 1F5 heavy chain variable region cDNA (SEQ ID NO: 37) along with the deduced amino acid sequence (SEQ ID NO: 38). The signal peptide is in italics. The first amino acid residue of the mature protein is doubly underlined. CDRH1, 2 and 3, as defined by Kabat et al., Sequences of Proteins of Immunological Interest (National Institutes of Health, Bethesda, Md., 1987 and 1991), are singly underlined.

FIG. 12 depicts specific binding of monoclonal antibodies 4G9, 12F1, and 42E1 to the extracellular domain (ECD) of IL1RAP and the ACPep-1 conjugated to KLH, but not to the secreted form of IL1RAP or KLH by an ELISA assay.

FIG. 13 depicts the nucleotide sequence of 4G9 light chain variable region cDNA (SEQ ID NO: 39) along with the deduced amino acid sequence (SEQ ID NO: 40). The signal peptide is in italics. The first amino acid residue of the mature protein is doubly underlined. CDRL1, 2 and 3, as defined by Kabat et al., Sequences of Proteins of Immunological Interest (National Institutes of Health, Bethesda, Md., 1987 and 1991), are singly underlined.

FIG. 14 depicts the nucleotide sequence of 4G9 heavy chain variable region cDNA (SEQ ID NO: 41) along with the deduced amino acid sequence (SEQ ID NO: 42). The signal peptide is in italics. The first amino acid residue of the mature protein is doubly underlined. CDRH1, 2 and 3, as defined by Kabat et al., Sequences of Proteins of Immunological Interest (National Institutes of Health, Bethesda, Md., 1987 and 1991), are singly underlined.

FIG. 15 depicts binding of monoclonal antibody 4B6 to the extracellular domain (ECD) of IL1RAP, but not to the recombinant or the natural secreted form of IL1RAP by an ELISA assay.

FIG. 16 depicts specific binding of monoclonal antibody 4B6 to two IL1RAP expressing AML cell lines (EOL and HNT-34) by flow cytometry analysis.

FIG. 17 depicts the nucleotide sequence of 4B6 light chain variable region cDNA (SEQ ID NO: 43) along with the deduced amino acid sequence (SEQ ID NO: 44). The signal peptide is in italics. The first amino acid residue of the mature protein is doubly underlined. CDRL1, 2 and 3, as defined by Kabat et al., Sequences of Proteins of Immunological Interest (National Institutes of Health, Bethesda, Md., 1987 and 1991), are singly underlined.

FIG. 18 depicts the nucleotide sequence of 4B6 heavy chain variable region cDNA (SEQ ID NO: 45) along with the deduced amino acid sequence (SEQ ID NO: 46). The signal peptide is in italics. The first amino acid residue of the mature protein is doubly underlined. CDRH1, 2 and 3, as defined by Kabat et al., Sequences of Proteins of Immunological Interest (National Institutes of Health, Bethesda, Md., 1987 and 1991), are singly underlined.

FIG. 19 depicts an alternate embodiment for the nucleotide sequence of 4B6 light chain variable region cDNA (SEQ ID NO: 47) along with the deduced amino acid sequence (SEQ ID NO: 48). The signal peptide is in italics. The first amino acid residue of the mature protein is doubly underlined. CDRL1, 2 and 3, as defined by Kabat et al., Sequences of Proteins of Immunological Interest (National Institutes of Health, Bethesda, Md., 1987 and 1991), are singly underlined.

FIG. 20 depicts an alternate embodiment for the nucleotide sequence of 4B6 heavy chain variable region cDNA (SEQ ID NO: 49) along with the deduced amino acid sequence (SEQ ID NO: 50). The signal peptide is in italics. The first amino acid residue of the mature protein is doubly underlined. CDRH1, 2 and 3, as defined by Kabat et al., Sequences of Proteins of Immunological Interest (National Institutes of Health, Bethesda, Md., 1987 and 1991), are singly underlined.

### DETAILED DESCRIPTION OF THE INVENTION

### I. Introduction

Provided herein are antibodies specific for the cell membrane-bound or receptor form of IL1RAP. Receptor IL1RAP specific antibodies can be used, for example, to deliver a cytotoxic agent specifically to IL1RAP-expressing cells, for example, cancer cells such as Cancer Stem Cells (CSC), leukemias, lymphomas, and solid tumor cells.

Also provided are receptor IL1RAP-specific antibodies that inhibit cancer cell growth in the absence of a cytotoxic agent, inhibit signaling mediated by interleukins 18, and 33, inhibit signaling mediated by interleukin 1 family members 5, 6, 8, or 9 (i.e., IL1F5, IL1F6, IL1F8, or IL1F9), induce antibody dependent cell-mediated cytotoxicity, block IL-1 dependent signaling, or a combination thereof. In some embodiments, the present receptor IL1RAP-specific antibodies further provoke complement dependent cytotoxicity (CDC).

Also provided are receptor IL1RAP-specific antibodies that are effective for reducing cancer or cancer stem cell growth, metabolic activity, viability, or division. In addition to, or in combination with the therapeutic applications, the anti-receptor IL1RAP antibodies disclosed herein are useful for in vivo and in vitro diagnostic agents.

### II. Definitions

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Springs Harbor Press (Cold Springs Harbor, N.Y. 1989). Any methods, devices and materials similar or equivalent to those described herein can be used in the practice of this invention. The following definitions are provided to facilitate understanding of certain terms used frequently herein and are not meant to limit the scope of the present disclosure.

As used herein “IL1RAP” refers to human interleukin 1 receptor accessory protein. “Receptor IL1RAP,” “membrane-bound IL1RAP,” or any variation thereof refers to the form of the protein that is present on the surface of cells that express IL1RAP. As such, receptor or membrane-bound IL1RAP also refers to this form of IL1RAP when it has been purified from the membrane or is otherwise membrane-free.

An exemplary Receptor IL1RAP amino acid sequence is depicted below as SEQ ID NO: 2:

```
MTLLWCVVSL YFYGILQSDA SERCDDWGLD TMRQIQVFED
                                                            70 80
EPARIKCPLF EHFLKFNYST AHSAGLTLIW YWTRQDRDLE
                90 100 110 120
EPINFRLPEN RISKEKDVLW FRPTLLNDTG NYTCMLRNTT
              130 140 150 160
YCSKVAFPLE VVQKDSCFNS PMKLPVHKLY IEYGIQRITC
              170 180 190 200
PNVDGYFPSS VKPTITWYMG CYKIQNFNNV IPEGMNLSFL
              210 220 230 240
IALISNNGNY TCVVTYPENG RTFHLTRTLT VKVVGSPKNA
              250 260 270 280
VPPVIHSPND HVVYEKEPGE ELLIPCTVYF SFLMDSRNEV
              290 300 310 320
WWTIDGKKPD DITIDVTINE SISHSRTEDE TRTQILSIKK
              330 340 350 360
VTSEDLKRSY VCHARSAKGE VAKAAKVKQK VPAPRYTVEL
              370 380 390 400
ACGFGATVLL VVILIVVYHV YWLEMVLFYR AHFGTDETIL
              410 420 430 440
DGKEYDIYVS YARNAEEEEF VLLTLRGVLE NEFGYKLCIF
              450 460 470 480
DRDSLPGGIV TDETLSFIQK SRRLLVVLSP NYVLQGTQAL
              490 500 510 520
LELKAGLENM ASRGNINVIL VQYKAVKETK VKELKRAKTV
              530 540 550 560
LTVIKWKGEK SKYPQGRFWK QLQVAMPVKK SPRRSSSDEQ
              570
GLSYSSLKNV
```

In some cases, receptor IL1RAP includes an intact transmembrane (or membrane anchor) region, or a portion thereof. For example, receptor IL1RAP may contain the transmembrane spanning region denoted by amino acids 370-388, 369-388, 368-388, 367-388, 366-388, 365-388, 364-388, 363-388, 362-388, 361-388, 370-395, 369-395, 368-395, 368-394, 368-393, 368-392, 368-391, 368-390, or 368-389 of Genbank accession Q9NPH3. Transmembrane regions may include regions of the IL1RAP primary sequence that are 20 or fewer (e.g., 19, 18, 17, 16, 15, 14, 13, 12, 11, 10, 9, 8, 7, 6, 5, 4, 3, 2, or 1) amino acids in the carboxy or amino direction distal from the above-mentioned residues. In some cases, anti-receptor IL1RAP antibodies provided herein bind to IL1RAP proteins comprising one or more of these transmembrane regions. In some cases, receptor IL1RAP is membrane-bound. For example, the receptor IL1RAP, or a portion thereof, can be anchored into the membrane of a cell.

In some cases, receptor IL1RAP is distinguished from soluble IL1RAP by the presence of the VPAPRYTVELAC (SEQ ID NO: 3) sequence, which sequence soluble IL1RAP can lack. In some cases, receptor IL1RAP is distinguished from soluble IL1RAP by presence of the HARSAKGEVAKAAKVKQKVPAPRYTVELACGFGATC (SEQ ID NO: 4) sequence, which sequence soluble IL1RAP can lack. In some cases, receptor IL1RAP is distinguished from soluble IL1RAP by the absence of the GNRCGQ (SEQ ID NO: 1) sequence, which sequence soluble IL1RAP can contain.

In some cases, anti-receptor IL1RAP antibodies provided herein bind to IL1RAP proteins comprising one or more of these transmembrane regions, but do not bind IL1RAP proteins that do not comprise one or more of these transmembrane regions. In some cases, anti-receptor IL1RAP antibodies provided herein bind IL1RAP proteins comprising one or more of the above transmembrane regions with a substantially lower dissociation constant (i.e., tighter binding) than IL1RAP proteins that do not comprise one or more of these transmembrane regions. For example, in some embodiments, anti-receptor IL1RAP antibodies provided herein can bind IL1RAP proteins comprising a transmembrane region with a 2, 3, 4, 5, 6, 8, 10, 15, 20, 25, 30, 40, 50, 100, or 1000-fold or greater affinity than Ill RAP proteins that do not include a transmembrane region.

As used herein, “membrane-anchor proximal region” refers to amino acids of the IL1RAP protein that are proximal to, but N-terminal of the transmembrane region. In some cases, receptor IL1RAP includes a membrane-anchor proximal region. One of skill in the art will know how to determine membrane-anchor proximal regions which can include, by way of example only, amino acids 300-370, 301-370, 302-370, 303-370, 304-370, 305-370, 306-370, 307-370, 308-370, 309-370, 310-370, 311-370, 312-370, 313-370, 314-370, 315-370, 316-370, 317-370, 318-370, 319-370, 320-370, 321-370, 322-370, 323-370, 324-370, 325-370, 326-370, 327-370, 328-370, 329-370, 330-370, 331-370, 332-370, 333-370, 334-370, 335-370, 336-370, 337-370, 338-370, 339-370, 340-370, 340-369, 340-368, 340-367, 340-366, 340-365, 340-364, 340-363, 340-362, 340-361, 340-360, 341-370, 341-369, 341-368, 341-367, 341-366, 341-365, 341-364, 341-363, 341-362, 341-361, 341-360, 342-370, 342-369, 342-368, 342-367, 342-366, 342-365, 342-364, 342-363, 342-362, 342-361, 342-360, 343-370, 343-369, 343-368, 343-367, 343-366, 343-365, 343-364, 343-363, 343-362, 343-361, 343-360, 344-370, 344-369, 344-368, 344-367, 344-366, 344-365, 344-364, 344-363, 344-362, 344-361, 344-360, 345-370, 345-369, 345-368, 345-367, 345-366, 345-365, 345-364, 345-363, 345-362, 345-361, 345-360, 346-370, 346-369, 346-368, 346-367, 346-366, 346-365, 346-364, 346-363, 346-362, 346-361, 346-360, 347-370, 347-369, 347-368, 347-367, 347-366, 347-365, 347-364, 347-363, 347-362, 347-361, or amino acids 347-360 of Genbank accession Q9NPH3. The membrane-anchor proximal region may include regions of the IL1RAP primary sequence that are 15, 14, 13, 12, 11, 10 or fewer amino acids in the carboxy or amino direction from the above-mentioned residues. In some cases, anti-receptor IL1RAP antibodies provided herein bind to IL1RAP proteins comprising one or more of these membrane-anchor proximal regions. In some cases, anti-receptor IL1RAP antibodies provided herein bind to IL1RAP proteins comprising one or more of these membrane-anchor proximal regions, but do not bind, or do not substantially bind IL1RAP proteins that do not comprise one or more of these membrane-anchor proximal regions.

In some embodiments, anti-receptor IL1RAP antibodies provided herein bind to amino acid residues within the membrane-anchor proximal regions described herein. For example, in some cases, anti-receptor IL1RAP antibodies provided herein can bind to residues within amino acids 300-368, 320-360, 330-350, or 347-362. In some cases, the anti-receptor IL1RAP antibodies provided herein bind to regions of the IL1RAP primary sequence that are 15, 14, 13, 12, 11, 10, 9, 8, 7, 6, 5, 4, 3, 2, or 1 amino acids in the carboxy or amino terminal direction from the above mentioned membrane-anchor proximal region.

As used herein “soluble IL1RAP” refers to naturally occurring IL1RAP proteins that are not membrane bound. Specific examples of soluble IL1RAP proteins include IL1RAP proteins that do not include one or more of the above-mentioned transmembrane regions, IL1RAP proteins that do not include one or more of the above-mentioned membrane-anchor proximal regions, IL1RAP proteins that are not present on the surface of IL1RAP expressing cells, IL1RAP proteins that have been proteolytically cleaved such that they are not membrane anchored, cytosolic IL1RAP, and IL1RAP proteins that are present in (e.g., secreted into) normal human serum. In one embodiment, soluble IL1RAP consists of the amino acid sequence SEQ ID NO: 34:

```
MTLLWCVVSL YFYGILQSDA SERCDDWGLD TMRQDQVFED
                                                            70 80
EPARIKCPLF EHFLKFNYST AHSAGLTLIW YWTRQDRDLE
                90 100 110 120
EPINFRLPEN RISKEKDVLW FRPTLLNDTG NYTCMLRNTT
              130 140 150 160
YCSKVAFPLE VVQKDSCFNS PMKLPVHKLY IEYGIQRITC
              170 180 190 200
PNVDGYFPSS VKPTITWYMG CYKIQNFNNV IPEGMNLSFL
              210 220 230 240
IALISNNGNY TCVVTYPENG RTFHLTRTLT VKVVGSPKNA
              250 260 270 280
VPPVIHSPND HVVYEKEPGE ELLIPCTVYF SFLMDSRNEV
              290 300 310 320
WWTIDGKKPD DITIDVTINE SISHSRTEDE TRTQILSIKK
              330 340 350 360
VTSEDLKRSY VCHARSAKGE VAKAAKVKQK GNRCGO
```

The terms “receptor IL1RAP-specific antibody,” “anti-receptor IL1RAP antibody,” “receptor IL1RAP antibody,” “anti-receptor IL1RAP,” “antibody that binds to a membrane-bound form of IL1RAP but does not bind to a soluble form” and the like are used synonymously herein to refer to an antibody that specifically binds to the receptor (i.e., membrane-bound) form of IL1RAP. In some cases, the receptor IL1RAP antibodies described herein specifically bind the AC-Pep 1 polypeptide. In some embodiments, the specific binding to receptor IL1RAP includes binding to receptor IL1RAP, but not binding to the soluble form of IL1RAP found in (e.g., secreted into) normal human serum. In some embodiments, the specific binding to receptor IL1RAP includes binding to receptor IL1RAP, but not binding to the soluble form of IL1RAP comprising the sequence GNRCGQ (SEQ ID NO: 1). In some embodiments, the specific binding to the receptor IL1RAP includes binding to the sequence VPAPRYTVELAC (SEQ ID NO: 3) and/or HARSAKGEVAKAAKVKQKVPAPRYTVELACGFGATC (SEQ ID NO: 4), which sequences can be found in receptor IL1RAP, but not soluble IL1RAP. In some cases, receptor IL1RAP-specific antibodies can bind to certain non-natural soluble forms of IL1RAP, such as a protein that contains the extracellular domain of receptor IL1RAP (ECD IL1RAP), including one or more membrane anchor proximal regions, but does not contain a transmembrane region, and/or does not contain GNRCGQ (SEQ ID NO: 1).

In some cases, the receptor IL1RAP antibodies specifically bind to IL1RAP present on the surface of cells such as certain cancer cells (e.g., cancer stem cells (CSCs) or hematopoietic tumor cells (HTCs)), but not to most mature peripheral blood cells. As discussed in more detail below, in some embodiments the present anti-receptor IL1RAP antibodies can bind IL1RAP expressing cells, inhibit their proliferation and/or mediate their destruction.

An “IL1RAP-associated disorder” (or IL1RAP related disorder, IL1RAP disorder, IL1RAP related condition or disease, etc.) refers to conditions and diseases correlated with elevated or reduced cell surface expression of IL1RAP as compared to IL1RAP expression in a standard control (e.g., a normal, non-disease, non-cancer cell). Elevated IL1RAP levels are associated with cancer cells, in particular, cancer stem cells.

The terms “engraft” or “engraftment” refers to the ability of a cell to survive, proliferate, and/or properly localize upon introduction into an individual or tissue. In the case of a cancer stem cell (CSC), the term can refer to the ability of the CSC to generate a tumor de novo or to spread to a different site. The term is commonly used to describe the ability of a population of cells to survive and function in a xenograft model (e.g., engraftment of human cells in a mouse). Engraftment of hematopoietic cells can be determined as described, e.g., in WO2006/047569. Engraftment of tumor cells can be determined as described, e.g., in Beckhove et al. (2003) Int. J. Cancer 105:444.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

A variety of methods of specific DNA and RNA measurements that use nucleic acid hybridization techniques are known to those of skill in the art (see, Sambrook, Id.). Some methods involve electrophoretic separation (e.g., Southern blot for detecting DNA, and Northern blot for detecting RNA), but measurement of DNA and RNA can also be carried out in the absence of electrophoretic separation (e.g., quantitative PCR, dot blot, or array).

The words “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following amino acids are typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see. e.g., Creighton, Proteins (1984)).

The terms “identical” or “percent identity,” in the context of two or more nucleic acids, or two or more polypeptides, refer to two or more sequences or subsequences that are the same or have a specified percentage of nucleotides, or amino acids, that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters, or by manual alignment and visual inspection. See e.g., the NCBI web site at ncbi.nlm.nih.gov/BLAST. Such sequences are then said to be “substantially identical.” This definition also refers to, or may be applied to, the compliment of a nucleotide test sequence. The definition also includes sequences that have deletions and/or additions, as well as those that have substitutions. As described below, the algorithms can account for gaps and the like. Typically, identity exists over a region comprising an antibody epitope, or a sequence that is at least about 25 amino acids or nucleotides in length, or over a region that is 50-100 amino acids or nucleotides in length, or over the entire length of the reference sequence.

An antibody can consist of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An “antibody” functions as a binding protein and is structurally defined as comprising an amino acid sequence from or derived from the framework region of an immunoglobulin encoding gene of an animal producing antibodies.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) or light chain variable region and heavy chain variable region and the like refer to these light and heavy chains and their variable regions respectively.

The term “antibody” as used herein includes antibody fragments that retain binding activity. For example, there are a number of well characterized antibody fragments. Thus, for example, pepsin digests an antibody C-terminal to the disulfide linkages in the hinge region to produce F(ab′)₂, a dimer of Fab which itself is a light chain joined to VH-CH1 by a disulfide bond. The F(ab′)₂ may be reduced under mild conditions to break the disulfide linkage in the hinge region thereby converting the F(ab′)₂ dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that fragments can be synthesized de novo either chemically or by utilizing recombinant DNA methodology. Thus, the term antibody, as used herein also includes antibody fragments either produced by the modification of whole antibodies or synthesized using recombinant DNA methodologies.

An “isotype” is a class of antibodies defined by the heavy chain constant region. Immunoglobulin genes include the kappa, lambda, alpha, gamma, delta, epsilon, and mu constant region genes. Light chains are classified as either kappa or lambda. Heavy chains are classified as gamma, mu, alpha, delta, or epsilon, which in turn define the isotype classes, IgG. IgM, IgA. IgD and IgE, respectively.

A “monoclonal antibody” refers to a clonal preparation of antibodies with a single binding specificity and affinity for a given epitope on an antigen. A “polyclonal antibody” refers to a preparation of antibodies that are raised against a single antigen, but with different binding specificities and affinities.

Antibodies include V_{H}-V_{L} dimers, including single chain antibodies (antibodies that exist as a single polypeptide chain), such as single chain Fv antibodies (sFv or scFv) in which a variable heavy and a variable light region are joined together (directly or through a peptide linker) to form a continuous polypeptide. The single chain Fv antibody is a covalently linked V_{H}-V_{L}, which may be expressed from a nucleic acid including V_{H}- and V_{L}-encoding sequences either joined directly or joined by a peptide-encoding linker (e.g., Huston, et al. Proc. Nat. Acad. Sci. USA, 85:5879-5883, 1988). While the V_{H} and V_{L} are connected to each as a single polypeptide chain, the V_{H} and V₁, domains associate non-covalently. Alternatively, the antibody can be another fragment. Other fragments can also be generated, e.g., using recombinant techniques, as soluble proteins or as fragments obtained from display methods. Antibodies can also include diantibodies and miniantibodies. Antibodies of the invention also include heavy chain dimers, such as antibodies from camelids.

As used herein, “V-region” refers to an antibody variable region domain comprising the segments of Framework 1, CDR1, Framework 2, CDR2, and Framework3, including CDR3 and Framework 4, which segments are added to the V-segment as a consequence of rearrangement of the heavy chain and light chain V-region genes during B-cell differentiation.

As used herein, “complementarity-determining region (CDR)” refers to the three hypervariable regions in each chain that interrupt the four “framework” regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

The sequences of the framework regions of different light or heavy chains are relatively conserved within a species. The framework region of an antibody, that is the combined framework regions of the constituent light and heavy chains, serves to position and align the CDRs in three dimensional space.

The amino acid sequences of the CDRs and framework regions can be determined using various well known definitions in the art, e.g., Kabat, Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press. Oxford, 141-172 1996).

As used herein, “chimeric antibody” refers to an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region, or portion thereof, having a different or altered antigen specificity; or with corresponding sequences from another species or from another antibody class or subclass.

As used herein, “humanized antibody” refers to an immunoglobulin molecule in which CDRs from a donor antibody are grafted onto human framework sequences. Humanized antibodies may also comprise residues of donor origin in the framework sequences. The humanized antibody can also comprise at least a portion of a human immunoglobulin constant region. Humanized antibodies may also comprise residues which are found neither in the recipient antibody nor in the imported CDR or framework sequences. Humanization can be performed using methods known in the art (e.g., Jones et al., Nature 321:522-525; 1986; Riechmann et al., Nature 332:323-327, 1988; Verhoeyen et al., Science 239:1534-1536, 1988); Presta, Curr. Op. Struct. Biol. 2:593-596, 1992; U.S. Pat. No. 4,816,567), including techniques such as “superhumanizing” antibodies (Tan et al., J. Immunol. 169: 1119, 2002) and “resurfacing” (e.g., Staclens et al., Mol. Immunol. 43: 1243, 2006; and Roguska et al., Proc. Natl. Acad. Sci USA 91: 969, 1994).

The antibody binds to an “epitope” on the antigen. The epitope is the specific antibody binding interaction site on the antigen, and can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope). The same is true for other types of target molecules that form three-dimensional structures.

The term “specifically bind” refers to a molecule (e.g., antibody or antibody fragment) that binds to a target with at least 2-fold greater affinity than non-target compounds, e.g., at least 4-fold, 5-fold, 6-fold, 7-fold, 8-fold, 9-fold, 10-fold, 20-fold, 25-fold, 50-fold, 100-fold, or 1000-fold greater affinity. For example, an antibody that specifically binds the receptor form of IL1RAP will typically bind to the receptor form of IL1RAP with at least a 2-fold greater affinity than a non-target molecules (e.g., the soluble form of IL1RAP, proteins related to IL1RAP, or other proteins).

The term “binds” with respect to a cell type (e.g., an antibody that binds lymphoma cells), typically indicates that an agent binds a majority of the cells in a pure population of those cells. For example, an antibody that binds a given cell type typically binds to at least ⅔ of the cells in a population of the indicated cells (e.g., 75, 80, 85, 90, 91, 92, 93, 94, 95, 96, 97, 98, 99, or 100%) if the antibody is in high enough concentration. In some cases, receptor-IL1RAP specific binding can be assayed by comparing binding of the antibody to a cell that expresses membrane-bound IL1RAP to binding (or lack thereof) of the antibody to a cell that does not express membrane-bound IL1RAP. One of skill will recognize that some variability will arise depending on the method and/or threshold of determining binding.

The term “cross-linked” with respect to an antibody refers to attachment of the antibody to a solid or semisolid matrix (e.g., sepharose, beads, culture plate), or to another protein or antibody. For example, the antibody can be multimerized to create an antibody complex with multiple (more than 2) antigen-binding sites. The antibody can be multimerized by expressing the antibody as a high-valency isotype (e.g., IgA or IgM, which typically form complexes of 2 or 5 antibodies, respectively). Antibody multimerization can also be carried out by using a cross-linker comprising a reactive group capable of linking proteins (e.g., carbodiimide, NHS esters, etc). Methods and compositions for cross-linking an antibody to a matrix are described, e.g., in the Abeam and New England Biolab catalogs and websites (available at abcam.com and neb.com). Cross-linker compounds with various reactive groups are described, e.g., in Thermo Fisher Scientific catalog and website (available at piercenet.com).

As used herein, a first antibody, or an antigen-binding portion thereof, “competes” for binding to a target with a second antibody, or an antigen-binding portion thereof, when binding of the second antibody with the target is detectably decreased in the presence of the first antibody compared to the binding of the second antibody in the absence of the first antibody. The alternative, where the binding of the first antibody to the target is also detectably decreased in the presence of the second antibody, can, but need not be the case. That is, a second antibody can inhibit the binding of a first antibody to the target without that first antibody inhibiting the binding of the second antibody to the target. However, where each antibody detectably inhibits the binding of the other antibody to its cognate epitope or ligand, whether to the same, greater, or lesser extent, the antibodies are said to “cross-compete” with each other for binding of their respective epitope(s). Both competing and cross-competing antibodies are encompassed by the present invention. The term “competitor” antibody can be applied to the first or second antibody as can be determined by one of skill in the art. In some cases, the presence of the competitor antibody (e.g., the first antibody) reduces binding of the second antibody to the target by at least 10%, e.g., 20%, 30%, 40%, 50%, 60%, 70%, 80%, or more, e.g., so that binding of the second antibody to target is undetectable in the presence of the first (competitor) antibody.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of benefit and/or side effects). Controls can be designed for in vitro applications. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any method known in the art for conjugating an antibody to the label may be employed, e.g., using methods described in Hermanson, Bioconjugate Techniques 1996, Academic Press, Inc., San Diego.

A “labeled” molecule (e.g., nucleic acid, protein, or antibody) is one that is bound, either covalently, through a linker or a chemical bond, or noncovalently, through ionic, van der Waals, electrostatic, or hydrogen bonds to a label such that the presence of the molecule may be detected by detecting the presence of the label bound to the molecule.

The term “diagnosis” refers to a relative probability that a disorder such as cancer or an inflammatory condition is present in the subject. Similarly, the term “prognosis” refers to a relative probability that a certain future outcome may occur in the subject. For example, in the context of the present invention, prognosis can refer to the likelihood that an individual will develop cancer, have recurrence, or the likely severity of the disease (e.g., severity of symptoms, rate of functional decline, survival, etc.). The terms are not intended to be absolute, as will be appreciated by any one of skill in the field of medical diagnostics.

“Biopsy” or “biological sample from a patient” as used herein refers to a sample obtained from a patient having, or suspected of having, a IL1RAP associated disorder. In some embodiments, the sample may be a tissue biopsy, such as needle biopsy, fine needle biopsy, surgical biopsy, etc. The sample can also be a blood sample or blood fraction, e.g., white blood cell fraction, serum, or plasma. The sample can comprise a tissue sample harboring a lesion or suspected lesion, although the biological sample may be also be derived from another site, e.g., a site of suspected metastasis, a lymph node, or from the blood. In some cases, the biological sample may also be from a region adjacent to the lesion or suspected lesion.

A “biological sample” can be obtained from a patient, e.g., a biopsy, from an animal, such as an animal model, or from cultured cells, e.g., a cell line or cells removed from a patient and grown in culture for observation. Biological samples include tissues and bodily fluids, e.g., blood, blood fractions, lymph, saliva, urine, feces, etc.

The terms “therapy,” “treatment,” and “amelioration” refer to any reduction in the severity of symptoms. In the case of treating cancer, treatment can refer to, e.g., reducing tumor size, number of cancer cells, growth rate, metastatic activity, reducing cell death of non-cancer cells, reduced nausea and other chemotherapy or radiotherapy side effects, etc. In the case of treating an inflammatory condition, the treatment can refer to, e.g., reducing blood levels of inflammatory cytokines, pain, swelling, recruitment of immune cells, etc. As used herein, the terms “treat” and “prevent” are not intended to be absolute terms. Treatment and prevention can refer to any delay in onset, amelioration of symptoms, improvement in patient survival, increase in survival time or rate, etc. Treatment and prevention can be complete (undetectable levels of neoplastic cells) or partial, such that fewer neoplastic cells are found in a patient than would have occurred without the present invention. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “effective amount,” “effective dose,” “therapeutically effective amount,” etc. refer to that amount of the therapeutic agent sufficient to ameliorate a disorder, as described above. For example, for the given parameter, a therapeutically effective amount will show an increase or decrease of therapeutic effect at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

“Inhibits” or “inhibition” as used herein, e.g., inhibition of characteristics such as cellular growth, proliferation, metabolic activity, viability, survival or division refers to a decrease in the characteristic relative to a control. In some cases, a compound of the present invention may inhibit one of the foregoing characteristics. For example, a cell treated with an antibody described herein may exhibit a decrease in one of the foregoing characteristics of approximately 1%, 2%, 3%, 4%, 5%, 6%, 7%, 8%, 9%, 10%, 12%, 15%, 20%, 25%, 30%, 35%, 40%, 45%, 50%, 60%, 70%, 75%, 80%, 85%, 90%, 95%, 97.5%, 99%, 99.5%, 99.9% or more as compared to an untreated cell. In some cases, a cell treated with a compound of the present invention may exhibit a growth, proliferation, metabolic activity, viability, survival or division that is less than about 1%, 2%, 3%, 4%, 5%, 6%, 7%, 8%, 9%, 10%, 12%, 15%, 20%, 25%, 30%, 35%, 40%, 45%, 50%, 60%, 70%, 75%, 80%, 85%, 90%, 95%, 97.5%, 99%, 99.5%, 99.9% of a control (e.g. untreated) cell.

“Inhibitors,” of IL1RAP activity refer to inhibitory molecules identified using in vitro and in vivo assays for IL1RAP activity, e.g., antagonists, and their homologs and mimetics. Inhibitors can, e.g., bind to or inactivate the activity of IL1RAP. Inhibitors include naturally occurring and synthetic antagonists (e.g., small chemical molecules, antibodies and the like that function as antagonists). Such assays for inhibitors include, e.g., applying putative inhibitor compounds to cells expressing IL1RAP and then determining the binding to the cells or measuring the functional activity of IL1RAP, as described herein. Functional activity of IL1RAP can include cellular proliferation or cell survival. Cells expressing IL1RAP that are treated with a potential inhibitor are compared to control samples without the inhibitor to examine the extent of effect. Control cells (untreated) are assigned a relative IL1RAP activity value of 100%.

Inhibition of IL1RAP is achieved when the IL1RAP activity value relative to the control is about 80%, or 70%, optionally 50%, 40%, 30%, 25%, 20%, 15%, 10%, 5%, 1% or lower. Inhibition of IL1RAP can provide a decrease in cellular proliferation or a decrease in cell survival. In other cases, inhibition can be detected by a decrease in binding or association of IL1RAP to a ligand or another protein, e.g. IL1R1, TOLLIP, MYD88, IRAK1 or IRAK2. In still other cases, inhibition can be detected by a decrease in interleukin-1-dependent activation of NF-kappa-B. Inhibition can also be detected by a decrease in the clonogenicity of, or decrease in the survival of, AML cells.

As used herein, the term “pharmaceutically acceptable” is used synonymously with physiologically acceptable and pharmacologically acceptable. A pharmaceutical composition will generally comprise agents for buffering and preservation in storage, and can include buffers and carriers for appropriate delivery, depending on the route of administration.

The terms “dose” and “dosage” are used interchangeably herein. A dose refers to the amount of active ingredient given to an individual at each administration. For the present invention, the dose can refer to the concentration of the antibody or associated components, e.g., the amount of therapeutic agent or dosage of radiolabel. The dose will vary depending on a number of factors, including frequency of administration; size and tolerance of the individual; severity of the condition; risk of side effects; the route of administration; and the imaging modality of the detectable moiety (if present). One of skill in the art will recognize that the dose can be modified depending on the above factors or based on therapeutic progress. The term “dosage form” refers to the particular format of the pharmaceutical, and depends on the route of administration. For example, a dosage form can be in a liquid, e.g., a saline solution for injection.

“Subject,” “patient,” “individual” and like terms are used interchangeably and refer to, except where indicated, mammals such as humans and non-human primates, as well as rabbits, rats, mice, goats, pigs, and other mammalian species. The term does not necessarily indicate that the subject has been diagnosed with a particular disease, but typically refers to an individual under medical supervision. A patient can be an individual that is seeking treatment, monitoring, adjustment or modification of an existing therapeutic regimen, etc. A “cancer patient” can refer to an individual that has been diagnosed with cancer, is currently following a therapeutic regimen, or is at risk of recurrence, e.g., after surgery to remove a tumor. In some embodiments, the cancer patient has been diagnosed with cancer and is a candidate for therapy. Cancer patients can include individuals that have not received treatment, are currently receiving treatment, have had surgery, and those that have discontinued treatment.

In the context of treating cancer, a subject in need of treatment can refer to an individual that has cancer or a pre-cancerous condition, has had cancer and is at risk of recurrence, is suspected of having cancer, is undergoing standard treatment for cancer, such as radiotherapy or chemotherapy, etc. Similarly, in the context of treating inflammation, a subject in need to treatment can refer to an individual that has an inflammatory condition (e.g., an allergic or immune response), is at risk of developing inflammation from a preexisting condition (e.g., allergies), or is at risk of developing inflammation due to exposure or likely exposure to an inflammatory or antigenic substance, e.g., due to travel.

“Cancer”. “tumor,” “transformed” and like terms include precancerous, neoplastic, transformed, and cancerous cells, and can refer to a solid tumor, or a non-solid cancer (see, e.g., Edge et al. AJCC Cancer Staging Manual (7^{th} ed. 2009); Cibas and Ducatman Cytology: Diagnostic principles and clinical correlates (3^{rd} ed. 2009)). Cancer includes both benign and malignant neoplasms (abnormal growth). “Transformation” refers to spontaneous or induced phenotypic changes, e.g., immortalization of cells, morphological changes, aberrant cell growth, reduced contact inhibition and anchorage, and/or malignancy (see, Freshney, Culture of Animal Cells a Manual of Basic Technique (3^{rd} ed. 1994)). Although transformation can arise from infection with a transforming virus and incorporation of new genomic DNA, or uptake of exogenous DNA, it can also arise spontaneously or following exposure to a carcinogen.

The term “cancer” can refer to carcinomas, sarcomas, adenocarcinomas, lymphomas, leukemias, solid and lymphoid cancers, etc. Examples of different types of cancer include, but are not limited to, lung cancer (e.g., non-small cell lung cancer or NSCLC), ovarian cancer, prostate cancer, colorectal cancer, liver cancer (i.e., hepatocarcinoma), renal cancer (i.e., renal cell carcinoma), bladder cancer, breast cancer, thyroid cancer, pleural cancer, pancreatic cancer, uterine cancer, cervical cancer, testicular cancer, anal cancer, pancreatic cancer, bile duct cancer, gastrointestinal carcinoid tumors, esophageal cancer, gall bladder cancer, appendix cancer, small intestine cancer, stomach (gastric) cancer, cancer of the central nervous system, skin cancer, choriocarcinoma; head and neck cancer, blood cancer, osteogenic sarcoma, fibrosarcoma, neuroblastoma, glioma, melanoma, B-cell lymphoma, non-Hodgkin's lymphoma. Burkitt's lymphoma, Small Cell lymphoma, Large Cell lymphoma, myelodysplastic syndromes (MDS), monocytic leukemia, myelogenous leukemia, acute lymphocytic leukemia, acute myelocytic leukemia (AML), chronic myeloid leukemia (CML), and multiple myeloma. In some embodiments, the compositions and methods of the present invention are useful for treating cancer.

A “cancer target” or “cancer marker” is a molecule that is differentially expressed or processed in cancer, e.g., on a cancer cell or in the cancer milieu. Exemplary cancer targets are cell surface proteins such as IL1RAP (also, e.g., cell adhesion molecules and receptors), intracellular receptors, hormones, and molecules such as proteases that are secreted by cells into the cancer milieu. Markers for specific cancers are known in the art, e.g., MUC1 expression on colon and colorectal cancers, bombesin receptors in lung cancer, and prostate specific membrane antigen (PSMA) on prostate cancer.

In some embodiments, the cancer target can be associated with a certain type of cancer cell, e.g., leukemia, myeloma, lymphoma, AML, CML, non-small cell lung cancer cells, prostate cancer, colorectal cancer, breast cancer or ovarian cancer. A cell type specific target is typically expressed at levels at least 2 fold greater in that cell type than in a reference population of cells. In some embodiments, the cell type specific marker is present at levels at least 3, 4, 5, 6, 7, 8, 9, 10 20, 50, 100, or 1000 fold higher than its average expression in a reference population. Thus, the target can be detected or measured to distinguish the cell type or types of interest from other cells.

A cancer stem cell (CSC) is a cell found in a tumor or blood cancer that can give rise to the cells that make up the bulk of the cancer. The CSC can also be self-renewing, similar to a normal (non-cancer) stem cell. CSCs can thus mediate metastasis by migrating to a non-tumor tissue in an individual and starting a “new” tumor. CSCs make up a very small percentage of any given cancer, depending on the stage that the cancer is detected. For example, the average frequency of CSCs in a sample of AML cells is believed to be about 1:10,000. Hematopoietic CSCs can be identified as CD34+, similar to normal hematopoietic stem cells (HSCs). Other CSC associated markers include CD44 (breast), CD133 (glial cancers), and Notch (e.g., myelomas and neuroblastoma).

An “inflammatory condition” refers to any inflammation in an individual, and can be transient (e.g., in response to exposure to a pathogen or allergen) or chronic. Inflammation is characterized by inflammatory cytokines such as IFN-gamma, IL-6, and TNF-alpha that recruit and activate macrophages and other leukocytes. In some cases, inflammation can develop into a chronic, harmful condition or autoimmune condition (e.g., MS, lupus, rheumatoid arthritis, Crohn's disease). Inflammation can be evident locally (e.g., at a localized site of infection or exposure) or systemically (e.g., atherosclerosis, high blood pressure).

### III. IL1RAP Associated Disorders

IL1RAP disorders can be treated, prevented, ameliorated, or mitigated with an antibody as described herein. IL1RAP associated disorders include cancers associated with elevated IL1RAP expression and inflammatory disorders associated with reduced IL1RAP expression, as described herein. The antibodies of the invention can be used for diagnosis and monitoring of these disorders, as well as targeted therapy, e.g., delivering an antibody or an antibody conjugated to a chemotherapeutic (or cytotoxic) agent specifically to an IL1RAP-expressing cell, such as a cancer cell while avoiding or reducing delivery to other cells. In some cases, the targeted therapy can comprise contacting an IL1RAP-expressing cell with an antibody, as described herein.

IL1RAP expression is associated with myelomas and other hematopoietic cell cancers, and carcinomas (e.g., carcinomas of the colon, ovary, liver, prostate, uterus, breast, and kidney). Examples of cancers that can be targeted using a receptor IL1RAP-specific antibody thus include hematopoietic cell cancers (e.g., B cell lymphoma, Burkitt's lymphoma, Hodgkin's lymphoma, non-Hodgkin's lymphoma, myelodysplastic syndrome (preleukemia), leukemias, and myelomas (e.g., acute myeloid leukemia (AML), chronic myeloid leukemia (CML), chronic myelomonocytic leukemia (CMML), multiple myeloma, plasmacytoma)). Additional IL1RAP expressing cancers that can be targeted using the presently disclosed antibodies include but are not limited to colon carcinoma, ovarian carcinoma, prostate carcinoma, breast carcinoma, renal carcinoma, liver carcinoma, and uterine carcinoma. Myelodysplastic syndromes (MDS) can also be targeted using a receptor IL1RAP-specific antibody.

In some cases, IL1RAP associated disorders that may be treated by the anti-receptor IL1RAP antibodies provided herein include joint, bone, and muscle diseases such as rheumatoid arthritis, ankylosing spondylitis, erosive osteoarthritis of the hand, recurrent multifocal osteomyelitis, traumatic knee injury, and relapsing polychondritis. In some cases, IL1RAP associated disorders that may be treated by the anti-receptor IL1RAP antibodies provided herein include hereditary systemic auto-inflammatory diseases such as familial Mediterranean fever, cryopyrin-associated periodic syndrome, TNF receptor-associated periodic syndrome, hyper-IgD syndrome, periodic fever, aphthous stomatitis, pharyngitis and adenitis, and deficiency of IL-1 receptor antagonist. In some cases, IL1RAP associated disorders that may be treated by the anti-receptor IL1RAP antibodies provided herein include systemic inflammatory diseases such as systemic juvenile idiopathic arthritis, adult-onset Still's disease, Schnitzler syndrome, Behçet's disease, synovitis acne pustulosis hyperostosis osteitis syndrome, and macrophage activation syndrome. In some cases, IL1RAP associated disorders that may be treated by the anti-receptor IL1RAP antibodies provided herein include common inflammatory diseases such as gout, pseudogout, type-2 diabetes, hidradenitis suppurativa, systolic heart failure, cardiac remodeling, dry eye syndrome, pustular psoriasis, and neutrophilic dermatoses. Further examples of IL1RAP associated disorders include diseases and conditions described in Dinarello et al. Nature Reviews Drug Discovery, Volume 11 Aug. 2012 p. 613 and supplementary materials provided therein.

### IV. Anti-Receptor IL1RAP Antibodies

Provided herein are anti-receptor IL1RAP antibodies (i.e., receptor IL1RAP-specific antibodies, anti-receptor IL1RAP) that specifically bind to the membrane-bound form of IL1RAP but do not substantially bind a soluble form of IL1RAP. In some embodiments, the anti-receptor IL1RAP antibodies provided herein specifically bind the membrane-bound form of IL1RAP but do not bind a soluble form of IL1RAP. Anti-receptor IL1RAP antibodies described herein can also inhibit growth of IL1RAP-expressing cells. In part in view of the data described herein, it is believed these anti-receptor IL1RAP antibodies can be used to inhibit cell growth of IL1RAP expressing cells (e.g., cancer cells) in the absence of a conjugated cytotoxic agent. It is also believed anti-receptor IL1RAP antibodies described herein also show complement dependent cytotoxicity (CDC) activity or antibody dependent cell-mediated cytotoxicity (ADCC). It is believed these anti-receptor IL1RAP antibodies can also be used to target IL1RAP expressing cells for destruction, e.g. in the absence of a conjugated cytotoxic agent. However, in some cases, cytotoxic agents may nevertheless be conjugated to anti-receptor IL1RAP antibodies of the invention.

Anti-receptor IL1RAP antibodies described herein have unique cell binding activities, such as those described in the examples herein. For example, anti-receptor IL1RAP antibodies can be used to target eosinophilic leukaemia cells (exemplified as EOL-1 cells). In some embodiments, anti-receptor IL1RAP antibodies can also bind AML, CML, or MDS cells, or solid tumor cells such as liver, kidney, lung, and brain tumor cells. In some embodiments, these antibodies can be used for detecting cancer cells that display an epitope that is targeted with high affinity by at least one of the anti-receptor IL1RAP antibodies disclosed herein. In some embodiments, those cancer cells can then be targeted for destruction with the same anti-receptor IL1RAP antibody. Such methods can include treating an individual having IL1RAP expressing cancer cells, e.g., as described herein, comprising administering the anti-receptor IL1RAP antibody to the individual.

In some cases, the anti-receptor IL1RAP antibodies have significant and surprising advantages over previously described anti-IL1RAP antibodies. For example, IL1RAP is expressed in a soluble and a membrane-bound form. In some cases, the soluble form is present in (e.g., secreted into) normal human serum. The inventor's have discovered that antibodies that bind both the soluble and membrane-bound form may therefore be blocked by the soluble form of IL1RAP and fail to accumulate on the surface of IL1RAP expressing cells. In contrast, it was discovered that anti-receptor IL1RAP antibodies provided herein are not blocked, or not substantially blocked, by naturally occurring soluble IL1RAP.

In some cases, anti-receptor antibodies described herein provide significant advantages over previously described anti-IL1RAP antibodies because the antibodies provided herein activate, or strongly activate antibody dependent cell-mediated cytotoxicity (ADCC) or complement dependent cytotoxicity (CDC), or a combination thereof. In some cases, receptor-specific IL1RAP antibodies described herein provide significantly greater activation of ADCC and/or CDC in comparison to anti-IL1RAP antibodies that are not receptor specific. For example, in some cases, the receptor-specific IL1RAP antibodies are capable of binding in large numbers to cells expressing membrane-bound IL1RAP, and are not, or not substantially, competed away from binding to these cells by soluble IL1RAP. In some cases, the antibodies provided herein are particularly advantageous for delivery of a cytotoxic or chemotherapeutic agent to cells expressing the receptor form of IL1RAP regardless of the presence of soluble IL1RAP.

In some embodiments, the anti-receptor IL1RAP antibody binds the same epitope as an antibody having the CDR sequences of 1C5, 1F5, 1E10, 2D8, 3A2, 12F1, 42E1, 4G9, or 4B6. In some embodiments, the anti-receptor IL1RAP antibody has 1, 2, or 3 heavy chain CDRs and/or 1, 2, or 3 light chain CDRs selected from the CDRs of antibody clones 1C5, 1F5, 1E10, 2D8, 3A2, 12F1, 42E1, 4G9, or 4B6. In some embodiments, the anti-receptor IL1RAP antibody has a light chain CDR sequence and heavy chain CDR sequence having up to 1, 2, 3, or 4 amino acid substitutions, additions, or deletions/CDR relative to one or more CDR sequences of antibody 1C5, 1F5, 1E10, 2D8, 3A, 12F1, 42E1, 4G9, or 4B62. In some embodiments, the light chain CDR sequences include up to 1, 2, 3, or 4 amino acid substitutions, additions or deletions/CDR relative to one or more light chain CDR sequences of the aforementioned anti-receptor IL1RAP antibodies. In some embodiments, the heavy chain CDR sequences include up to 1, 2, 3, or 4 amino acid substitutions, additions, or deletions/CDR relative to one or more heavy chain CDR sequences of the aforementioned anti-IL1RAP antibodies. In some embodiments, substitution, addition or deletion occurs in only 1, 2, 3, 4, or 5 CDRs of the 6 total CDRs. In some embodiments, the anti-receptor IL1RAP antibody has variable region sequences with at least 75%, 80%, 85%, 90%, 95% identity or higher to those of 1C5, 1F5, 1E10, 2D8, 3A2, 12F1, 42E1, 4G9, or 4B6.

In some cases, anti-receptor IL1RAP antibodies can comprise light chain variable region protein sequence SEQ ID NO: 5:

DVVMTQTPLSLPVSLGDQASISCRSSQSLVHINGNTYLHWYLQKPGQSPK
LLIYKVSNRFSGVPDRFSGSGSGTDFTLKISRVEAEDLGVYFCSQSTHVP
WTFGGGTKLEIK,

heavy chain variable region protein sequence SEQ ID NO: 6:

EVKLVESGGGLVKPGASLKLSCAASGFTFSNYGMSWVRQTSDKRL
EWVASISSGGGSTYYPDNVKGRFTISRENAKNTLYLQMSSLKSEDTALYY
CARVLPGRWAMDYWGQGSSVTVSS,

or both SEQ ID NO: 5 and SEQ ID NO: 6.

In other cases, anti-receptor IL1RAP antibodies can comprise a light chain variable region that is at least 85%, 90%, 95%, 97%, 98%, or 99% identical to SEQ ID NO: 5. Additionally, or in the alternative, anti-receptor IL1RAP antibodies can comprise a heavy chain variable region that is at least 85%, 90%, 95%, 97%, 98%, or 99% identical to SEQ ID NO: 6.

In some cases, anti-receptor IL1RAP antibodies can comprise one or more CDRs that are 85%, 90%, 95%, 97%, 98%, 99%, or 100% identical to the CDRs selected from the group consisting of

| | |
|---|---|
| 1F5 CDRH1, SEQ ID NO: 7: | NYGMS; |
| 1F5 CDRH2, SEQ ID NO: 8: | SISSGGGSTYYPDNVKG; |
| 1F5 CDRH3, SEQ ID NO: 9: | VLPGRWAMDY; |
| 1F5 CDRL1, SEQ ID NO: 10: | RSSQSLVHINGNTYLH; |
| 1F5 CDRL2, SEQ ID NO: 11: | KVSNRFS; |
| and | |
| 1F5 CDRL3, SEQ ID NO: 12: | SQSTHVPWT. |

In some cases, anti-receptor IL1RAP antibodies can comprise one or more CDRs that contain 1, 2, 3, or 4 amino acid substitutions or deletions as compared to a CDR selected from the group consisting of

| | |
|---|---|
| 1F5 CDRH1, SEQ ID NO: 7: | NYGMS; |
| 1F5 CDRH2, SEQ ID NO: 8: | SISSGGGSTYYPDNVKG; |
| 1F5 CDRH3, SEQ ID NO: 9: | VLPGRWAMDY; |
| 1F5 CDRL1, SEQ ID NO: 10: | RSSQSINHINGNTYLH; |
| 1F5 CDRL2, SEQ ID NO: 11: | KVSNRFS; |
| and | |
| 1F5 CDRL3, SEQ ID NO: 12: | SQSTHVPWT. |

In some cases, the anti-receptor IL1RAP antibodies can contain an amino acid sequence that is at least 85%, 90%, 95%, or 100% identical to the antibody light chain of antibody clone 12F1. Additionally, or in the alternative, the anti-receptor IL1RAP antibodies can contain an amino acid sequence that is at least 85%, 90%, 95%, or 100% identical to the antibody heavy chain of antibody clone 12F1. In some cases, the anti-receptor IL1RAP antibodies contain heavy and light chain complementarity determining regions of antibody clone 12F1. In some cases, the anti-receptor IL1RAP antibodies contain one, two, three, four, five, or six complementarity determining regions of antibody clone 12F1.

In some cases, anti-receptor IL1RAP antibodies contain one or more CDRs that are 85%, 90%, 95%, 97%, 98%, 99%, or 100% identical to the CDRs selected from the group consisting of

the CDRH1 of antibody clone 12F1;
the CDRH2 of antibody clone 12F1;
the CDRH3 of antibody clone 12F1;
the CDRL1 of antibody clone 12F1;
the CDRL2 of antibody clone 12F1; and
the CDRL3 of antibody clone 12F1.

In some cases, anti-receptor IL1RAP antibodies can comprise one or more CDRs that contain 1, 2, 3, or 4 amino acid substitutions or deletions as compared to a CDR selected from the group consisting of

the CDRH1 of antibody clone 12F1;
the CDRH2 of antibody clone 12F1:
the CDRH3 of antibody clone 12F1;
the CDRL1 of antibody clone 12F1;
the CDRL2 of antibody clone 12F1; and
the CDRL3 of antibody clone 12F1.

In some cases, the anti-receptor IL1RAP antibodies can contain an amino acid sequence that is at least 85%, 90%, 95%, or 100% identical to the antibody light chain variable region of antibody clone 42E1. Additionally, or in the alternative, the anti-receptor IL1RAP antibodies can contain an amino acid sequence that is at least 85%, 90%, 95%, or 100% identical to the antibody heavy chain variable region of antibody clone 42E1. In some cases, the anti-receptor IL1RAP antibodies contain heavy and light chain complementarity determining regions of antibody clone 42E1. In some cases, the anti-receptor IL1RAP antibodies contain one, two, three, four, five, or six complementarity determining regions of antibody clone 42E1.

In some cases, anti-receptor IL1RAP antibodies contain one or more CDRs that are 85%, 90%, 95%, 97%, 98%, 99%, or 100% identical to the CDRs selected from the group consisting of

the CDRH1 of antibody clone 42E1;
the CDRH2 of antibody clone 42E1;
the CDRH3 of antibody clone 42E1;
the CDRL1 of antibody clone 42E1;
the CDRL2 of antibody clone 42E1; and
the CDRL3 of antibody clone 42E1.

In some cases, anti-receptor IL1RAP antibodies can comprise one or more CDRs that contain 1, 2, 3, or 4 amino acid substitutions or deletions as compared to a CDR selected from the group consisting of

the CDRH1 of antibody clone 42E1;
the CDRH2 of antibody clone 42E1;
the CDRH3 of antibody clone 42E1;
the CDRL1 of antibody clone 42E1;
the CDRL2 of antibody clone 42E1; and
the CDRL3 of antibody clone 42E1.

In some cases, anti-receptor IL1RAP antibodies can comprise light chain variable region protein sequence SEQ ID NO: 13:

DVLMTQTPLSLPVSLGDQASISCRSSQSIVHSNGNTYLEWFLQKP
GQSPKLLIYTVSNRFSGVPDRFSGSGSGTDFTLKISRVEAEDLGVYYCFQ
GSHVPPTFGGGTKLEIK

heavy chain variable region protein sequence SEQ ID NO: 14:

QVQLQQSGTELVRPGASVKLSCKASGYTFTDYEMHWVKQTPVHGL
EWIGAIDPGTGGIAYNQKFKGKATLTADKSSSTAYMELRSLTSEDSAVYY
CTLYDYDLAYWGQGTLVTVSA,

or both SEQ ID NO: 13 and SEQ ID NO: 14.

In other cases, anti-receptor IL1RAP antibodies can comprise a light chain variable region that is at least 85%, 90%, 95%, 97%, 98%, or 99% identical to SEQ ID NO: 13. Additionally, or in the alternative, anti-receptor IL1RAP antibodies can comprise a heavy chain variable region that is at least 85%, 90%, 95%, 97%, 98%, or 99% identical to SEQ ID NO: 14.

In some cases, anti-receptor IL1RAP antibodies can comprise one or more CDRs that are 85%, 90%, 95%, 97%, 98%, 99%, or 100% identical to the CDRs selected from the group consisting of

| | |
|---|---|
| 4G9 CDRH1, SEQ ID NO: 15: | DYEMH; |
| 4G9 CDRH2, SEQ ID NO: 16: | AIDPGTGGIAYNQKFKG; |
| 4G9 CDRH3, SEQ ID NO: 17: | YDYDLAY; |
| 4G9 CDRL1, SEQ ID NO: 18: | RSSQSIVHSNGNTYLE; |
| 4G9 CDRL2, SEQ ID NO: 19: | TVSNRFS; |
| and | |
| 4G9 CDRL3, SEQ ID NO: 20: | FQGSHVPPT. |

In some cases, anti-receptor IL1RAP antibodies can comprise one or more CDRs that contain 1, 2, 3, or 4 amino acid substitutions or deletions as compared to a CDR selected from the group consisting of

| | |
|---|---|
| 4G9 CDRH1, SEQ ID NO: 15: | DYEMH; |
| 4G9 CDRH2, SEQ ID NO: 16: | AIDPGTGGIAYNQKFKG; |
| 4G9 CDRH3, SEQ ID NO: 17: | YDYDLAY; |
| 4G9 CDRL1, SEQ ID NO: 18: | RSSQSIVHSNGNTYLE; |
| 4G9 CDRL2, SEQ ID NO: 19: | TVSNRFS; |
| and | |
| 4G9 CDRL3, SEQ ID NO: 20: | FQGSHVPPT. |

In some cases, anti-receptor IL1RAP antibodies can comprise light chain variable region protein sequence SEQ ID NO: 21:

DIQMTQSPSSLSASLGERVSLTCRASQDIGSSLNWLQQEPDGTIK
RLIYATSSLDSGVPKRFSGSRSGSDYSLTISSLESEDFVDYYCLQYASSP
WTFGGGTKLEIK,

heavy chain variable region protein sequence SEQ ID NO: 22:

EVKLVESGGGLVKPGGSLKLSCAASGFTFRTYAMSWVRQTPEKRLEWVAS
ISSRGRIYYPDSVKGRFTISRDNARNILYLQMSSLRSEDTAMYYCARDGD
YGSSYDYWGQGTTLTVSS,

or both SEQ ID NO: 21 and SEQ ID NO: 22.

In other cases, anti-receptor IL1RAP antibodies can comprise a light chain variable region that is at least 85%, 90%, 95%, 97%, 98%, or 99% identical to SEQ ID NO: 21. Additionally, or in the alternative, anti-receptor IL1RAP antibodies can comprise a heavy chain variable region that is at least 85%, 90%, 95%, 97%, 98%, or 99% identical to SEQ ID NO: 22.

In some cases, anti-receptor IL1RAP antibodies can comprise one or more CDRs that are 85%, 90%, 95%, 97%, 98%, 99%, or 100% identical to the CDRs selected from the group consisting of

| | |
|---|---|
| 4B6 CDRH1, SEQ ID NO: 23: | TYAMS; |
| 4B6 CDRH2, SEQ ID NO: 24: | SISSRGRIYYPDSVKG; |
| 4B6 CDRH3, SEQ ID NO: 25: | DGDYOSSYDY; |
| 4B6 CDRL1, SEQ ID NO: 26: | RASQDIGSSLN; |
| 4B6 CDRL2, SEQ ID NO: 27: | ATSSLDS; |
| and | |
| 4B6 CDRL3, SEQ ID NO: 28: | LQYASSPWT. |

In some cases, anti-receptor IL1RAP antibodies can comprise one or more CDRs that contain 1, 2, 3, or 4 amino acid substitutions or deletions as compared to a CDR selected from the group consisting of

| | |
|---|---|
| 4B6 CDRH1, SEQ ID NO: 23: | TYAMS; |
| 4B6 CDRH2, SEQ ID NO: 24: | SISSRGRIYYPDSVKG; |
| 4B6 CDRH3, SEQ ID NO: 25: | DGDYGSSYDY; |
| 4B6 CDRL1, SEQ ID NO: 26: | RASQDIGSSLN; |
| 4B6 CDRL2, SEQ ID NO: 27: | ATSSLDS; |
| and | |
| 4B6 CDRL3, SEQ ID NO: 28: | LQYASSPWT. |

In an alternate embodiment, antibody 4B6 contains an antibody light chain variable region containing an amino acid sequence that is at least 85%, 90%, 95%, or 100% identical to at least 90 consecutive amino acids of SEQ ID NO: 48, or an antibody heavy chain variable region containing an amino acid sequence that is at least 85%, 90%, 95%, or 100% identical to at least 90 consecutive amino acids of SEQ ID NO: 50.

In an alternate embodiment, antibody 4B6 contains an antibody light chain variable region containing an amino acid sequence that is at least 85%, 90%, 95%, or 100% identical to at least 90 consecutive amino acids of SEQ ID NO: 44, or an antibody heavy chain variable region containing an amino acid sequence that is at least 85%, 90%, 95%, or 100% identical to at least 90 consecutive amino acids of SEQ ID NO: 46.

In an alternate embodiment, antibody 4B6 contains an antibody light chain variable region containing an amino acid sequence that is at least 85%, 90%, 95%, or 100% identical to at least 90 consecutive amino acids of SEQ ID NO: 48, or an antibody heavy chain variable region containing an amino acid sequence that is at least 85%, 90%, 95%, or 100% identical to at least 90 consecutive amino acids of SEQ ID NO: 46.

In an alternate embodiment, antibody 4B6 contains an antibody light chain variable region containing an amino acid sequence that is at least 85%, 90%, 95%, or 100% identical to at least 90 consecutive amino acids of SEQ ID NO: 44, or an antibody heavy chain variable region containing an amino acid sequence that is at least 85%, 90%, 95%, or 100% identical to at least 90 consecutive amino acids of SEQ ID NO: 50.

In an alternate embodiment, anti-receptor IL1RAP antibodies can comprise one or more CDRs that are 85%, 90%, 95%, 97%, 98%, 99%, or 100% identical to the CDRs selected from the group consisting of

| | |
|---|---|
| 4B6 CDRH1, SEQ ID NO: 51: | DYYMH; |
| 4B6 CDRH2, SEQ ID NO: 52: | WIDPENGNTIYDPKFQG; |
| 4B6 CDRH3, SEQ ID NO: 53: | YYGHLYWYFDV; |
| 4B6 CDRL1, SEQ ID NO: 54: | KASQDVSTAVA; |
| 4B6 CDRL2, SEQ ID NO: 55: | SASYRYT; |
| and | |
| 4B6 CDRL3, SEQ ID NO: 56: | QQHYSSPLT. |

In an alternate embodiment, anti-receptor IL1RAP antibodies can comprise one or more CDRs that contain 1, 2, 3, or 4 amino acid substitutions or deletions as compared to a CDR selected from the group consisting of

| | |
|---|---|
| 4B6 CDRH1, SEQ ID NO: 51: | DYYMH; |
| 4B6 CDRH2, SEQ ID NO: 52: | WIDPENGNTIYDPKFQG; |
| 4B6 CDRH3, SEQ ID NO: 53: | YYGHLYWYFDV; |
| 4B6 CDRL1, SEQ ID NO: 54: | KASQDVSTAVA; |
| 4B6 CDRL2, SEQ ID NO: 55: | SASYRYT; |
| and | |
| 4B6 CDRL3, SEQ ID NO: 56: | QQHYSSPLT. |

In an alternate embodiment, anti-receptor IL1RAP antibodies can comprise one or more CDRs that are 85%, 90%, 95%, 97%, 98%, 99%, or 100% identical to the CDRs selected from the group consisting of

| | |
|---|---|
| 4B6 CDRH1, SEQ ID NO: 23: | TYAMS; |
| 4B6 CDRH2, SEQ ID NO: 24: | SISSRGRIYYPDSVKG; |
| 4B6 CDRH3, SEQ ID NO: 25: | DGDYGSSYDY; |
| 4B6 CDRL1, SEQ ID NO: 54: | KASQDVSTAVA; |
| 4B6 CDRL2, SEQ ID NO: 55: | SASYRYT; |
| and | |
| 4B6 CDRL3, SEQ ID NO: 56: | QQHYSSPLT. |

In an alternate embodiment, anti-receptor IL1RAP antibodies can comprise one or more CDRs that contain 1, 2, 3, or 4 amino acid substitutions or deletions as compared to a CDR selected from the group consisting of

| | |
|---|---|
| 4B6 CDRH1, SEQ ID NO: 23: | TYAMS; |
| 4B6 CDRH2, SEQ ID NO: 24: | SISSRGRTYYPDSVKG; |
| 4B6 CDRH3, SEQ ID NO: 25: | DGDYGSSYDY; |
| 4B6 CDRL1, SEQ ID NO: 54: | KASQDVSTAVA; |
| 4B6 CDRL2, SEQ ID NO: 55: | SASYRYT; |
| and | |
| 4B6 CDRL3, SEQ ID NO: 56: | QQHYSSPLT. |

In an alternate embodiment, anti-receptor IL1RAP antibodies can comprise one or more CDRs that are 85%, 90%, 95%, 97%, 98%, 99%, or 100% identical to the CDRs selected from the group consisting of

| | |
|---|---|
| 4B6 CDRH1, SEQ ID NO: 51: | DYYMH; |
| 4B6 CDRH2, SEQ ID NO: 52: | WIDPENGNTIYDPKFQG; |
| 4B6 CDRH3, SEQ ID NO: 53: | YYGHLYWYFDV; |
| 4B6 CDRL1, SEQ ID NO: 26: | RASQDIGSSLN; |
| 4B6 CDRL2, SEQ ID NO: 27: | ATSSLDS; |
| and | |
| 4B6 CDRL3, SEQ ID NO: 28: | LQYASSPWT. |

In an alternate embodiment, anti-receptor IL1RAP antibodies can comprise one or more CDRs that contain 1, 2, 3, or 4 amino acid substitutions or deletions as compared to a CDR selected from the group consisting of

| | |
|---|---|
| 4B6 CDRH1, SEQ ID NO: 51: | DYYMH |
| 4B6 CDRH2, SEQ ID NO: 52: | WIDPENGNTIYDPKFQG; |
| 4B6 CDRH3, SEQ ID NO: 53: | YYGHLYWYFDV; |
| 4B6 CDRL1, SEQ ID NO: 26: | RASQDIGSSLN; |
| 4B6 CDRL2, SEQ ID NO: 27: | ATSSLDS; |
| and | |
| 4B6 CDRL3, SEQ ID NO: 28: | LQYASSPWT. |

In some embodiments, anti-receptor IL1RAP antibodies can comprise a CDRL1 containing amino acid sequence SEQ ID NO: 51, a CDRH2 containing amino acid sequence SEQ ID NO: 52, and a CDRH3 containing amino acid sequence SEQ ID NO: 53.

In some embodiments, anti-receptor IL1RAP antibodies can comprise a CDRL1 containing amino acid sequence SEQ ID NO: 54, a CDRL2 containing amino acid sequence SEQ ID NO: 55, and a CDRL3 containing amino acid sequence SEQ ID NO: 56.

In some cases, anti-receptor IL1RAP antibodies can comprise one or more CDRs that are 85%, 90%, 95%, 97%, 98%, 99%, or 100% identical to the CDRs selected from the group consisting of

| | |
|---|---|
| 1F5 CDRH1, SEQ ID NO: 29: | GFTFSNYG; |
| 1F5 CDRH2, SEQ ID NO: 30: | ISSGGGST; |
| 1F5 CDRH3, SEQ ID NO: 31: | ARVLPGRWAMDY; |
| 1F5 CDRL1, SEQ ID NO: 32: | QSLVHINGNTY; |
| 1F5 CDRL2, SEQ ID NO: 33: | KVS; |
| and | |
| 1F5 CDRL3, SEQ ID NO: 12: | SQSTHVPWT. |

In some cases, anti-receptor IL1RAP antibodies can comprise one or more CDRs that contain 1, 2, 3, or 4 amino acid substitutions or deletions as compared to a CDR selected from the group consisting of

| | |
|---|---|
| 1F5 CDRH1, SEQ ID NO: 29: | GFTFSNYG; |
| 1F5 CDRH2, SEQ ID NO: 30: | ISSGGGST; |
| 1F5 CDRH3, SEQ ID NO: 31: | ARVLPGRWAMDY; |
| 1F5 CDRL1, SEQ ID NO: 32: | QSLVHINGNTY; |
| 1F5 CDRL2, SEQ ID NO: 33: | KVS; |
| and | |
| 1F5 CDRL3, SEQ ID NO: 12: | SQSTHVPWT. |

Any of the antibodies described herein can be a chimeric antibody or a humanized antibody. In some embodiments, the antibody is an anti-receptor IL1RAP-binding antibody fragment, e.g., an Fab. In some embodiments, the anti-receptor IL1RAP antibody is labeled with a detectable agent, e.g., as described below. In some embodiments, the anti-receptor IL1RAP antibody is attached to a therapeutic agent, e.g., a chemotherapeutic or cytotoxic agent as described below.

In some embodiments, the antibody also has at least one activity selected from
Binding to the receptor form of IL1RAP with a K_{D} of 100 μM or lower (e.g., 1-10 μM, 0.1-10M, 10-50 μM, about 20 μM, etc.);
Binding to the receptor form of IL1RAP with a K_{D} of 100 nM or lower (e.g., 1-10 nM, 0.1-10 nM, 10-50 nM, about 20 nM, etc.);
Reducing cell growth of IL1RAP-expressing cells, compared to cell growth in the absence of the antibody; and
Reducing cell growth of IL1RAP-expressing cells when cross-linked or immobilized (e.g., on a solid or semisolid matrix), compared to cell growth in the absence of the antibody.

In some embodiments, the anti-receptor IL1RAP antibody binds to IL1RAP from a human. In some embodiments, the anti-receptor IL1RAP antibody binds to IL1RAP from a rodent (mouse or rat).

A. Methods of Making Antibodies

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778. U.S. Pat. No. 4,816,567) can be adapted to produce antibodies to polypeptides of this invention. Also, transgenic mice, or other organisms such as other mammals, can be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Antibodies can be produced using any number of expression systems, including prokaryotic and eukaryotic expression systems. In some embodiments, the expression system is a mammalian cell expression, such as a hybridoma, or a CHO cell expression system. Many such systems are widely available from commercial suppliers. In embodiments in which an antibody comprises both a V_{H} and V_{L} region, the V_{H} and V_{L} regions may be expressed using a single vector, e.g., in a di-cistronic expression unit, or under the control of different promoters. In other embodiments, the V_{H} and V_{L} region may be expressed using separate vectors. A V_{H} or V_{L} region as described herein may optionally comprise a methionine at the N-terminus.

An antibody of the invention can also be produced in various formats, including as a Fab, a Fab′, a F(ab′)₂, a scFv, or a dAB. The antibody fragments can be obtained by a variety of methods, including, digestion of an intact antibody with an enzyme, such as pepsin (to generate (Fab′)₂ fragments) or papain (to generate Fab fragments); or de novo synthesis. Antibody fragments can also be synthesized using recombinant DNA methodology. In some embodiments, the anti-receptor IL1RAP antibody comprises F(ab′)₂ fragments that specifically bind the membrane-bound form of IL1RAP. An antibody of the invention can also include a human constant region. See, e.g., Fundamental Immunology (Paul ed., 4d ed. 1999): Bird, et al., Science 242:423 (1988); and Huston, et al., Proc. Natl. Acad. Sci. USA 85:5879 (1988).

Methods for humanizing or primatizing non-human antibodies are also known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

In some cases, the antibody or antibody fragment can be conjugated to another molecule, e.g., polyethylene glycol (PEGylation) or serum albumin, to provide an extended half-life in vivo. Examples of PEGylation of antibody fragments are provided in Knight et al. Platelets 15:409, 2004 (for abciximab); Pedley et al., Br. J. Cancer 70:1126, 1994 (for an anti-CEA antibody); Chapman et al., Nature Biotech. 17:780, 1999; and Humphreys, et al., Protein Eng. Des. 20: 227, 2007). The antibody or antibody fragment can also be labeled, or conjugated to a therapeutic agent as described below.

### Binding Affinity

The specificity of the binding can be defined in terms of the comparative dissociation constants (K_{d}) of the antibody (or other targeting moiety) for target, as compared to the dissociation constant with respect to the antibody and other materials in the environment or unrelated molecules in general. Typically, the K_{d} for the antibody with respect to the unrelated material will be at least 2-fold; 3-fold; 4-fold; 5-fold; 10-fold; 20-fold; 25-fold; 50-fold; 100-fold; 200-fold; 250-fold; 500-fold; 750-fold; 1,000-fold; 10,000-fold; 100,000-fold; 1,000,000-fold or more higher than the K_{d} with respect to the target. As used herein, a higher K_{d} is a K_{d} that describes a lower affinity interaction. Conversely a better or lower K_{d} is a K_{d} that describes a higher affinity interaction or tighter binding. By way of example only, the K_{d} for an antibody specifically binding to a target may be femtomolar, picomolar, nanomolar, or micromolar and the K_{d} for the antibody binding to unrelated material may be millimolar or higher. Accordingly, an antibody can bind the target molecule with an affinity that is 2-fold; 3-fold; 4-fold; 5-fold; 10-fold; 20-fold; 25-fold; 50-fold; 100-fold; 200-fold; 250-fold; 500-fold; 750-fold; 1,000-fold; 10,000-fold; 100,000-fold; 1,000,000-fold or more greater than the affinity for unrelated material. In some cases, the K_{d} for the antibody binding to unrelated materials may be undetectable.

In some cases, the specificity of binding can be defined in terms of the comparative dissociation constants (K_{d}) of the antibody (or other targeting moiety) for target (i.e., the receptor form of IL1RAP), as compared to the dissociation constant with respect to the antibody (or other targeting moiety) and the soluble form of IL1RAP. For example, the K_{d} for the antibody with respect to the soluble IL1RAP can be at least 2-fold, 3-fold, 4-fold, 5-fold, 10-fold, 20-fold, 50-fold, 100-fold, 200-fold or more higher (i.e., lower affinity or worse binding) than the K_{d} with respect to the receptor form of IL1RAP. Accordingly, the antibody can bind the receptor form of IL1RAP with an affinity that is 2-fold; 3-fold; 4-fold; 5-fold; 10-fold; 20-fold; 25-fold; 50-fold; 100-fold; 200-fold; 250-fold; 500-fold; 750-fold; 1,000-fold; 10,000-fold; 100,000-fold; 1,000,000-fold or more greater than the affinity for the soluble form of IL1RAP.

In some cases, anti-receptor IL1RAP antibodies of the invention bind the receptor form of IL1RAP but do not bind the soluble form of IL1RAP. For example, the antibodies exhibit no detectable or discernible affinity to soluble IL1RAP. In other cases, the antibodies do not substantially bind to soluble IL1RAP. For example, the antibodies do not exhibit detectable binding to soluble IL1RAP greater than background. As another example, antibodies of the invention that bind the receptor form of IL1RAP but do not substantially bind to the soluble form can exhibit a K_{d} for binding to the soluble form that is 20-fold, 50-fold, 100-fold; 1,000-fold, 10,000-fold, 1,000,000-fold or greater than the K_{d} for binding to the receptor form of IL1RAP. Antibodies of the invention that bind the receptor form of IL1RAP but do not substantially bind to the soluble form can also exhibit an affinity for the receptor form that is 20-fold, 50-fold, 100-fold; 1,000-fold, 10,000-fold, 1,000,000-fold or greater than the affinity for binding to the soluble form of IL1RAP. In some cases, the anti-receptor IL1RAP antibodies of the invention can bind the receptor form of IL1RAP with a femtomolar, picomolar, or micromolar K_{d} and exhibit millimolar or no detectable binding, e.g. by ELISA, FACS, surface plasmon resonance, etc., to the soluble form of IL1RAP.

The desired affinity for an antibody, e.g., high (pM to low nM), medium (low nM to 100 nM), or low (about 100 nM or higher), may differ depending upon whether it is being used as a diagnostic or therapeutic. Without being limited to theory, in one example, an antibody with medium affinity may be more successful in localizing to a tumor as compared to one with a high affinity. Thus, antibodies having different affinities can be used for diagnostic and therapeutic applications.

A targeting moiety will typically bind with a K_{d} of less than about 1000 nM, e.g., less than 250, 100, 50, 20, 10, 5, 1, 0.5, 0.1, 0.05, 0.01 or lower nM. In some embodiments, the K_{d} of the affinity agent is less than 15, 10, 5, 1, 0.5, 0.1, or 0.01 nM. In some embodiments, the K_{d} is 1-100 nM, 0.1-50 nM, 0.1-10 nM, 0.01-1, 0.01-0.1, or 1-20 nM. The value of the dissociation constant (K_{d}) can be determined by well-known methods, and can be computed even for complex mixtures by methods as disclosed, e.g., in Caceci et al., Byte (1984) 9:340-362.

Affinity of an antibody, or any targeting agent, for a target can be determined according to methods known in the art, e.g., as reviewed in Ernst et al. Determination of Equilibrium Dissociation Constants, Therapeutic Monoclonal Antibodies (Wiley & Sons ed. 2009).

Quantitative ELISA, and similar array-based affinity methods can be used. ELISA (Enzyme linked immunosorbent signaling assay) is an antibody-based method. In some cases, an antibody specific for target of interest is affixed to a substrate, and contacted with a sample suspected of containing the target. The surface is then washed to remove unbound substances. Target binding can be detected in a variety of ways, e.g., using a second step with a labeled antibody, direct labeling of the target, or labeling of the primary antibody with a label that is detectable upon antigen binding. In some cases, the antigen is affixed to the substrate (e.g., using a substrate with high affinity for proteins, or a Strepavidin-biotin interaction) and detected using a labeled antibody (or other targeting moiety). Several permutations of the original ELISA methods have been developed and are known in the art (see Lequin (2005) Clin. Chem. 51:2415-18 for a review).

The K_{d}, Kₒₙ, and K_{off} can also be determined using, for example, surface plasmon resonance (SPR), e.g., as measured by using a Biacore T100 system. SPR techniques are reviewed, e.g., in Hahnfeld et al. Determination of Kinetic Data Using SPR Biosensors, Molecular Diagnosis of Infectious Diseases (2004). In a typical SPR experiment, one interactant (target or targeting agent) is immobilized on an SPR-active, gold-coated glass slide in a flow cell, and a sample containing the other interactant is introduced to flow across the surface. When light of a given frequency is shined on the surface, the changes to the optical reflectivity of the gold indicate binding, and the kinetics of binding.

Binding affinity can also be determined by anchoring a biotinylated interactant to a streptaviden (SA) sensor chip. The other interactant is then contacted with the chip and detected, e.g., as described in Abdessamad et al. (2002) Nuc. Acids Res. 30:e45.

### V. Diagnostic Applications

The antibodies described herein specifically bind the receptor form of IL1RAP and IL1RAP-expressing cells. The receptor IL1RAP-specific antibodies can thus be used for in vitro and in vivo diagnostic assays to detect IL1RAP-expressing cells (e.g., CSCs; certain solid tumor cells; hematopoietic cancer cells; cells of the joint, bone or muscle; or cells involved in inflammatory diseases as indicated herein). For example, a sample (e.g., blood sample or tissue biopsy) can be obtained from a patient and contacted with a receptor IL1RAP antibody, and the presence of a IL1RAP expressing cell in the patient sample can be determined by detecting antibody binding. Antibody binding can be detected directly (e.g., where the antibody itself is labeled) or by using a second detection agent, such as a secondary antibody. The detectable label can be associated with an antibody of the invention, either directly, or indirectly, e.g., via a chelator or linker. In some cases, antibodies of the invention specific for the receptor form of IL1RAP but not the soluble form are useful for the detection or capture of IL1RAP expressing cells from blood or other bodily fluids that contain soluble IL1RAP.

In some embodiments, the anti-receptor IL1RAP antibody is contacted with a biological sample from an individual having or suspected of having an IL1RAP associated disorder, and antibody binding to a cell in the sample is determined, wherein higher or lower than normal antibody binding indicates that the individual has an IL1RAP associated disorder. In some embodiments, the biological sample is a blood sample or blood fraction (e.g., serum, plasma, platelets, red blood cells, white blood cells). In some embodiments, the biological sample is a tissue sample (biopsy), e.g., from a suspected tumor site, or from a tissue that is known to be affected, e.g., to determine the boundaries of a known tumor. In some embodiments, the biological sample is obtained from a site of inflammation.

Biopsies are typically performed to obtain samples from tissues, i.e., non-fluid cell types. The biopsy technique applied will depend on the tissue type to be evaluated (e.g., breast, skin, colon, prostate, kidney, lung, bladder, lymph node, liver, bone marrow, airway or lung). In the case of a cancer the technique will also depend on the size and type of the tumor (e.g., solid, suspended, or blood), among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy” of the tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70, and throughout Part V.

Any method of detecting antibody binding to a cell in a sample can be used for the present diagnostic assays. Methods of detecting antibody binding are well known in the art, e.g., flow cytometry, fluorescent microscopy, ELISAs, etc. In some embodiments, the method comprises preparing the biological sample for detection prior to the determining step. For example, a subpopulation of cells (e.g., white blood cells) can be separated from the rest of the sample from the individual (e.g., other blood components) or cells in a tissue can be suspended for easier detection.

In some embodiments, the percentage of IL1RAP-expressing cells in the sample is determined and compared to a control, e.g., a sample from an individual or group of individuals that are known to have an IL1RAP associated disorder (positive control) or from an individual or group of individuals that are known not to have an IL1RAP associated disorder (normal, non-disease, or negative control). In some embodiments, the control is a standard range of IL1RAP expression established for a given tissue. A higher or lower than normal percentage of IL1RAP expressing cells, or higher or lower expression level, indicates that the individual has an IL1RAP associated disorder.

In some embodiments, a labeled anti-receptor IL1RAP antibody can be provided (administered) to an individual to determine the applicability of an intended therapy. For example, a labeled antibody may be used to detect IL1RAP density within a diseased area, where the density is typically high relative to non-diseased tissue. A labeled antibody can also indicate that the diseased area is accessible for therapy. Patients can thus be selected for therapy based on imaging results. Anatomical characterization, such as determining the precise boundaries of a cancer, can be accomplished using standard imaging techniques (e.g., CT scanning, MRI, PET scanning, etc.).

In some embodiments, labeled antibodies specific for receptor IL1RAP as described herein can be further associated with a therapeutic compound, e.g., to form a “theranostic” composition. For example, an anti-receptor IL1RAP antibody described herein can be linked (directly or indirectly) to both a detectable label and a therapeutic agent, e.g., a cytotoxic agent to kill IL1RAP-expressing cancer cells. In some embodiments, a labeled antibody specific for the receptor form of IL1RAP is used for diagnosis and/or localization of an IL1RAP expressing cancer cell, and the IL1RAP expressing cancer cell is then targeted with a separate therapeutic antibody specific for the receptor form of IL1RAP. In some embodiments, the diagnostic antibody that is specific for the receptor form of IL1RAP is one that is not internalized into IL1RAP expressing cells at a high rate or percentage. In some embodiments, the therapeutic antibody specific for the receptor form of IL1RAP is an antibody that inhibits proliferation of IL1RAP expressing cells upon crosslinking or multimerization.

A. Labels

A diagnostic agent comprising an anti-receptor IL1RAP antibody can include any diagnostic agent known in the art, as provided, for example, in the following references: Armstrong et al., Diagnostic Imaging, 5^{th} Ed., Blackwell Publishing (2004); Torchilin, V. P., Ed., Targeted Delivery of Imaging Agents, CRC Press (1995); Vallabhajosula, S., Molecular Imaging: Radiopharmaceuticals for PET and SPECT, Springer (2009). A diagnostic agent can be detected by a variety of ways, including as an agent providing and/or enhancing a detectable signal. Detectable signals include, but are not limited to, gamma-emitting, radioactive, echogenic, optical, fluorescent, absorptive, magnetic, or tomography signals. Techniques for imaging the diagnostic agent can include, but are not limited to, single photon emission computed tomography (SPECT), magnetic resonance imaging (MRI), optical imaging, positron emission tomography (PET), computed tomography (CT), x-ray imaging, gamma ray imaging, and the like.

The terms “detectable agent,” “detectable moiety,” “label,” “imaging agent,” and like terms are used synonymously herein.

In some embodiments, the label can include optical agents such as fluorescent agents, phosphorescent agents, chemiluminescent agents, and the like. Numerous agents (e.g., dyes, probes, labels, or indicators) are known in the art and can be used in the present invention. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof. For example, fluorescent agents can include but are not limited to cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulcnes, azaazulenes, triphenyl methane dyes, indoles, benzoindoles, indocarbocyanines, benzoindocarbocyanines, and BODIPY™ derivatives. Fluorescent dyes are discussed, for example, in U.S. Pat. No. 4,452,720, U.S. Pat. No. 5,227,487, and U.S. Pat. No. 5,543,295.

The label can also be a radioisotope, e.g., radionuclides that emit gamma rays, positrons, beta and alpha particles, and X-rays. Suitable radionuclides include but are not limited to ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga, ³H, ¹⁶⁶Ho, ¹²³I, ¹²⁴I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y and ⁹⁰Y. In some embodiments, radioactive agents can include ¹¹¹In-DTPA, ^{99m}Tc(CO)₃-DTPA, ^{99m}Tc(CO)₃-ENPy2, ^{62/64/67} Cu-TETA, ^{99m}Tc(CO)₃-IDA, and ^{99m}Tc(CO)₃triamines (cyclic or linear). In some embodiments, the agents can include DOTA and its various analogs with ¹¹¹In, ¹⁷⁷Lu, ¹⁵³Sm, ^{88/90}Y, ^{62/64/67}Cu, or ^{67/68}Ga. In some embodiments, a nanoparticle can be labeled by incorporation of lipids attached to chelates, such as DTPA-lipid, as provided in the following references: Phillips et al., Wiley Interdisciplinary Reviews: Nanomedicine and Nanobiotechnology, 1(1): 69-83 (2008); Torchilin, V. P. & Weissig, V., Eds. Liposomes 2nd Ed.: Oxford Univ. Press (2003); Elbayoumi. T. A. & Torchilin, V. P., Eur. J. Nucl. Med. Mol. Imaging 33:1196-1205 (2006); Mougin-Degraef, M. et al., Int'l J. Pharmaceutics 344:110-117 (2007).

In some embodiments, the diagnostic agent can be associated with a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Secondary binding ligands include, e.g., biotin and avidin or streptavidin compounds as known in the art.

In some embodiments, the labeled antibody can be further associated to a composition that improves stability in vivo, e.g. PEG or a nanoparticle such as a liposome, as described in more detail below.

B. Methods of Labeling

Techniques for conjugating detectable and therapeutic agents to antibodies are well known (see. e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies'84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

Typically, the antibody is attached to detectable moiety in an area that does not interfere with binding to the epitope. Thus in some cases, the detectable moiety is attached to the constant region, or outside the CDRs in the variable region. One of skill in the art will recognize that the optimal position for attachment may be located elsewhere on the antibody, so the position of the detectable moiety can be adjusted accordingly. In some embodiments, the ability of the antibody to associate with the epitope is compared before and after attachment to the detectable moiety to ensure that the attachment does not unduly disrupt binding.

In some embodiments, the antibody can be associated with an additional targeting moiety. For example, an antibody fragment, peptide, or aptamer that binds a different site on the target molecule or target cell can be conjugated to the antibody to optimize target binding, e.g., to a cancer cell.

### VI. Therapeutic Applications

IL1RAP is aberrantly expressed in a number of disease states, and the IL1RAP-expressing cells in such conditions can be targeted using the antibodies described herein that are specific for the receptor form of IL1RAP. For example, IL1RAP expression is elevated on cancer cells (e.g., B cell lymphoma, AML cells, and solid tumor cells described herein); CSCs (e.g., myeloid CSCs); and cells associated with the IL1RAP associated disorders provided herein. IL1RAP is not significantly expressed on normal hematopoietic stem cells (HSCs). As noted above, a therapeutic composition comprising an anti-receptor IL1RAP antibody can further include a detectable label to form a theranostic composition, e.g., for detection and localization of IL1RAP expressing cells, and monitoring of therapeutic effect.

A. Chemotherapeutic and Cytotoxic Agents

As demonstrated herein, anti-receptor IL1RAP antibodies can inhibit cancer cell growth (proliferation), and thus can be considered chemotherapeutic agents. The following disclosure provides examples of additional chemotherapeutic and cytotoxic agents that can be linked to an anti-receptor IL1RAP antibody for delivery to IL1RAP-expressing cells.

A chemotherapeutic (anti-cancer) agent can be any agent capable of reducing cancer growth, interfering with cancer cell replication, directly or indirectly killing cancer cells, reducing metastasis, reducing tumor blood supply, etc. Chemotherapeutic agents thus include cytotoxic agents. Cytotoxic agents include but are not limited to saporin, taxanes, vinca alkaloids, anthracycline, and platinum-based agents. Classes of chemotherapeutic agents include but are not limited to alkylating agents, antimetabolites, e.g, methotrexate, plant alkaloids, e.g., vincristine, and antitumor antibiotics such as anthracyclines, e.g., doxorubicin as well as miscellaneous drugs that do not fall in to a particular class such as hydroxyurea. Platinum-based drugs, exemplified by cisplatin and oxaliplatin, represent a major class of chemotherapeutics. These drugs bind to DNA and interfere with replication. Taxanes, exemplified by taxol, represent another major class of chemotherapeutics. These compounds act by interfering with cytoskeletal and spindle formation to inhibit cell division, and thereby prevent growth of rapidly dividing cancer cells. Other chemotherapeutic drugs include hormonal therapy. Chemotherapeutics also include agents that inhibit tubulin assembly or polymerization such as maytansine, mertansine, and auristatin. Chemotherapeutic agents also include DNA damage agents such as calicheamicin.

Chemotherapeutics currently used for treating myeloma include bortezomib, lenalidomide, and thalidomide. Additional therapeutic agents that can be administered to myeloma patients include bisphosphonates (to prevent bone fractures) and erythropoietin (to reduce anemia).

More than one therapeutic agent can be combined, either in the same composition, or in separate compositions. The therapeutic agent(s) can also be combined with additional therapeutic agents as appropriate for the particular individual. Common therapeutic agents provided to cancer patients include medications to address pain, nausea, anemia, infection, inflammation, and other symptoms commonly experienced by cancer patients.

B. Methods of Forming Therapeutic Compositions

Antibodies can be attached to a therapeutic agent, detectable agent, or nanocarrier using a variety of known cross-linking agents. Methods for covalent or non-covalent attachment of polypeptides are well known in the art. Such methods may include, but are not limited to, use of chemical cross-linkers, photoactivated cross-linkers and/or bifunctional cross-linking reagents. Exemplary methods for cross-linking molecules are disclosed in U.S. Pat. No. 5,603,872 and U.S. Pat. No. 5,401,511. Non-limiting examples of cross-linking reagents include glutaraldehyde, bifunctional oxirane, ethylene glycol diglycidyl ether, carbodiimides such as 1-ethyl-3-(3-dimethylaminopropyl) carbodiimide or dicyclohexylcarbodiimide, bisimidates, dinitrobenzene, N-hydroxysuccinimide ester of suberic acid, disuccinimidyl tartarate, dimethyl-3,3′-dithio-bispropionimidate, azidoglyoxal, N-succinimidyl-3-(2-pyridyldithio)propionate and 4-(bromoadminoethyl)-2-nitrophenylazide.

For antibodies conjugated to nanocarriers (e.g., liposomes), a certain number of antibodies will be present on the surface, i.e., at a given surface density. In some embodiments, the nanocarrier will have at least 5 antibodies per nanocarrier, e.g., at least 10, 30, 40, 50, 75, 100 or higher antibodies per nanocarrier. One of skill in the art will understand that surface density represents an average range, as the number of antibodies per nanocarrier will not be absolutely uniform for all members of the population.

Nanocarriers include vesicles such as liposomes and micelles, as well as polymeric nanoparticles, etc. Nanocarriers are useful for delivery of therapeutic and diagnostic agents, but can be particularly useful for shielding cytotoxic agents used to treat cancer. The nanocarrier can comprise lipids (e.g., phospholipids), hydrophilic polymers, hydrophobic polymers, amphipatic compounds, cross-linked polymers, and a polymeric matrix (see, e.g., WO2009/110939). Depending on the application, the nanocarrier can be designed to have a particular size, half-life, shelf life, and leakage rate.

Preparation of nanocarriers, such as an antibody targeted liposome, polymeric nanoparticle, or extended shelf-life liposome, is described, e.g., in U.S. Pat. Nos. 6,465,188, 7,122,202, 7,462,603 and 7,550,441.

In some embodiments, the antibody is linked to a stabilizing moiety such as PEG, or a liposome or other nanocarrier. U.S. Pat. Nos. 4,732,863 and 7,892,554 and Chattopadhyay et al. (2010) Mol Pharm 7:2194 describe methods for attaching the selected antibody to PEG, PEG derivatives, and nanoparticles (e.g., liposomes). Liposomes containing phosphatidyl-ethanolamine (PE) can be prepared by established procedures as described herein. The inclusion of PE provides an active functional site on the liposomal surface for attachment.

The antibody conjugate can also be formulated to provide more than one active compound, e.g., additional chemotherapeutic or cytotoxic agents, cytokines, or growth inhibitory agents. The active ingredients may also prepared as sustained-release preparations (e.g., semi-permeable matrices of solid hydrophobic polymers (e.g., polyesters, hydrogels (for example, poly(2-hydroxyethyl-methacrylate), or poly(vinylalcohol)), polylactides. The antibodies and immunoconjugates can be entrapped in a nanoparticle prepared, for example, by coacervation techniques or by interfacial polymerization, for example, hydroxymethylcellulose or gelatin microcapsules and poly-(methylmethacylate) microcapsules, respectively, in colloidal drug delivery systems (for example, liposomes, albumin microspheres, microemulsions, nano-particles and nanocapsules) or in macroemulsions.

### VII. Methods of Administration

The anti-receptor IL1RAP antibodies of the invention can efficiently deliver a therapeutic composition to IL1RAP-expressing cells in vivo. In some embodiments, the method of treatment comprises administering to an individual an effective amount of a therapeutic anti-receptor IL1RAP conjugate, e.g., an anti-receptor IL1RAP antibody attached to a therapeutic agent. In some embodiments, the individual has been diagnosed with cancer. In some embodiments, the individual is receiving or has received cancer therapy, e.g., surgery, radiotherapy, or chemotherapy. In some embodiments, the individual has been diagnosed, but the cancer is in remission.

In some embodiments, the anti-receptor IL1RAP conjugate includes a liposome. In some embodiments, the method further comprises monitoring the individual for progression of the cancer. In some embodiments, the dose of the anti-receptor IL1RAP conjugate for each administration is determined based on the therapeutic progress of the individual, e.g., where a higher dose of chemotherapeutic is administered if the individual is not responding sufficiently to therapy.

In some embodiments, the invention can include an antibody or antibody-targeted composition and a physiologically (i.e., pharmaceutically) acceptable carrier. The term “carrier” refers to a typically inert substance used as a diluent or vehicle for a diagnostic or therapeutic agent. The term also encompasses a typically inert substance that imparts cohesive qualities to the composition. Physiologically acceptable carriers can be liquid, e.g., physiological saline, phosphate buffer, normal buffered saline (135-150 mM NaCl), water, buffered water, 0.4% saline, 0.3% glycine, glycoproteins to provide enhanced stability (e.g., albumin, lipoprotein, globulin, etc.), and the like. Since physiologically acceptable carriers are determined in part by the particular composition being administered as well as by the particular method used to administer the composition, there are a wide variety of suitable formulations of pharmaceutical compositions of the present invention (See, e.g., Remington's Pharmaceutical Sciences, 17^{th} ed., 1989).

The compositions of the present invention may be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate. Sugars can also be included for stabilizing the compositions, such as a stabilizer for lyophilized antibody compositions.

Dosage forms can be prepared for mucosal (e.g., nasal, sublingual, vaginal, buccal, or rectal), parenteral (e.g., subcutaneous, intravenous, intramuscular, or intraarterial injection, either bolus or infusion), oral, or transdermal administration to a patient. Examples of dosage forms include, but are not limited to: dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

Injectable (e.g., intravenous) compositions can comprise a solution of the antibody or antibody-targeted composition suspended in an acceptable carrier, such as an aqueous carrier. Any of a variety of aqueous carriers can be used, e.g., water, buffered water, 0.4% saline, 0.9% isotonic saline, 0.3% glycine, 5% dextrose, and the like, and may include glycoproteins for enhanced stability, such as albumin, lipoprotein, globulin, etc. Often, normal buffered saline (135-150 mM NaCl) will be used. The compositions can contain pharmaceutically acceptable auxiliary substances to approximate physiological conditions, such as pH adjusting and buffering agents, tonicity adjusting agents, wetting agents, e.g., sodium acetate, sodium lactate, sodium chloride, potassium chloride, calcium chloride, sorbitan monolaurate, triethanolamine oleate, etc. In some embodiments, the antibody-targeted composition can be formulated in a kit for intravenous administration.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. In the practice of the present invention, compositions can be administered, for example, by intravenous infusion, topically, intraperitoneally, intravesically, or intrathecally. Parenteral administration and intravenous administration are the preferred methods of administration. The formulations of targeted compositions can be presented in unit-dose or multi-dose sealed containers, such as ampoules and vials.

The targeted delivery composition of choice, alone or in combination with other suitable components, can be made into aerosol formulations (“nebulized”) to be administered via inhalation. Aerosol formulations can be placed into pressurized acceptable propellants, such as dichlorodifluoromethane, propane, and nitrogen.

The pharmaceutical preparation can be packaged or prepared in unit dosage form. In such form, the preparation is subdivided into unit doses containing appropriate quantities of the active component, e.g., according to the dose of the therapeutic agent or concentration of antibody. The unit dosage form can be a packaged preparation, the package containing discrete quantities of preparation. The composition can, if desired, also contain other compatible therapeutic agents.

The antibody (or antibody-targeted composition) can be administered by injection or infusion through any suitable route including but not limited to intravenous, subcutaneous, intramuscular or intraperitoneal routes. An example of administration of a pharmaceutical composition includes storing the antibody at 10 mg/ml in sterile isotonic aqueous saline solution for injection at 4° C., and diluting it in either 100 ml or 200 ml 0.9% sodium chloride for injection prior to administration to the patient. The antibody is administered by intravenous infusion over the course of 1 hour at a dose of between 0.2 and 10 mg/kg. In other embodiments, the antibody is administered by intravenous infusion over a period of between 15 minutes and 2 hours. In still other embodiments, the administration procedure is via sub-cutaneous bolus injection.

The dose of antibody is chosen in order to provide effective therapy for the patient and is in the range of less than 0.1 mg/kg body weight to about 25 mg/kg body weight or in the range 1 mg-2 g per patient. In some cases, the dose is in the range 1-100 mg/kg, or approximately 50 mg-8000 mg/patient. The dose may be repeated at an appropriate frequency which may be in the range once per day to once every three months, depending on the pharmacokinetics of the antibody (e.g., half-life of the antibody in the circulation) and the pharmacodynamic response (e.g., the duration of the therapeutic effect of the antibody). In some embodiments, the in vivo half-life of between about 7 and about 25 days and antibody dosing is repeated between once per week and once every 3 months.

Administration can be periodic. Depending on the route of administration, the dose can be administered, e.g., once every 1, 3, 5, 7, 10, 14, 21, or 28 days or longer (e.g., once every 2, 3, 4, or 6 months). In some cases, administration is more frequent, e.g., 2 or 3 times per day. The patient can be monitored to adjust the dosage and frequency of administration depending on therapeutic progress and any adverse side effects, as will be recognized by one of skill in the art.

Thus in some embodiments, additional administration is dependent on patient progress, e.g., the patient is monitored between administrations. For example, after the first administration or round of administrations, the patient can be monitored for rate of tumor growth, recurrence (e.g., in the case of a post-surgical patient), or general disease-related symptoms such as weakness, pain, nausea, etc.

In therapeutic use for the treatment of cancer, an antibody-targeted composition (e.g., including a therapeutic and/or diagnostic agent) can be administered at the initial dosage of about 0.001 mg/kg to about 1000 mg/kg daily and adjusted over time. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosage is varied depending upon the requirements of the patient, the severity of the condition being treated, and the targeted composition being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side-effects that accompany the administration of a particular targeted composition in a particular patient, as will be recognized by the skilled practitioner.

All patents, patent applications, and other publications, including GenBank Accession Numbers, cited in this application are incorporated by reference in the entirety for all purposes.

### VIII. Examples

The following examples are provided by way of illustration only and not by way of limitation. Those of skill in the art will readily recognize a variety of non-critical parameters that could be changed or modified to yield essentially the same or similar results.

### Example I

### Generation of Antibodies that Bind the Receptor Form of IL1RAP

### Design of the Peptide ACPep-1 as an Immunogen for Mouse Immunization

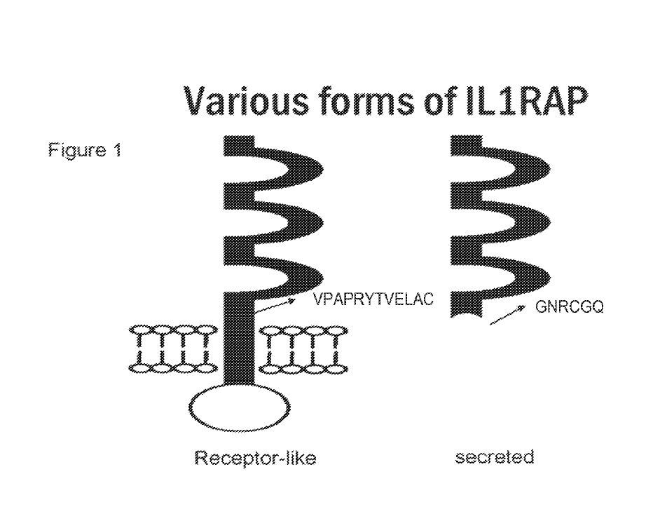

To generate antibody that binds to receptor IL1RAP protein but does not bind to soluble IL1RAP protein, the peptide AC Pep-1 (i.e. VKQKVPAPRYTVELAC, SEQ ID NO.: 47) was designed. This peptide is located proximal to the membrane anchor region (i.e., at residues 347-362), which allows for only recognition of receptor IL1RAP isoforms. (FIG. 1) Monoclonal antibodies raised against this peptide bind the receptor IL1RAP, not soluble IL1RAP.

### Screening of Monoclonal Antibodies to Cell Surface-Associated IL1RAP

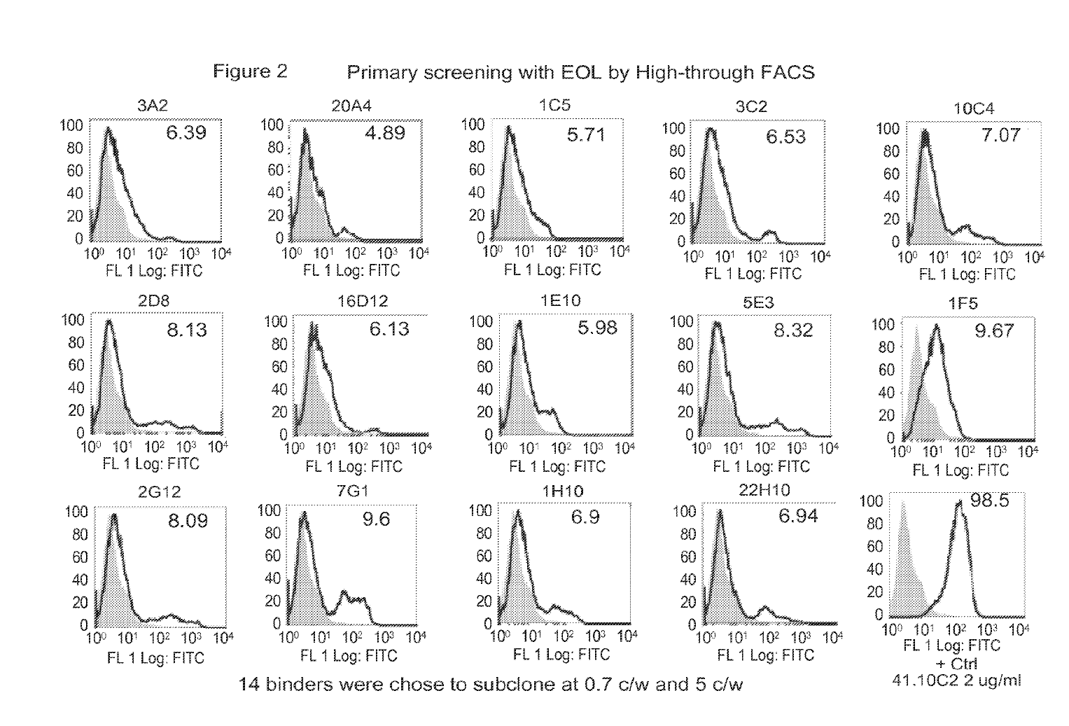

A/J mice were immunized eight times with AC Pep-1 peptide conjugated to keyhole limpet hemocyanin (KLH). Serum antibody titer to receptor IL1RAP on the cell surface was measured by FACS using the IL1RAP expressing cell line, EOL-1. (FIG. 2) Antibody producing cells from the lymph nodes of mice producing anti-IL1RAP antibodies as determined by FACS were fused to create hybridomas.

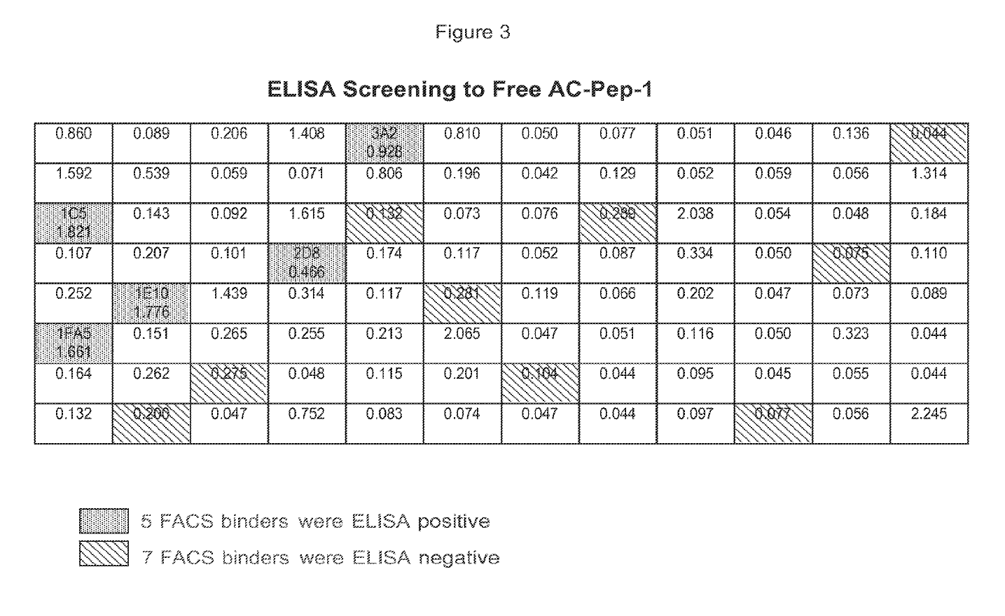

Primary screening of hybridomas was performed by FACS using culture supernatant of hybridoma cells grown in 96-well plates. For supernatants that showed cell-binding activity by FACS, reactivity with the peptide AC Pep-1 was confirmed by ELISA. (FIG. 3) Clones, such as 1F5, that tested positive for binding by FACS and ELISA were subcloned by limited dilution and further characterized.

### Example 11

### Sequence of Anti-IL1RAP Antibody Clone 1F5

The heavy chain and light chain polynucleotides encoding anti-IL1RAP antibody clone 1F5 were determined. The nucleotide sequence was translated into the corresponding amino acid sequence. The deduced amino acid sequences are provided herein.

Light chain variable region protein sequence:

(SEQ ID NO: 5)

DVVMTQTPLSLPVSLGDQASISCRSSQSLVHINGNTYLHWYLQKPGQSPK
LLIYKVSNRFSGVPDRFSGSGSGTDFTLKISRVEAEDLGVYFCSQSTHVP
WTFGGGTKLEIK

Heavy chain variable region protein sequence:

(SEQ ID NO: 6)

EVKLVESGGGLVKPGASLKLSCAASGFTFSNYGMSWVRQTSDKRLEWVAS
ISSGGGSTYYPDNVKGRFTISRENAKNTLYLQMSSLKSEDTALYYCARVL
PGRWAMDYWGQGSSVTVSS

Complementarity determining region (CDR) sequences were determined from the heavy and light chain protein sequences. The deduced amino acid sequences for the CDRs are provided herein.

| CDRH1 | CDRH2 | CDRH3 |
|---|---|---|
| GFTFSNYG | ISSGGGST | ARVLPGRWAMDY |
| (SEQ ID NO: 29) | (SEQ ID NO: 30) | (SEQ ID NO: 31) |
| CDRL1 | CDRL2 | CDRL3 |
| QSLVHINGNTY | KVS | SQSTHVPWT |
| (SEQ ID NO. 32) | (SEQ ID NO: 33) | (SEQ ID NO: 12) |

### Example III

### Characterization of Anti-IL1RAP Antibody Clone 1F5

### 1F5 Binds Specifically to IL1RAP Positive Cell Lines, not Negative Cell Line, JVM-3

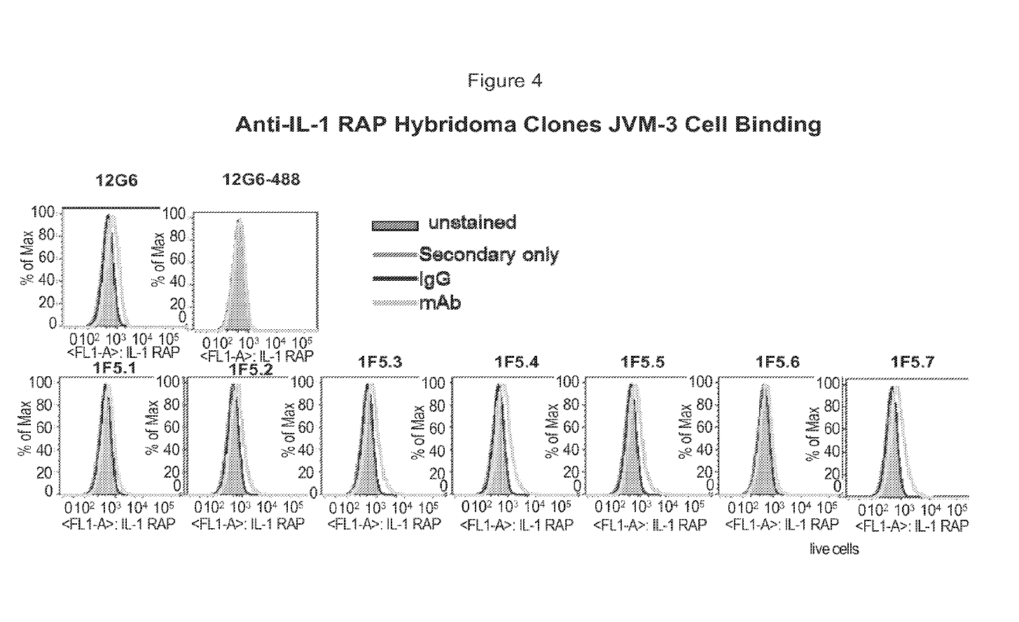

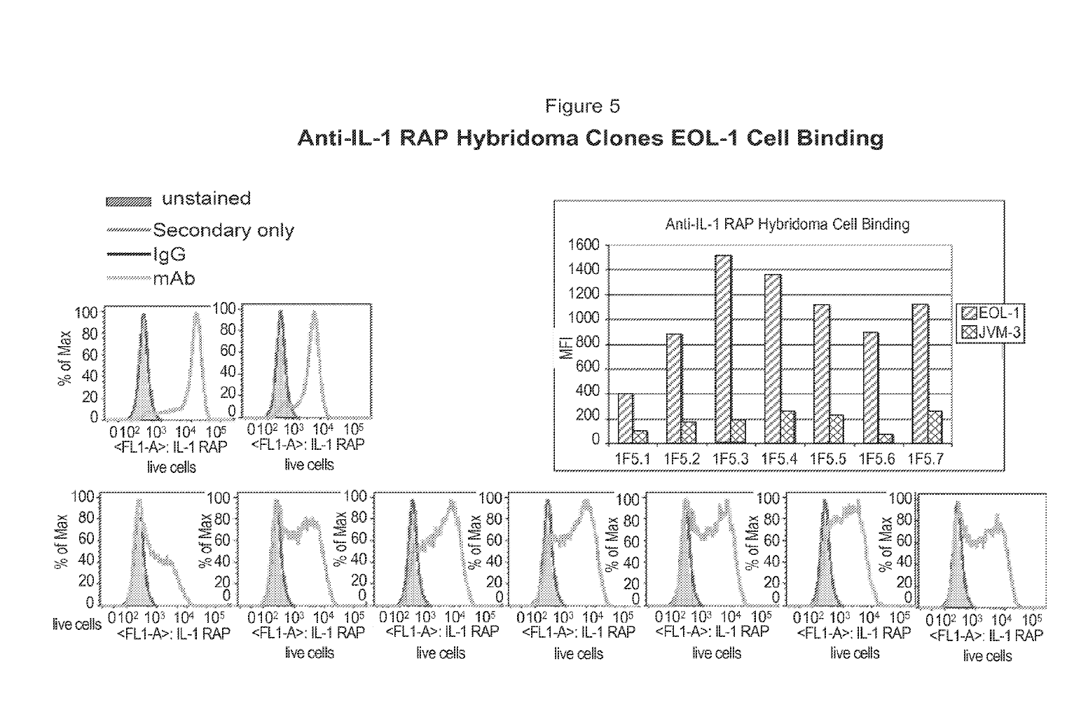

1F5 supernatants were analyzed for binding using EOL and JVM-3 cell lines. All the supernatants tested do not bind JVM-3 cells, an IL1RAP negative cell line (FIG. 4), but do bind EOL-1 cells, an IL1RAP positive cell line, (FIG. 5). This indicates that 1F5 recognizes receptor IL1RAP protein.

1F5 does not Bind Soluble IL1RAP Protein from Normal Human Sera (NHS)

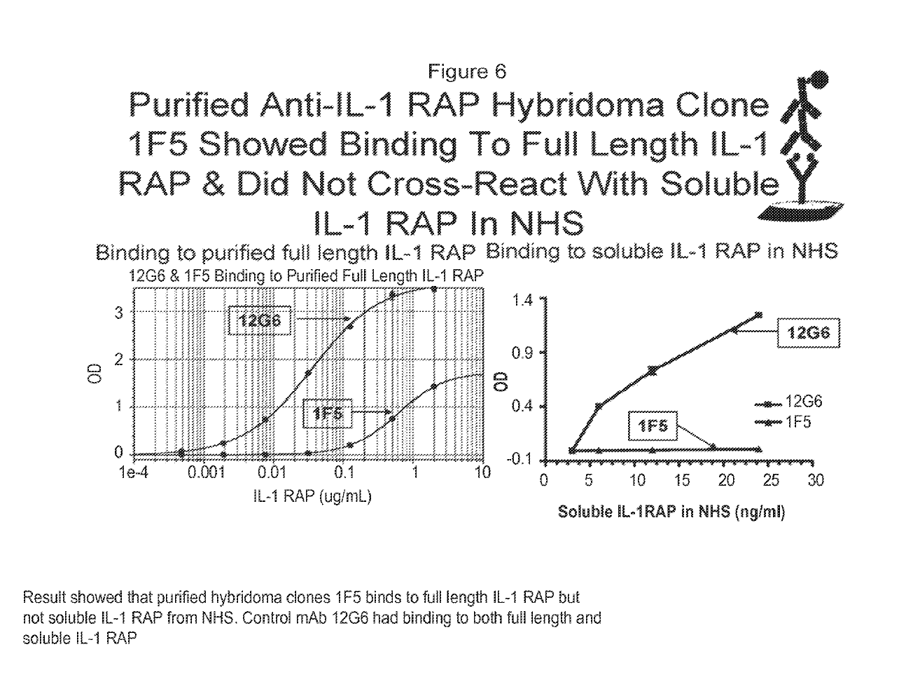

Next, the ability of 1F5 to bind the soluble form of IL1RAP was determined. Sandwich ELISA was performed. Antibody 12G6, which can bind the soluble form of IL1RAP in normal human serum (NHS) was compared as a control. In comparison, 1F5 did not bind IL1RAP from NHS (FIG. 6). This indicates that 1F5 does not bind the soluble form of IL1RAP.

### 1F5 Exhibits Saturation Binding to a Full Length IL1RAP Coated ELISA Plate

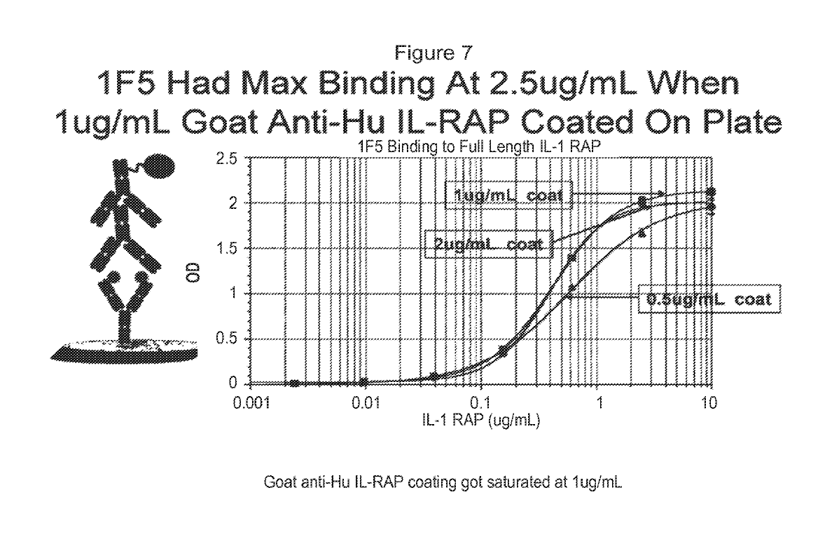

Wells of a 96-well ELISA plate were coated with goat-anti-human IL1RAP polyclonal at 0.5 μg/ml and incubated overnight at 2-8° C. The coated wells were washed with PBS with 0.05% Tween-20, then blocked with 1% BSA. IL1RAP was added into the wells at 0.5, 1, or 2 μg/ml followed by an incubation and a 3×PBS wash. After the 3×PBS wash, antibody 1F5 was added to the wells at 1 μg/ml followed by an incubation and a 3×PBS wash. After the 3×PBS wash, donkey-anti-mouse-HRP was added to the wells at a 1:50,000 dilution followed by an incubation and a 3×PBS wash. Color was developed with TMB substrate. The reaction was quenched with acid and the plate was read at 450 nm. The antibody 1F5 exhibited saturation binding to wells coated with 1 and 2 μg/ml IL1RAP. (FIG. 7) This indicates that 1F5 binds to specific binding sites on IL1RAP.

### 1F5 Binds Specifically to 12-Mer Peptide VPAPRYTVELAC (SEQ ID NO: 3)

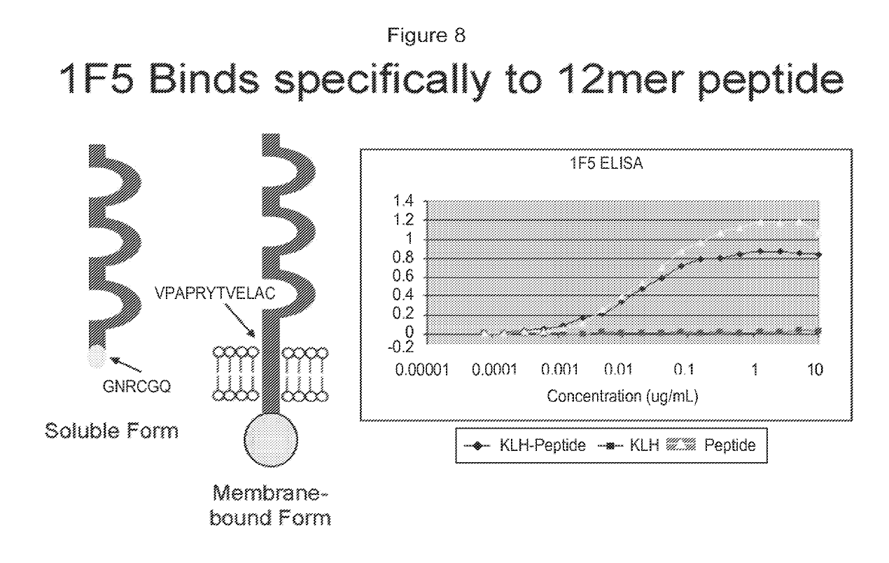

Peptide was conjugated to Keyhole Limpet Hemocyanin (KLH) using methods known in the art. Unconjugated peptide, KLH-peptide, and KLH were then adsorbed onto different wells of a MAXIBIND 96-well ELISA plate, which was then blocked with 1% BSA. Anti-IL1RAP antibody 1F5 was then added to the wells followed by an incubation and a wash. After the wash, donkey-anti-mouse-HRP was added to the wells at a 1:50,000 dilution followed by an incubation and a wash. Color was developed with TMB substrate. The reaction was quenched with acid and the plate was read at 450 nm. Antibody 1F5 exhibited saturation binding to the peptide coated well, and the KLH-peptide coated well, but not the KLH coated well. (FIG. 8) This indicates that 1F5 binds specifically to the VPAPRYTVELAC (SEQ ID NO: 3) peptide, or a portion thereof.

### Expression Analysis by Flow Cytometry

Bone Marrow-Derived Mast Cells (BMMC) or Peripheral Blood Mononuclear Cells (PBMC) were isolated by Ficoll separation (GE Healthcare) from bone marrow aspirate or whole blood respectively of AML patients. Cells were resuspended in staining solution (HBSS, 1% BSA, 1 mM EDTA) to 2×10⁶ cells/mL and blocked with mouse and rat IgG prior to staining. Unconjugated IgG2a or 1F5 antibodies were added to each tube to a final concentration of 2 μg/mL. 50 μL of the cell solution was added to each tube and incubated on ice for 30 min (each stage of incubation was 30 min). Cells were washed with staining solution and resuspended in 100 μL of staining solution with a 2 g/mL concentration of anti-mouse-PE secondary. Cells were washed with staining solution and resuspended in 100 μL of staining solution with 5 μL each of CD34-FITC, CD45-APC and CD38-APC (BD). Cells were washed with staining solution and resuspended in 200 μL. Cells were immediately applied to flow cytometry for analysis.

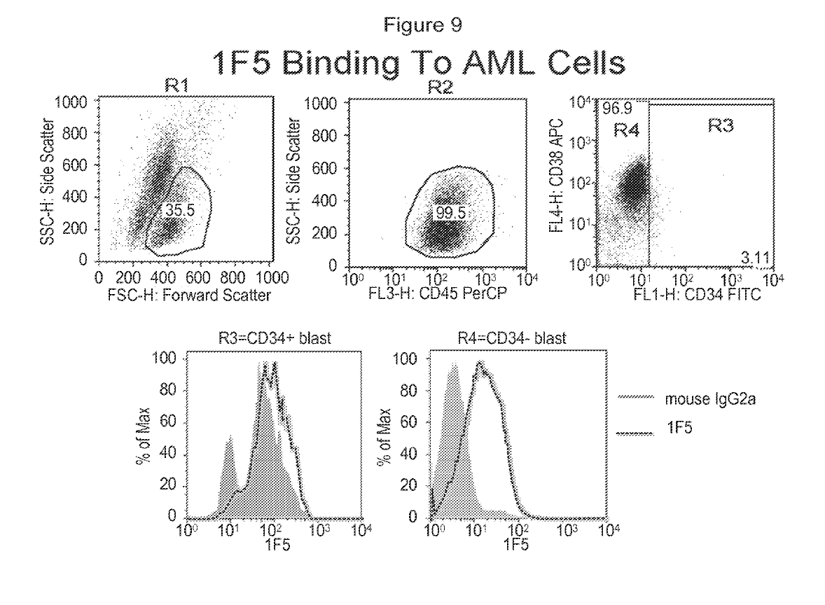

Cells were selected for further analysis on the basis of forward scatter and side scatter (R1) and then side scatter and CD45 expression (R2) as indicated in FIG. 9. Selected cells were then gated based on the presence (R3) and absence (R4) of CD34. Anti-IL1RAP antibody 1F5 showed strong binding to both CD34+ and CD34− cells. (FIG. 9).

### Example IV

### Characterization of Anti-IL1RAP Antibody Clone 1F5

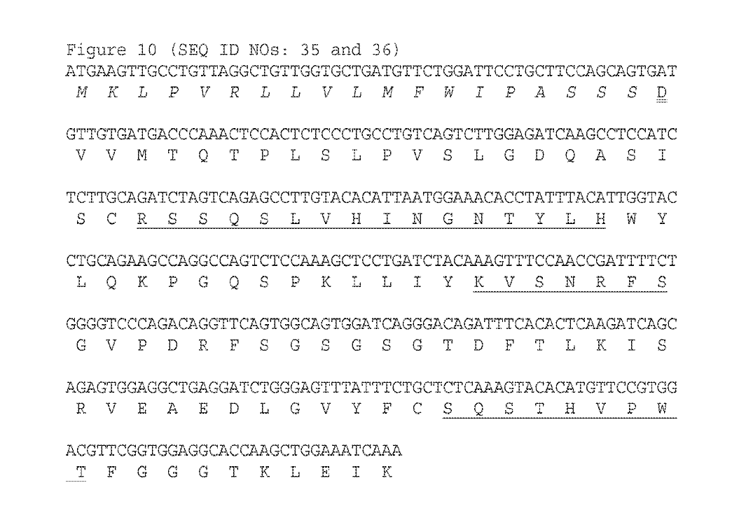

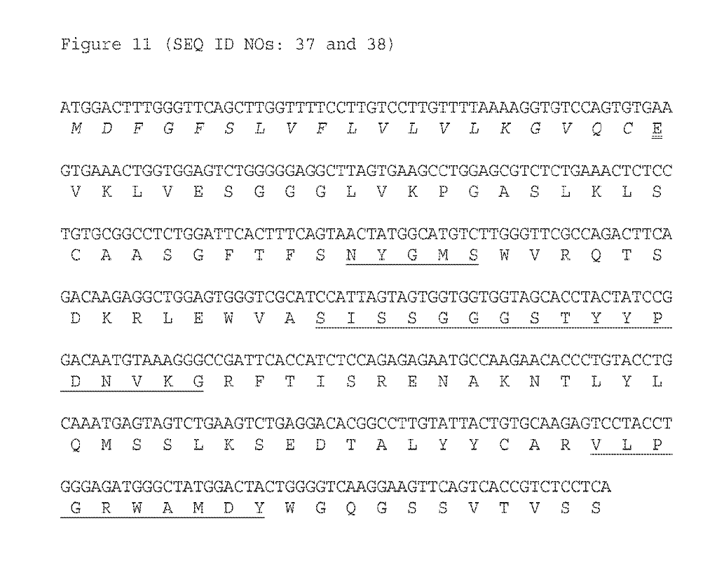

cDNAs sequences coding for 1F5 light and heavy chain variable regions were cloned by standard PCR techniques using degenerate primers from 1F5 hybridoma and their sequences determined. The VL and VH nucleotide sequences and their deduced amino acid sequences are shown in FIGS. 10 and 11 respectively.

### Example V

### Second Screening of Monoclonal Antibodies to Cell Surface-Associated IL1RAP

Three BALB/c mice were immunized against ACPep-1 conjugated to KLH. After mice were boosted seven times with ACPep-1 conjugated to KLH, antibody producing cells from the lymph nodes of mice producing anti-IL1RAP antibodies were fused with mouse myeloma cell line Sp2/0 to generate hybridomas. Monoclonal antibodies from hybridomas were screened by flow cytometry for binding to a Chinese hamster ovary cell transfectant expressing human IL1RAP (CHO-IL1RAP). Three monoclonal antibodies, 4G9, 12F1 and 42E1, recognized CHO-IL1RAP but not the untransfected CHO parental cells. The three hybridomas were expanded and their antibodies were purified by affinity chromatography.

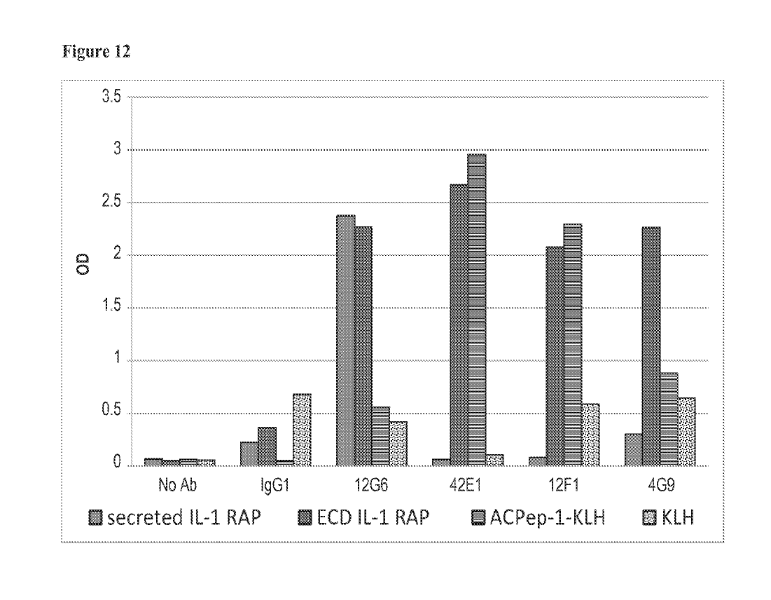

An ELISA was performed to further determine the specificity of monoclonal antibodies 4G9, 12F1, and 42E1. Wells of 96-well plate were coated with the secreted form of IL1RAP, ECD form of IL1RAP, ACPep-1 conjugated to KLH, or unconjugated KLH at 2 ug/ml overnight at 2-8° C. The coated wells were washed with PBS with 0.05% Tween-20, and then blocked with 1% BSA. Purified test antibodies 4G9, 12F1 and 42E1 were diluted to 20 ug/ml, added to wells coated with various proteins, and incubated at RT for 1 hr. Antibody 12G6, which binds to both the ECD and the secreted form of IL1RAP, was added as a control. Mouse IgG1 was also added as an isotype control. After incubation and PBS washes, donkey anti-mouse-HRP polyclonal antibodies were added. Color was developed with TMB substrate after incubation and PBS washes. The reaction was quenched with acid and the plate was read at 450 nm. Results are shown in FIG. 12. Compared to 12G6, 4G9, 12F1 and 42E1 did not recognize the secreted form of IL1RAP, they only recognized the ECD form of IL1RAP and ACPep-1 conjugated KLH. These data confirmed that these three monoclonal antibodies bind to the membrane proximal region of the ECD of IL1RAP, whose sequence is also located in ACPep-1 conjugated KLH.

Two of these monoclonal antibodies, 4G9 and 42E1, were chosen to test if they bind to AML cells from a patient. Bone marrow mononuclear cells from a primary AML patient were stained with 4G9 and 42E1. The CD34+CD38− population, which is enriched for leukemic stem cells, were stained positively with 4G9 and 42E1. These data support the binding specificity of both antibodies for IL1RAP (Table 1).

TABLE 1
Monoclonal antibodies 4G9 and 42E1 bind to leukemic cells
from primary AML patients by flow cytometry analysis.

| Antibody | CD34+ CD38− MFI (% positive) | |
|---|---|---|
| IgG | 3.59 | (9%) |
| 4G9 | 33.1 | (92%) |
| 42E1 | 25.5 | (79%) |

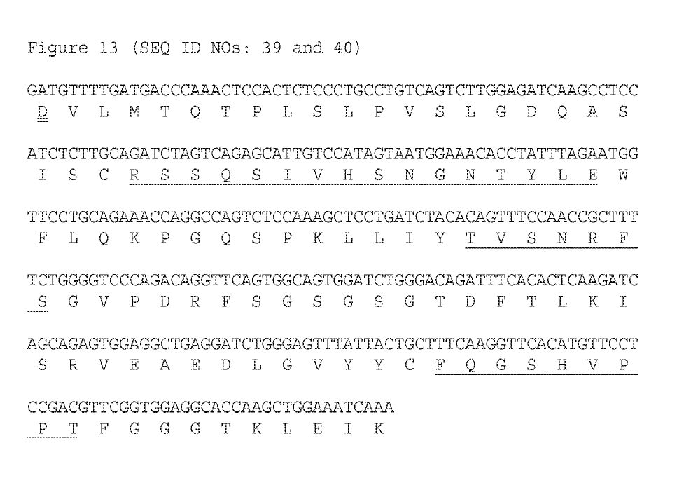

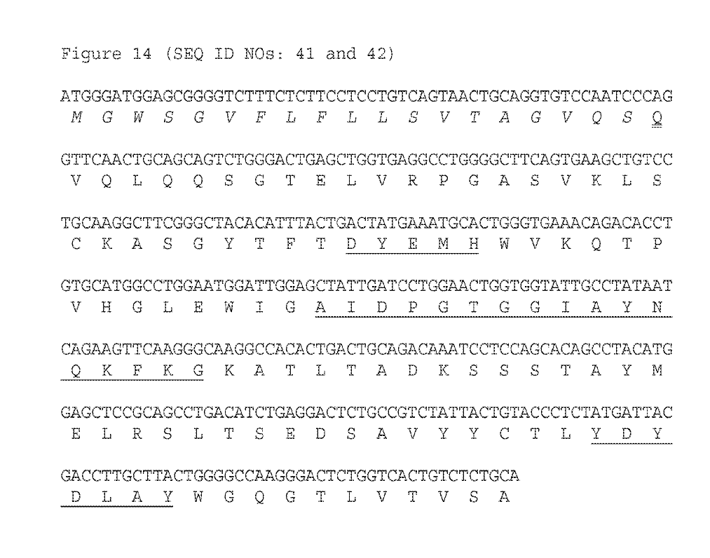

cDNAs coding for 4G9 light and heavy chain variable regions were cloned by standard techniques and their sequences determined. The VL and VH nucleotide sequences and their deduced amino acid sequences are shown in FIGS. 13 and 14 respectively.

### Example VI

### Third Screening of Monoclonal Antibodies to Cell Surface-Associated IL1RAP

Three BALB/c mice were immunized against HNT-34 AML cell lines and boosted alternatively with ACPep-1-KLH and ECD of IL1RAP. After mice were boosted eight times with ACPep-1-KLH and ECD of IL1RAP, antibody producing cells from the lymph nodes of mice producing anti-IL1RAP antibodies were fused with mouse myeloma cell line Sp2/0 to generate hybridomas. Monoclonal antibodies from hybridomas were screened by differentiated ELISA assays for binding to ECD of IL1RAP, but not to the secreted form of IL1RAP. One hybridoma, 4B6, was identified to have such a property. The hybridoma was expanded and its antibody was purified by affinity chromatography.

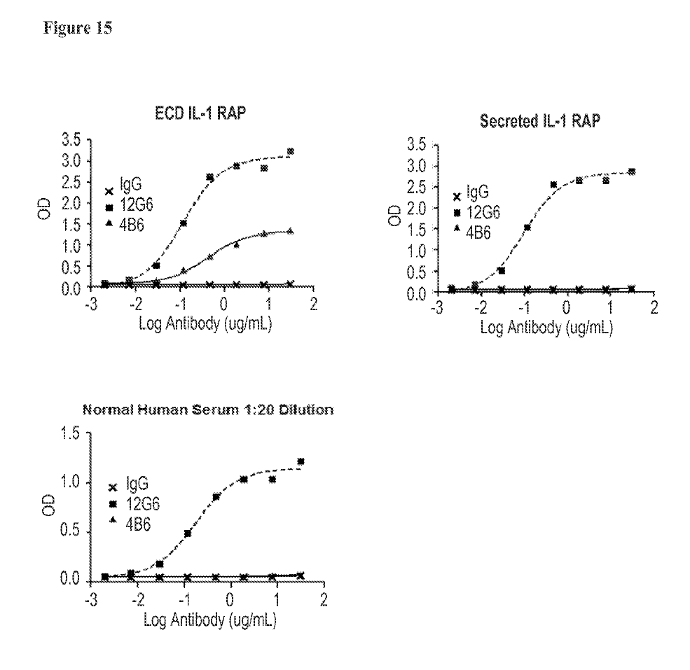

The binding specificity of purified 4B6 was confirmed by ELISA assays described below. Wells of 96-well plate were coated with goat-anti-human IL1RAP polyclonal antibodies to capture various forms of IL1RAP. Forms of IL1RAP captured were (a) ECD form of IL1RAP, (b) the secreted form IL1RAP, and (c) human serum containing the secreted form of IL1RAP. Each captured form was then reacted with serially diluted tested antibody 4B6, control antibody 12G6, or a mouse IgG1 isotype control antibody. After incubation and washing, wells were reacted with secondary donkey-anti-mouse-HRP polyclonal antibodies. Color was developed with TMB substrate after incubation and PBS washes. Results of these ELISA assays shown that, whereas 12G6 reacted to all forms of IL1RAP tested, 4B6 reacted only to the ECD form, it did not bind to the recombinant form of the secreted IL1RAP, nor did it bind to the natural secreted form in the human serum (FIG. 15)

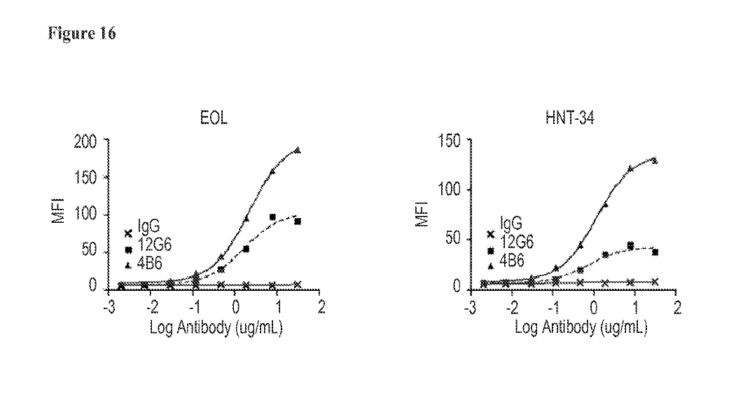

Purified 4B6 antibody was also analyzed for binding to AML cell lines HNT-34 and EOL. The two cell lines were incubated with serially diluted 4B6 and the control antibody 12G6, washed, and then reacted with PE-labeled secondary antibodies for flow cytometry analysis. Data shown in FIG. 16 indicated that 4B6 binds specifically to the two AML cell lines. Other cell lines tested positively for 4B6 by flow cytometry include: HL-60, OCI-AML5, Karpas, and CHO-IL1RAP as indicated in Table 2. Furthermore, 4B6 binds to primary AML patient samples. 4B6 was tested with PBMC from five AML patients, and it stained positively on CD34+ leukemic cells from five AML patients. All these data indicate 4B6 binds specifically to the ECD form of IL1RAP but not the secreted form.

TABLE 2
Summary table for 4B6 binding to IL1RAP expressing cell lines

| Cell | 12G6 | 4B6 |
|---|---|---|
| EOL | ++(>80%) | ++(>80%) |
| HNT | ++(>80%) | ++(>80%) |
| HL-60 | +(>20%) | +++(l00%) |
| OCI-AML5 | ++(>80%) | +(20%) |
| IL-1ARAP CHO | ++(50%) | +(10%) |
| Karpas | +++(100%) | ++80(%) |

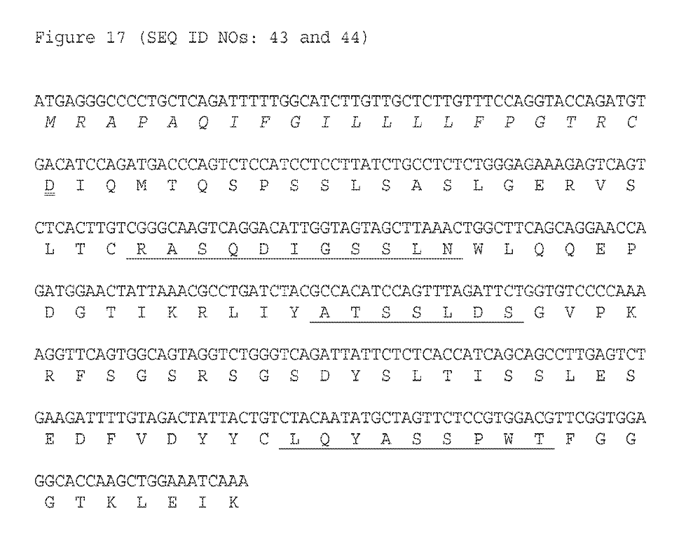

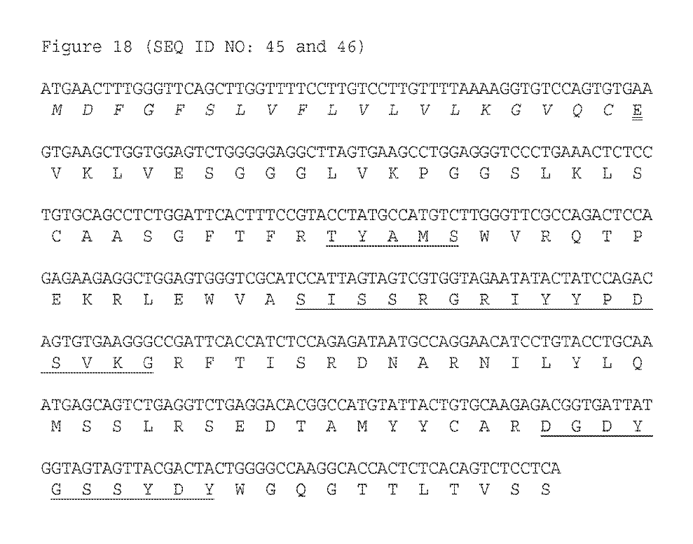

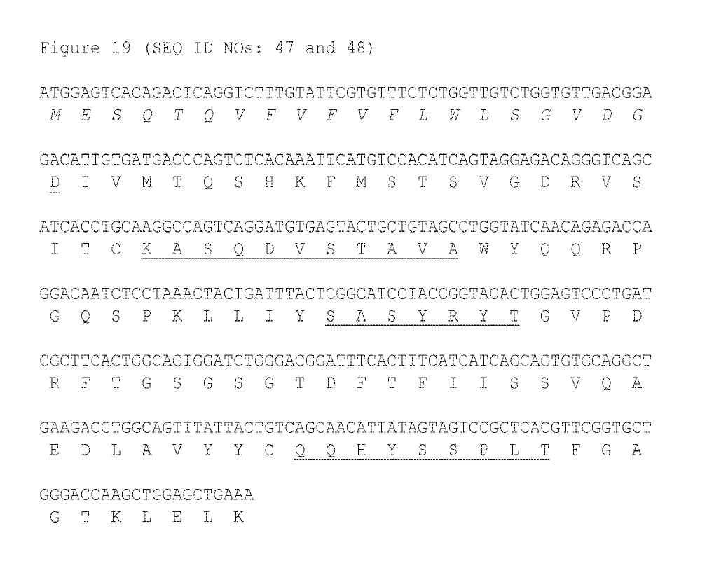

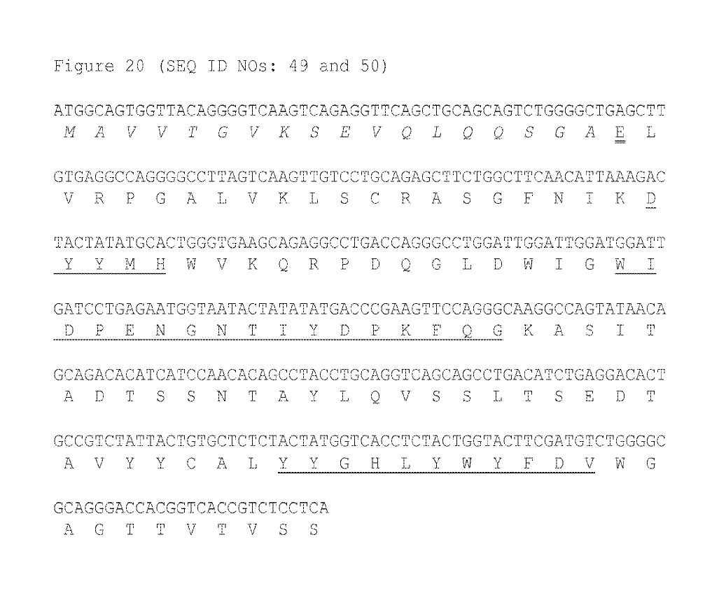

cDNAs coding for 4B6 light and heavy chain variable regions were cloned by standard techniques and their sequences determined. The VL and VH nucleotide sequences and their deduced amino acid sequences are shown in FIGS. 17 and 18, respectively. In some embodiments, the VL and VH nucleotide sequences and their deduced amino acid sequences are those depicted in FIGS. 19 and 20 respectively. In some embodiments, the VL and VH nucleotide sequences are those depicted in FIGS. 17 and 20 respectively. In some embodiments, the VL and VH nucleotide sequences are those depicted in FIGS. 19 and 18 respectively.

It is understood that the examples and embodiments described herein are for illustrative purposes only and that various modifications or changes in light thereof will be suggested to persons skilled in the art and are to be included within the spirit and purview of this application and scope of the appended claims.

What is claimed is:

1. An isolated antibody that binds an extracellular membrane-anchor-proximal region of human IL1RAP.

2. An isolated antibody comprising CDRs of an antibody produced by immunizing an animal against an antigen comprising an extracellular membrane-anchor-proximal region of human IL1RAP.

3. The antibody of claims 1 or 2, wherein the antibody binds an amino acid primary sequence that is within 10 to 15 amino acids of amino acid 361 of human ILR1RAP, wherein human IL1RAP comprises the amino acid sequence set forth in Genebank accession Q9NPH3 (SEQ ID NO: 1).

4. The antibody of claims 1 or 2, wherein the extracellular membrane-anchor-proximal region comprises an amino acid primary sequence VPAPRYTVELAC (SEQ ID NO: 2).

5. The antibody of claim 4, wherein the antibody binds the amino acid primary sequence VPAPRYTVELAC (SEQ ID NO: 2).

6. The antibody of claim 4, wherein the antibody binds the amino acid primary sequence HARSAKGEVAKAAKVKQKVPAPRYTVELACGFGATC (SEQ ID NO: 4).

7. The antibody of claims 1-5, wherein the antibody binds to a membrane-bound form of IL1RAP but does not bind to a soluble form of IL1RAP.

8. The antibody of claims 1-5, wherein the antibody binds to a membrane-bound form of IL1RAP but does not substantially bind to a soluble form of IL1RAP.

9. The antibody of claims 1-5, wherein the antibody binds to a membrane-bound form of IL1RAP with at least 25-fold greater affinity than a soluble form of IL1RAP.

10. The antibody of claims 1-9, wherein the antibody is a humanized antibody.

11. The antibody of claims 1-10, wherein the antibody binds to membrane-bound IL1RAP with a K_{d} of less than about 1, 2, 3, 4, 5, 6, 7, 10, 15, 20, 25, 30, 40, or 50 nM.

12. The antibody of claims 1-11, wherein the antibody inhibits cancer stem cell growth, metabolic activity, viability, or division.

13. The antibody of claims 1-12, wherein the antibody light chain comprises an amino acid sequence that is at least 85%, 90%, 95%, or 100% identical to SEQ ID NO: 5 or the antibody heavy chain comprises an amino acid sequence that is at least 85%, 90%, 95%, or 100% identical to SEQ ID NO: 6.

14. The antibody of claim 13, wherein the light chain sequence is SEQ ID NO: 5 and the heavy chain sequence is SEQ ID NO: 6.

15. The antibody of claims 1-12, wherein the antibody comprises heavy chain complementarity determining regions of antibody clone 1F5, or light chain complementarity determining regions of antibody clone 1F5.

16. The antibody of claims 1-12, wherein one, two, three, four, five, or six antibody complementarity determining regions are selected from the group consisting of SEQ ID NO: 7, SEQ ID NO: 8, SEQ ID NO: 9, SEQ ID NO: 10, SEQ ID NO: 11, and SEQ ID NO: 12.

17. The antibody of claims 1-12, wherein the antibody comprises
a CDRH1 identical to SEQ ID NO: 7;
a CDRH2 identical to SEQ ID NO: 8:
a CDRH3 identical to SEQ ID NO: 9;
a CDRL1 identical to SEQ ID NO: 10;
a CDRL2 identical to SEQ ID NO: 11; and
a CDRL3 identical to SEQ ID NO: 12.

18. The antibody of claims 1-12, wherein the antibody light chain variable region comprises an amino acid sequence that is at least 85%, 90%, 95%, or 100% identical to the antibody light chain variable region of antibody clone 42E1, or the antibody heavy chain variable region comprises an amino acid sequence that is at least 85%, 90%, 95%, or 100% identical to the antibody heavy chain variable region of antibody clone 42E1.

19. The antibody of claims 1-12, wherein the antibody comprises heavy chain and light chain complementarity determining regions of antibody clone 42E1.

20. The antibody of claims 1-12, wherein the antibody comprises one, two, three, four, five, or six antibody complementarity determining regions of antibody clone 42E1.

21. The antibody of claims 1-12, wherein the antibody comprises
a CDRH1 identical to a CDRH1 of antibody clone 42E1;
a CDRH2 identical to a CDRH2 of antibody clone 42E1;
a CDRH3 identical to a CDRH3 of antibody clone 42E1;
a CDRL1 identical to a CDRL1 of antibody clone 42E1;
a CDRL2 identical to a CDRL2 of antibody clone 42E1; and
a CDRL3 identical to a CDRL3 of antibody clone 42E1.

22. The antibody of claims 1-12, wherein the antibody light chain variable region comprises an amino acid sequence that is at least 85%, 90%, 95%, or 100% identical to the antibody light chain variable region of antibody clone 12F1, or the antibody heavy chain variable region comprises an amino acid sequence that is at least 85%, 90%, 95%, or 100% identical to the antibody heavy chain variable region of antibody clone 12F1.

23. The antibody of claims 1-12, wherein the antibody comprises heavy chain and light chain complementarity determining regions of antibody clone 12F1.

24. The antibody of claims 1-12, wherein the antibody comprises one, two, three, four, five, or six antibody complementarity determining regions of antibody clone 12F1.

25. The antibody of claims 1-12, wherein the antibody comprises
a CDRH1 identical to a CDRH1 of antibody clone 12F1;
a CDRH2 identical to a CDRH2 of antibody clone 12F1;
a CDRH3 identical to a CDRH3 of antibody clone 12F1;
a CDRL11 identical to a CDRL11 of antibody clone 12F1;
a CDRL2 identical to a CDRL2 of antibody clone 12F1; and
a CDRL3 identical to a CDRL3 of antibody clone 12F1

26. The antibody of claims 1-12, wherein the antibody light chain variable region comprises an amino acid sequence that is at least 85%, 90%, 95%, or 100% identical to SEQ ID NO: 13 or the antibody heavy chain variable region comprises an amino acid sequence that is at least 85%, 90%, 95%, or 100% identical to SEQ ID NO: 14.

27. The antibody of claim 26, wherein the light chain variable region sequence is SEQ ID NO: 13 and the heavy chain variable region sequence is SEQ ID NO: 14.

28. The antibody of claims 1-12, wherein the antibody comprises heavy chain complementarity determining regions of antibody clone 4G9, or light chain complementarity determining regions of antibody clone 4G9.

29. The antibody of claims 1-12, wherein one, two, three, four, five, or six antibody complementarity determining regions are selected from the group consisting of SEQ ID NO: 15, SEQ ID NO: 16, SEQ ID NO: 17, SEQ ID NO: 18, SEQ ID NO: 19, and SEQ ID NO: 20.

30. The antibody of claims 1-12, wherein the antibody comprises
a CDRH1 identical to SEQ ID NO: 15;
a CDRH2 identical to SEQ ID NO: 16;
a CDRH3 identical to SEQ ID NO: 17;
a CDRL11 identical to SEQ ID NO: 18;
a CDRL2 identical to SEQ ID NO: 19; and
a CDRL3 identical to SEQ ID NO: 20.

31. The antibody of claims 1-12, wherein the antibody light chain variable region comprises an amino acid sequence that is at least 85%, 90%, 95%, or 100% identical to SEQ ID NO: 21 or the antibody heavy chain variable region comprises an amino acid sequence that is at least 85%, 90%, 95%, or 100% identical to SEQ ID NO: 22.

32. The antibody of claim 31, wherein the light chain variable region sequence is SEQ ID NO: 21 and the heavy chain variable region sequence is SEQ ID NO: 22.

33. The antibody of claims 1-12, wherein the antibody comprises heavy chain complementarity determining regions of antibody clone 4B6, or light chain complementarity determining regions of antibody clone 4B6.

34. The antibody of claims 1-12, wherein one, two, three, four, five, or six antibody complementarity determining regions are selected from the group consisting of SEQ ID NO: 23, SEQ ID NO: 24, SEQ ID NO: 25, SEQ ID NO: 26, SEQ ID NO: 27, and SEQ ID NO: 28.

35. The antibody of claims 1-12, wherein the antibody comprises
a CDRH1 identical to SEQ ID NO: 23;
a CDRH2 identical to SEQ ID NO: 24;
a CDRH3 identical to SEQ ID NO: 25;
a CDRL1 identical to SEQ ID NO: 26;
a CDRL2 identical to SEQ ID NO: 27; and
a CDRL3 identical to SEQ ID NO: 28.

36. The antibody of claims 1-12, wherein the antibody light chain variable region comprises an amino acid sequence that is at least 85%, 90%, 95%, or 100% identical to at least 90 consecutive amino acids of SEQ ID NO: 48, or the antibody heavy chain variable region comprises an amino acid sequence that is at least 85%, ⁹⁰%, 95%, or 100% identical to at least 90 consecutive amino acids of SEQ ID NO: 50.

37. The antibody of claims 1-12, wherein the antibody light chain variable region comprises an amino acid sequence that is at least 85%, 90%, 95%, or 100% identical to at least 90 consecutive amino acids of SEQ ID NO: 44, or the antibody heavy chain variable region comprises an amino acid sequence that is at least 85%, 90%, 95%, or 100% identical to at least 90 consecutive amino acids of SEQ ID NO: 46

38. The antibody of claims 1-12, wherein the antibody light chain variable region comprises an amino acid sequence that is at least 85%, 90%, 95%, or 100% identical to at least 90 consecutive amino acids of SEQ ID NO: 48, or the antibody heavy chain variable region comprises an amino acid sequence that is at least 85%, 90%, 95%, or 100% identical to at least 90 consecutive amino acids of SEQ ID NO: 46.

39. The antibody of claims 1-12, wherein the antibody light chain variable region comprises an amino acid sequence that is at least 85%, 90%, 95%, or 100% identical to at least 90 consecutive amino acids of SEQ ID NO: 44, or the antibody heavy chain variable region comprises an amino acid sequence that is at least 85%, 90%, 95%, or 100% identical to at least 90 consecutive amino acids of SEQ ID NO: 50.

40. The antibody of claim 36, wherein the light chain variable region sequence is 100% identical to at least 90 consecutive amino acids of SEQ ID NO: 48, and the heavy chain variable region sequence is 100% identical to at least 90 consecutive amino acids of SEQ ID NO: 50.

41. The antibody of claim 37, wherein the light chain variable region sequence is 100% identical to at least 90 consecutive amino acids of SEQ ID NO: 44, and the heavy chain variable region sequence is 100% identical to at least 90 consecutive amino acids of SEQ ID NO: 46.

42. The antibody of claim 38, wherein the light chain variable region sequence is 100% identical to at least 90 consecutive amino acids of SEQ ID NO: 48, and the heavy chain variable region sequence is 100% identical to at least 90 consecutive amino acids of SEQ ID NO: 46.

43. The antibody of claim 39, wherein the light chain variable region sequence is 100% identical to at least 90 consecutive amino acids of SEQ ID NO: 44, and the heavy chain variable region sequence is 100% identical to at least 90 consecutive amino acids of SEQ ID NO: 50.

44. The antibody of claims 1-12, wherein one, two, three, four, five, or six antibody complementarity determining regions are selected from the group consisting of SEQ ID NO: 51, SEQ ID NO: 52, SEQ ID NO: 53, SEQ ID NO: 54, SEQ ID NO: 55, and SEQ ID NO: 56.

45. The antibody of claims 1-12, wherein one, two, three, four, five, or six antibody complementarity determining regions are selected from the group consisting of SEQ ID NO: 51, SEQ ID NO: 52, SEQ ID NO: 53, SEQ ID NO: 54, SEQ ID NO: 55, SEQ ID NO: 56, SEQ ID NO: 23, SEQ ID NO: 24, SEQ ID NO: 25, SEQ ID NO: 26, SEQ ID NO: 27, and SEQ ID NO: 28.

46. The antibody of claims 1-12, wherein the antibody comprises
a CDRH1 identical to SEQ ID NO: 51;
a CDRH2 identical to SEQ ID NO: 52;
a CDRH3 identical to SEQ ID NO: 53;
a CDRL1 identical to SEQ ID NO: 54;
a CDRL2 identical to SEQ ID NO: 55; and
a CDRL3 identical to SEQ ID NO: 56.

47. The antibody of claims 1-12, wherein the antibody comprises
a CDRH1 identical to SEQ ID NO: 51;
a CDRH2 identical to SEQ ID NO: 52;
a CDRH3 identical to SEQ ID NO: 53;
a CDRL11 identical to SEQ ID NO: 26;
a CDRL2 identical to SEQ ID NO: 27; and
a CDRL3 identical to SEQ ID NO: 28.

48. The antibody of claims 1-12, wherein the antibody comprises
a CDRH1 identical to SEQ ID NO: 23;
a CDRH2 identical to SEQ ID NO: 24;
a CDRH3 identical to SEQ ID NO: 25;
a CDRL1 identical to SEQ ID NO: 54;
a CDRL2 identical to SEQ ID NO: 55; and
a CDRL3 identical to SEQ ID NO: 56.

49. An antibody comprising CDRs of an antibody that binds to a membrane-bound form of IL1RAP but does not substantially bind to a soluble form of IL1RAP.

50. The antibody of claim 51, wherein the antibody is an Fab, F(ab′)₂, an Fab′, or a single chain Fv.

51. The antibody of claims 1-52, wherein the antibody is conjugated to a detectable label selected from the group consisting of a fluorophore, a radionuclide, a phosphorescent agent, a chemiluminescent agent, an enzyme, and an MRI contrast agent.

52. The antibody of claims 1-52, wherein the antibody is conjugated to a chemotherapeutic or a cytotoxic agent selected from the group consisting of an alkylating agent, an antimetabolites, an antitumor antibiotic, hydroxyurea, a platinum-based chemotherapeutic agent, a taxane, bortezomib, lenalidomide, and thalidomide.

53. The antibody of claims 1-52, wherein the antibody binds to CD45+ cells.

54. The antibody of claims 1-52, wherein the antibody binds to cancer cells, myelodysplastic syndrome (MDS) cells, acute myeloid leukemia (AML) or chronic myelogenous leukemia (CML) cells, anaplastic large-cell lymphoma (ALCL) cells, Hodgkins Lymphoma cells, or Non Hodgkins Lymphoma cells such as EOL-1 cells or Karpas 299 cells.

55. The antibody of claim 56, wherein the antibody specifically binds the receptor form of IL1RAP and at least 80% of cells in a culture of EOL-1 cells.

56. The antibody of claims 1-52, wherein the antibody binds to CD45+CD38+ cells.

57. The antibody of claim 58, wherein the antibody binds to CD34+ or CD34− cells.

58. The antibody of claim 56, wherein the antibody binds AML, CML, ALCL, Hodgkins Lymphoma, Non Hodgkins Lymphoma, or MDS stem cells.

59. A formulation comprising a pharmaceutically acceptable excipient, and the antibody of claims 1-60.

60. A method of treating a subject suffering from a disease or condition comprising administering an effective amount of the antibody of claims 1-60, or the formulation of claim 61.

61. The method of claim 62, wherein the disease or condition is cancer or MDS.

62. The method of claim 63, wherein the cancer is AML, CML, ALCL, Hodgkins Lymphoma, or Non Hodgkins Lymphoma.

63. The method of claim 62, wherein the disease or condition is a joint, bone, or muscle disease; a hereditary systemic auto-inflammatory disease; a systemic inflammatory disease; or a common inflammatory disease.


SEQUENCE LISTING (47 sequences)

SEQ ID NO: 1 - protein, 6 aa, Homo sapiens
GNRCGQ

SEQ ID NO: 2 - protein, 570 aa, Homo sapiens
MTLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVELACGFGATVLLVVILIVVYHVYWLEMVLFYRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNV

SEQ ID NO: 3 - protein, 12 aa, Homo sapiens
VPAPRYTVELAC

SEQ ID NO: 4 - protein, 36 aa, Homo sapiens
HARSAKGEVAKAAKVKQKVPAPRYTVELACGFGATC

SEQ ID NO: 5 - protein, 112 aa, Artificial Sequence
  synthetic anti-receptor IL1RAP antibody light chain
DVVMTQTPLSLPVSLGDQASISCRSSQSLVHINGNTYLHWYLQKPGQSPKLLIYKVSNRFSGVPDRFSGSGSGTDFTLKISRVEAEDLGVYFCSQSTHVPWTFGGGTKLEIK

SEQ ID NO: 6 - protein, 119 aa, Artificial Sequence
  synthetic anti-receptor IL1RAP antibody heavy chain
EVKLVESGGGLVKPGASLKLSCAASGFTFSNYGMSWVRQTSDKRLEWVASISSGGGSTYYPDNVKGRFTISRENAKNTLYLQMSSLKSEDTALYYCARVLPGRWAMDYWGQGSSVTVSS

SEQ ID NO: 7 - protein, 5 aa, Artificial Sequence
  synthetic anti-receptor IL1RAP antibody 1F5 CDRH1
NYGMS

SEQ ID NO: 8 - protein, 17 aa, Artificial Sequence
  synthetic anti-receptor IL1RAP antibody 1F5 CDRH2
SISSGGGSTYYPDNVKG

SEQ ID NO: 9 - protein, 10 aa, Artificial Sequence
  synthetic anti-receptor IL1RAP antibody 1F5 CDRH3
VLPGRWAMDY

SEQ ID NO: 10 - protein, 16 aa, Artificial Sequence
  synthetic anti-receptor IL1RAP antibody 1F5 CDRL1
RSSQSLVHINGNTYLH

SEQ ID NO: 11 - protein, 7 aa, Artificial Sequence
  synthetic anti-receptor IL1RAP antibody 1F5 CDRL2
KVSNRFS

SEQ ID NO: 12 - protein, 9 aa, Artificial Sequence
  synthetic anti-receptor IL1RAP antibody 1F5 CDRL3
SQSTHVPWT

SEQ ID NO: 13 - protein, 112 aa, Artificial Sequence
  synthetic anti-receptor IL1RAP antibody light chain
DVLMTQTPLSLPVSLGDQASISCRSSQSIVHSNGNTYLEWFLQKPGQSPKLLIYTVSNRFSGVPDRFSGSGSGTDFTLKISRVEAEDLGVYYCFQGSHVPPTFGGGTKLEIK

SEQ ID NO: 14 - protein, 116 aa, Artificial Sequence
  synthetic anti-receptor IL1RAP antibody heavy chain
QVQLQQSGTELVRPGASVKLSCKASGYTFTDYEMHWVKQTPVHGLEWIGAIDPGTGGIAYNQKFKGKATLTADKSSSTAYMELRSLTSEDSAVYYCTLYDYDLAYWGQGTLVTVSA

SEQ ID NO: 15 - protein, 5 aa, Artificial Sequence
  synthetic anti-receptor IL1RAP antibody 4G9 CDRH1
DYEMH

SEQ ID NO: 16 - protein, 17 aa, Artificial Sequence
  synthetic anti-receptor IL1RAP antibody 4G9 CDRH2
AIDPGTGGIAYNQKFKG

SEQ ID NO: 17 - protein, 7 aa, Artificial Sequence
  synthetic anti-receptor IL1RAP antibody 4G9 CDRH3
YDYDLAY

SEQ ID NO: 18 - protein, 16 aa, Artificial Sequence
  synthetic anti-receptor IL1RAP antibody 4G9 CDRL1
RSSQSIVHSNGNTYLE

SEQ ID NO: 19 - protein, 7 aa, Artificial Sequence
  synthetic anti-receptor IL1RAP antibody 4G9 CDRL2
TVSNRFS

SEQ ID NO: 20 - protein, 9 aa, Artificial Sequence
  synthetic anti-receptor IL1RAP antibody 4G9 CDRL3
FQGSHVPPT

SEQ ID NO: 21 - protein, 107 aa, Artificial Sequence
  synthetic anti-receptor IL1RAP antibody light chain
DIQMTQSPSSLSASLGERVSLTCRASQDIGSSLNWLQQEPDGTIKRLIYATSSLDSGVPKRFSGSRSGSDYSLTISSLESEDFVDYYCLQYASSPWTFGGGTKLEIK

SEQ ID NO: 22 - protein, 118 aa, Artificial Sequence
  synthetic anti-receptor IL1RAP antibody heavy chain
EVKLVESGGGLVKPGGSLKLSCAASGFTFRTYAMSWVRQTPEKRLEWVASISSRGRIYYPDSVKGRFTISRDNARNILYLQMSSLRSEDTAMYYCARDGDYGSSYDYWGQGTTLTVSS

SEQ ID NO: 23 - protein, 5 aa, Artificial Sequence
  synthetic anti-receptor IL1RAP antibody 4B6 CDRH1
TYAMS

SEQ ID NO: 24 - protein, 16 aa, Artificial Sequence
  synthetic anti-receptor IL1RAP antibody 4B6 CDRH2
SISSRGRIYYPDSVKG

SEQ ID NO: 25 - protein, 10 aa, Artificial Sequence
  synthetic anti-receptor IL1RAP antibody 4B6 CDRH3
DGDYGSSYDY

SEQ ID NO: 26 - protein, 11 aa, Artificial Sequence
  synthetic anti-receptor IL1RAP antibody 4B6 CDRL1
RASQDIGSSLN

SEQ ID NO: 27 - protein, 7 aa, Artificial Sequence
  synthetic anti-receptor IL1RAP antibody 4B6 CDRL2
ATSSLDS

SEQ ID NO: 28 - protein, 9 aa, Artificial Sequence
  synthetic anti-receptor IL1RAP antibody 4B6 CDRL3
LQYASSPWT

SEQ ID NO: 29 - protein, 8 aa, Artificial Sequence
  synthetic anti-receptor IL1RAP antibody 1F5 CDRH1
GFTFSNYG

SEQ ID NO: 30 - protein, 8 aa, Artificial Sequence
  synthetic anti-receptor IL1RAP antibody 1F5 CDRH2
ISSGGGST

SEQ ID NO: 31 - protein, 12 aa, Artificial Sequence
  synthetic anti-receptor IL1RAP antibody 1F5 CDRH3
ARVLPGRWAMDY

SEQ ID NO: 32 - protein, 11 aa, Artificial Sequence
  synthetic anti-receptor IL1RAP antibody 1F5 CDRL1
QSLVHINGNTY

SEQ ID NO: 33 - protein, 3 aa, Artificial Sequence
  synthetic anti-receptor IL1RAP antibody 1F5 CDRL2
KVS

SEQ ID NO: 34 - protein, 356 aa, Homo sapiens
MTLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKGNRCGQ

SEQ ID NO: 35 - DNA, 393 bp, Artificial Sequence
  Synthetic cDNA for anti-receptor IL1RAP antibody IF5 VL chain
atgaagttgcctgttaggctgttggtgctgatgttctggattcctgcttccagcagtgatgttgtgatgacccaaactccactctccctgcctgtcagtcttggagatcaagcctccatctcttgcagatctagtcagagccttgtacacattaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 36 - protein, 131 aa, Artificial Sequence
  Synthetic anti-receptor IL1RAP antibody 1F5 VL chain
MKLPVRLLVLMFWIPASSSDVVMTQTPLSLPVSLGDQASISCRSSQSLVHINGNTYLHWYLQKPGQSPKLLIYKVSNRFSGVPDRFSGSGSGTDFTLKISRVEAEDLGVYFCSQSTHVPWTFGGGTKLEIK

SEQ ID NO: 37 - DNA, 414 bp, Artificial Sequence
  Synthetic cDNA for anti-receptor IL1RAP antibody 1F5 VH chain
atggactttgggttcagcttggttttccttgtccttgttttaaaaggtgtccagtgtgaagtgaaactggtggagtctgggggaggcttagtgaagcctggagcgtctctgaaactctcctgtgcggcctctggattcactttcagtaactatggcatgtcttgggttcgccagacttcagacaagaggctggagtgggtcgcatccattagtagtggtggtggtagcacctactatccggacaatgtaaagggccgattcaccatctccagagagaatgccaagaacaccctgtacctgcaaatgagtagtctgaagtctgaggacacggccttgtattactgtgcaagagtcctacctgggagatgggctatggactactggggtcaaggaagttcagtcaccgtctcctca

SEQ ID NO: 38 - protein, 138 aa, Artificial Sequence
  Synthetic anti-receptor IL1RAP antibody 1F5 VH chain
MDFGFSLVFLVLVLKGVQCEVKLVESGGGLVKPGASLKLSCAASGFTFSNYGMSWVRQTSDKRLEWVASISSGGGSTYYPDNVKGRFTISRENAKNTLYLQMSSLKSEDTALYYCARVLPGRWAMDYWGQGSSVTVSS

SEQ ID NO: 39 - DNA, 336 bp, Artificial Sequence
  Synthetic cDNA for anti-receptor IL1RAP antibody 4G9 VL chain
gatgttttgatgacccaaactccactctccctgcctgtcagtcttggagatcaagcctccatctcttgcagatctagtcagagcattgtccatagtaatggaaacacctatttagaatggttcctgcagaaaccaggccagtctccaaagctcctgatctacacagtttccaaccgcttttctggggtcccagacaggttcagtggcagtggatctgggacagatttcacactcaagatcagcagagtggaggctgaggatctgggagtttattactgctttcaaggttcacatgttcctccgacgttcggtggaggcaccaagctggaaatcaaa

SEQ ID NO: 40 - protein, 112 aa, Artificial Sequence; identical to SEQ ID NO: 13
  Synthetic anti-receptor IL1RAP antibody 4G9 VL chain

SEQ ID NO: 41 - DNA, 405 bp, Artificial Sequence
  Synthetic cDNA for anti-receptor IL1RAP antibody 4G9 VH chain
atgggatggagcggggtctttctcttcctcctgtcagtaactgcaggtgtccaatcccaggttcaactgcagcagtctgggactgagctggtgaggcctggggcttcagtgaagctgtcctgcaaggcttcgggctacacatttactgactatgaaatgcactgggtgaaacagacacctgtgcatggcctggaatggattggagctattgatcctggaactggtggtattgcctataatcagaagttcaagggcaaggccacactgactgcagacaaatcctccagcacagcctacatggagctccgcagcctgacatctgaggactctgccgtctattactgtaccctctatgattacgaccttgcttactggggccaagggactctggtcactgtctctgca

SEQ ID NO: 42 - protein, 135 aa, Artificial Sequence
  Synthetic anti-receptor IL1RAP antibody 4G9 VH chain
MGWSGVFLFLLSVTAGVQSQVQLQQSGTELVRPGASVKLSCKASGYTFTDYEMHWVKQTPVHGLEWIGAIDPGTGGIAYNQKFKGKATLTADKSSSTAYMELRSLTSEDSAVYYCTLYDYDLAYWGQGTLVTVSA

SEQ ID NO: 43 - DNA, 381 bp, Artificial Sequence
  Synthetic cDNA for anti-receptor IL1RAP antibody 4B6 VL chain
atgagggcccctgctcagatttttggcatcttgttgctcttgtttccaggtaccagatgtgacatccagatgacccagtctccatcctccttatctgcctctctgggagaaagagtcagtctcacttgtcgggcaagtcaggacattggtagtagcttaaactggcttcagcaggaaccagatggaactattaaacgcctgatctacgccacatccagtttagattctggtgtccccaaaaggttcagtggcagtaggtctgggtcagattattctctcaccatcagcagccttgagtctgaagattttgtagactattactgtctacaatatgctagttctccgtggacgttcggtggaggcaccaagctggaaatcaaa

SEQ ID NO: 44 - protein, 127 aa, Artificial Sequence
  Synthetic anti-receptor IL1RAP antibody 4B6 VL chain
MRAPAQIFGILLLLFPGTRCDIQMTQSPSSLSASLGERVSLTCRASQDIGSSLNWLQQEPDGTIKRLIYATSSLDSGVPKRFSGSRSGSDYSLTISSLESEDFVDYYCLQYASSPWTFGGGTKLEIK

SEQ ID NO: 45 - DNA, 411 bp, Artificial Sequence
  Synthetic cDNA for anti-receptor IL1RAP antibody 4B6 VH chain
atgaactttgggttcagcttggttttccttgtccttgttttaaaaggtgtccagtgtgaagtgaagctggtggagtctgggggaggcttagtgaagcctggagggtccctgaaactctcctgtgcagcctctggattcactttccgtacctatgccatgtcttgggttcgccagactccagagaagaggctggagtgggtcgcatccattagtagtcgtggtagaatatactatccagacagtgtgaagggccgattcaccatctccagagataatgccaggaacatcctgtacctgcaaatgagcagtctgaggtctgaggacacggccatgtattactgtgcaagagacggtgattatggtagtagttacgactactggggccaaggcaccactctcacagtctcctca

SEQ ID NO: 46 - protein, 137 aa, Artificial Sequence
  Synthetic anti-receptor IL1RAP antibody 4B6 VH chain
MDFGFSLVFLVLVLKGVQCEVKLVESGGGLVKPGGSLKLSCAASGFTFRTYAMSWVRQTPEKRLEWVASISSRGRIYYPDSVKGRFTISRDNARNILYLQMSSLRSEDTAMYYCARDGDYGSSYDYWGQGTTLTVSS

SEQ ID NO: 47 - protein, 16 aa, Artificial Sequence
  synthetic peptide AC Pep-1
VKQKVPAPRYTVELAC